SEQUENCE LISTING (27 sequences)

SEQ ID NO: 1 - DNA, 2827 bp, Homo sapiens
ggcacgaggctccggcctctggactaggaaccgacagcccccctccccgcgtccctccctctctctccagccgttttggggaggggctctccacgctccggatagttcccgagggtcatccgcgccgcactcgcctttccgtttcgccttcacctggatataatttccgagcgaagctgcccccaggatgaccacgctggccggcgctgtgcccaggatgatgcggccgggcccggggcagaactacccgcgtagcgggttcccgctggaagtgtccactcccctcggccagggccgcgtcaaccagctcggcggcgtttttatcaacggcaggccgctgcccaaccacatccgccacaagatcgtggagatggcccaccacggcatccggccctgcgtcatctcgcgccagctgcgcgtgtcccacggctgcgtctccaagatcctgtgcaggtaccaggagactggctccatacgtcctggtgccatcggcggcagcaagcccaaggtgacaacgcctgacgtggagaagaaaattgaggaatacaaaagagagaacccgggcatgttcagctgggaaatccgagacaaattactcaaggacgcggtctgtgatcgaaacaccgtgccgtcagtgagttccatcagccgcatcctgagaagtaaattcgggaaaggtgaagaggaggaggccgacttggagaggaaggaggcagaggaaagcgagaagaaggccaaacacagcatcgacggcatcctgagcgagcgagcctcagcaccccaatcagatgaaggctctgatattgactctgaaccagatttaccactaaagaggaaacagcgcagaagccgaaccaccttcacagcagaacagctggaggaactggagcgtgcttttgagagaactcattaccctgacatttatactagggaggaactggcccagagggcgaagctcaccgaggcccgagtacaggtctggtttagcaaccgccgtgcaagatggaggaagcaagctggggccaatcaactgatggctttcaaccatctcattcccggggggttccctcccactgccatgccgaccttgccaacgtaccagctgtcggagacctcttaccagcccacatctattccacaagctgtgtcagatcccagcagcaccgttcacagacctcaaccgcttcctccaagcactgtacaccaaagcacgattccttccaacccagacagcagctctgcctactgcctccccagcaccaggcatggattttccagctatacagacagctttgtgcctccgtcggggccctccaaccccatgaaccccaccattggcaatggcctctcacctcagaattcaattcgtcataatctgtccctacacagcaagttcattcgtgtgcagaatgaaggaactggaaaaagttcttggtggatgctcaatccagagggtggcaagagcgggaaatctcctaggagaagagctgcatccatggacaacaacagtaaatttgctaagagccgaagccgagctgccaagaagaaagcatctctccagtctggccaggagggtgctggggacagccctggatcacagttttccaaatggcctgcaagccctggctctcacagcaatgatgactttgataactggagtacatttcgccctcgaactagctcaaatgctagtactattagtgggagactctcacccattatgaccgaacaggatgatcttggagaaggggatgtgcattctatggtgtacccgccatctgccgcaaagatggcctctactttacccagtctgtctgagataagcaatcccgaaaacatggaaaatcttttggataatctcaaccttctctcatcaccaacatcattaactgtttcgacccagtcctcacctggcaccatgatgcagcagacgccgtgctactcgtttgcgccaccaaacaccagtttgaattcacccagcccaaactaccaaaaatatacatatggccaatccagcatgagccctttgccccagatgcctatacaaacacttcaggacaataagtcgagttatggaggtatgagtcagtataactgtgcgcctggactcttgaaggagttgctgacttctgactctcctccccataatgacattatgacaccagttgatcctggggtagcccagcccaacagccgggttctgggccagaacgtcatgatgggccctaattcggtcatgtcaacctatggcagccaggcatctcataacaaaatgatgaatcccagctcccatacccaccctggacatgctcagcagacatctgcagttaacgggcgtcccctgccccacacggtaagcaccatgccccacacctcgggtatgaaccgcctgacccaagtgaagacacctgtacaagtgcctctgccccaccccatgcagatgagtgccctggggggctactcctccgtgagcagctgcaatggctatggcagaatgggccttctccaccaggagaagctcccaagtgacttggatggcatgttcattgagcgcttagactgtgacatggaatccatcattcggaatgacctcatggatggagatacattggattttaactttgacaatgtgttgcccaaccaaagcttcccacacagtgtcaagacaacgacacatagctgggtgtcaggctgagggttagtgagcaggttacacttaaaagtacttcagattgtctgacagcaggaactgagagaagcagtccaaagatgtctttcaccaactcccttttagttttcttggttaaaaaaaaaaaaaaaaaaaaaa

SEQ ID NO: 2 - DNA, 3517 bp, Homo sapiens
ccgtcagtgagttccatcagccgcatcctgagaagtaaattcgggaaaggtgaagaggaggaggccgacttggagaggaaggaggcagaggaaagcgagaagaaggccaaacacagcatcgacggcatcctgagcgagcgagcctcagcaccccaatcagatgaaggctctgatattgactctgaaccagatttaccactaaagaggaaacagcgcagaagccgaaccaccttcacagcagaacagctggaggaactggagcacgttgcttttgagagaactcattaccctgacatttatactagggaggaactggcccagagggcgaagctcaccgaggcccgagtacaggtctggtttagcaaccgccgtgcaagatggaggaagcaagctggggccaatcaactgatggctttcaaccatctcattcccgggggattccctcccactgccatgccgaccttgccaacgtaccagctgtcggagcactcttaccagcccacatctattccacaagctgtgtcagatcccagcagcaccgttcacagacctcaaccgcttcctccaagcactgtacaccaaagcacgattccttccaacccagacagcagctctgcctactgcctccccagcaccaggcatggattttccagctatacagacagctttgtgcctccgtcggggccctccaaccccatgaaccccaccattggcaatggcctctcacctcagaattcaattcgtcataatctgtccctacacagcaagttcattcgtgtgcagaatgaaggaactggaaaaagttcttggtggatgctcaatccagagggtggcaagagcgggaaatctcctaggagaagagctgcatccatggacaacaacagtaaatttgctaagagccgaagccgagctgccaagaagaaagcatctctccagtctggccaggagggtgctggggacagccctggatcacagttttccaaatggcctgcaagccctggctctcacagcaatgatgactttgataactggagtacatttcgccctcgaactagctcaaatgctagtactattagtgggagactctcacccattatgaccgaacaggatgatcttggagaaggggatgtgcattctatggtgtacccgccatctgccgcaaagatggcctctactttacccagtctgtctgagataagcaatcccgaaaacatggaaaatcttttggataatctcaaccttctctcatcaccaacatcattaactgtttcgacccagtcctcacctggcaccatgatgcagcagacgccgtgctactcgtttgcgccaccaaacaccagtttgaattcacccagcccaaactaccaaaaatatacatatggccaatccagcatgagccctttgccccagatgcctatacaaacacttcaggacaataagtcgagttatggaggtatgagtcagtataactgtgcgcctggactcttgaaggagttgctgacttctgactctcctccccataatgacattatgacaccagttgatcctggggtagcccagcccaacagccgggttctgggccagaacgtcatgatgggccctaattcggtcatgtcaacctatggcagccaggcatctcataacaaaatgatgaatcccagctcccatacccaccctggacatgctcagcagacatctgcagtcaacgggcgtcccctgccccacacggtaagcaccatgccccacacctcgggtatgaaccgcctgacccaagtgaagacacctgtacaagtgcctctgccccaccccatgcagatgagtgccctggggggctactcctccgtgagcagctgcaatggctatggcagaatgggccttctccaccaggagaagctcccaagtgacttggatggcatgttcattgagcgcttagactgtgacatggaatccatcattcggaatgacctcatggatggagatacattggattttaactttgacaatgtgttgcccaaccaaagcttcccacacagtgtcaagacaacgacacatagctgggtgtcaggctgagggttagtgagcaggttacacttaaaagtacttcagattgtctgacagcaggaactgagagaagcagtccaaagatgtctttcaccaactcccttttagttttcttggttaaaaaaaaaaacaaaaaaaaaaaccctccttttttcctttcgtcagacttggcagcaaagacatttttcctgtacaggatgtttgcccaatgtgtgcaggttatgtgctgctgtagataaggactgtgccattggaaatttcattacaatgaagtgccaaactcactacaccatataattgcagaaaagattttcagatcctggtgtgctttcaagttttgtatataagcagtagatacagattgtatttgtgtgtgtttttggtttttctaaatatccaattggtccaaggaaagtttatactctttttgtaatactgtgatgggcctcatgtcttgataagttaaacttttgtttgtactacctgttttctgcggaactgacggatcacaaagaactgaatctccattctgcatctccattgaacagccttggacctgttcacgttgccacagaattcacatgagaaccaagtagcctgttatcaatctgctaaattaatggacttgttaaacttttggaaaaaaaaagattaaatgccagctttgtacaggtcttttctatttttttttgtttattttgttatttgcaaatttgtacaaacatttaaatggttctaatttccagataaatgatttttgatgttattgttgggacttaagaacatttttggaatagatattgaactgtaataatgttttcttaaaactagagtctactttgttacatagtcagcttgtaaattttgtggaaccacaggtatttgggggcagcattcataattttcattttgtattctaactggattagtactaattttatacatgcttaactggtttgtacactttgggatgctacttagtgatgtttctgactaatcttaaatcattgtaattagtacttgcatattcaacgtttcaggccctggttgggcaggaaagtgatgtatagttatggacactttgcgtttcttatttaggataacttaatatgtttttatgtatgtattttaaagaaatttcactgcttctctgaactatgcgtactgcatagcatcaagtcttctctagagacctctgtagtcctgggaggcctcataatgtttgtagatacagaaagggagactgcatctaaagcaatggtcctttgtcaaacgagggattttgatccacttcaccattttgagttgagctttagcaaaagtttccctcataattctttgctcttgtttcagtccaggtggaggttggttttgtagttctgccttgaggaattatgtcaacactcatacttcatctcattctcccttctgccctgcagattagattacttagcac

SEQ ID NO: 3 - DNA, 3200 bp, Homo sapiens
ccgtttcgccttcacctggatataatttccgagcgaagtgcccccaggatgaccacgctggccggcgctgtgcccaggatgatgcggccgggcccggggcagaactacccgcgtagcgggttcccgctggaagtgtccactcccctcggccagggccgcgtcaaccagctcggcggcgtttttatcaacggcaggccgctgcccaaccacatccgccacaagatcgtggagatggcccaccacggcatccggccctgcgtcatctcgcgccagctgcgcgtgtcccacggctgcgtctccaagatcctgtgcaggtaccaggagactggctccatacgtcctggtgccatcggcggcagcaagcccaagcaggtgacaacgcctgacgtggagaagaaaattgaggaatacaaaagagagaacccgggcatgttcagctgggaaatccgagacaaattactcaaggacgcggtctgtgatcgaaacaccgtgccgtcagtgagttccatcagccgcatcctgagaagtaaattcgggaaaggtgaagaggaggaggccgacttggagaggaaggaggcagaggaaagcgagaagaaggccaaacacagcatcgacggcatcctgagcgagcgagcctcagcaccccaatcagatgaaggctctgatattgactctgaaccagatttaccactaaagaggaaacagcgcagaagccgaaccaccttcacagcagaacagctggaggaactggagcgtgcttttgagagaactcattaccctgacatttatactagggaggaactggcccagagggcgaagctcaccgaggcccgagtacaggtctggtttagcaaccgccgtgcaagatggaggaagcaagctggggccaatcaactgatggctttcaaccatctcattcccggggggttccctcccactgccatgccgaccttgccaacgtaccagctgtcggagacctcttaccagcccacatctattccacaagctgtgtcagatcccagcagcaccgttcacagacctcaaccgcttcctccaagcactgtacaccaaagcacgattccttccaacccagacagcagctctgcctactgcctccccagcaccaggcatggattttccagctatacagacagctttgtgcctccgtcggggccctccaaccccatgaaccccaccattggcaatggcctctcacctcagaattcaattcgtcataatctgtccctacacagcaagttcattcgtgtgcagaatgaaggaactggaaaaagttcttggtggatgctcaatccagagggtggcaagagcgggaaatctcctaggagaagagctgcatccatggacaacaacagtaaatttgctaagagccgaagccgagctgccaagaagaaagcatctctccagtctggccaggagggtgctggggacagccctggatcacagttttccaaatggcctgcaagccctggctctcacagcaatgatgactttgataactggagtacatttcgccctcgaactagctcaaatgctagtactattagtgggagactctcacccattatgaccgaacaggatgatcttggagaaggggatgtgcattctatggtgtacccgccatctgccgcaaagatggcctctactttacccagtctgtctgagataagcaatcccgaaaacatggaaaatcttttggataatctcaaccttctctcatcaccaacatcattaactgtttcgacccagtcctcacctggcaccatgatgcagcagacgccgtgctactcgtttgcgccaccaaacaccagtttgaattcacccagcccaaactaccaaaaatatacatatggccaatccagcatgagccctttgccccagatgcctatacaaacacttcaggacaataagtcgagttatggaggtatgagtcagtataactgtgcgcctggactcttgaaggagttgctgacttctgactctcctccccataatgacattatgacaccagttgatcctggggtagcccagcccaacagccgggttctgggccagaacgtcatgatgggccctaattcggtcatgtcaacctatggcagccaggcatctcataacaaaatgatgaatcccagctcccatacccaccctggacatgctcagcagacatctgcagttaacgggcgtcccctgccccacacggtaagcaccatgccccacacctcgggtatgaaccgcctgacccaagtgaagacacctgtacaagtgcctctgccccaccccatgcagatgagtgccctggggggctactcctccgtgagcagctgcaatggctatggcagaatgggccttctccaccaggagaagctcccaagtgacttggatggcatgttcattgagcgcttagactgtgacatggaatccatcattcggaatgacctcatggatggagatacattggattttaactttgacaatgtgttgcccaaccaaagcttcccacacagtgtcaagacaacgacacatagctgggtgtcaggctgagggttagtgagcaggttacacttaaaagtacttcagattgtctgacagcaggaactgagagaagcagtccaaagatgtctttcaccaactcccttttagttttcttggttaaaaaaaaaaaacaaaaaaaaaaaccctccttttttcctttcgtcagacttggcagcaaagacatttttcctgtacaggatgtttgcccaatgtgtgcaggttatgtgctgctgtagataaggactgtgccattggaaatttcattacaatgaagtgccaaactcactacaccatataattgcagaaaagattttcagatcctggtgtgctttcaagttttgtatataagcagtagatacagattgtatttgtgtgtgtttttggtttttctaaatatccaattggtccaaggaaagtttatactctttttgtaatactgtgatgggcctcatgtcttgataagttaaacttttgtttgtactacctgttttctgcggaactgacggatcacaaagaactgaatctccattctgcatctccattgaacagccttggacctgttcacgttgccacagaattcacatgagaaccaagtagcctgttatcaatctgctaaattaatggacttgttaaacttttggaaaaaaaag

SEQ ID NO: 4 - DNA, 470 bp, Homo sapiens
gaaggctttttctgatttgtgacaaaatgaatttatgcacactttcattgggtctttcggcaacttacacacattgaaaatgagctatggtacatattattatattctctttataaatgcatgtctgattgtacttgtaacaatattgtaatgaacggctgtgcagtaggcccagcgctgctgtgtctcgtcagaggaatagcttaccacgaacccctcagcatactgggaatctcttcctgaacaacgaatgtaaatttggtcaagtctactcttccgttcattcaattattttaagcatttgaattatttattgtatatcctaaatatatttctccttgggcagtgactagatttccactaatgtgtcttaatctatccctccagctggcacctgattttgtatccccctgtagcagcattactgaaatacataggcttatatacaatgcttctttcctgtatattct

SEQ ID NO: 5 - DNA, 466 bp, Homo sapiens
gaaggcttttctgatttttgacaaatgaattttgcacactttcatggtgtctttcggcaacttacacacattgaaaatgagctatgtacatatttttatattctctttataaatgcatgtctgattgtacttgtaacaatattgtaatgaacggctgtgcagtaggcccagcgctgctgtgtctcgtcagaggaatagcttaccacgaacccctcagcatactgggaatctcttcctgaacaacgaatgtaaatttggtcaagtctactcttccgttcattcaattattttaagcatttgaattatttattgtatatcctaaatatatttctcctttggcagtgactagatttccactaatgtgtcttaatctatccctccagctggcacctgattttgtatccccctgtagcagcattactgaaatacataggcttatatacaatgcttctttcctgtatattct

SEQ ID NO: 6 - DNA, 466 bp, Homo sapiens
gaaggctttttcgatttgtgacaaatgaattttgcacactttcatggtgtctttcggcaacttacacacattgaaaatgagctatggtacatattattatattctctttataaatgcatgtctgattgtacttgtaacaatattgtaatgaacggctgtgcagtaggcccagcgctgctgtgtctcgtcagaggaatagcttaccacgaacccctcagcatactgggaatctcttcctgaacaacgaatgtaaatttggtcaagtctactcttccgttcattcaattattttaagcatttgaattatttattgtatatcctaaatatatttctccttgggcagtgactagatttccactaatgtgtcttaatctatccctccagctggcacctgattttgtatccccctgtagcagcattactgaaatacataggcttatatacaatgcttctttcctgtatattc

SEQ ID NO: 7 - DNA, 466 bp, Homo sapiens
gaaggctttttctgatttttgacaaatgaattttgcacactttcatggtgtctttcggcaacttacacacatgaaaatgagctatgtacatatttttatattctctttataaatgcatgtctgattgtacttgtaacaatattgtaatgaacggctgtgcagtaggcccagcgctgctgtgtctcgtcagaggaatagcttaccacgaacccctcagcatactgggaatctcttcctgaacaacgaatgtaaatttggtcaagtctactcttccgttcattcaattattttaagcatttgaattatttattgtatatcctaaatatatttctcctttggcagtgactagatttccactaatgtgtcttaatctatccctccagctggcacctgattttgtatccccctgtagcagcattactgaaatacataggcttatatacaatgcttctttcctgtatattct

SEQ ID NO: 8 - DNA, 468 bp, Homo sapiens
gaaggctttttctgatttttgacaaatgaattttgcacactttcattggtgtctttcggcaacttacacacattgaaaatgagctatgtacatatttttatattctctttataaatgcatgtctgattgtacttgtaacaatattgtaatgaacggctgtgcagtaggcccagcgctgctgtgtctcgtcagaggaatagcttaccacgaacccctcagcatactgggaatctcttcctgaacaacgaatgtaaatttggtcaagtctactcttccgttcattcaattattttaagcatttgaattatttattgtatatcctaaatatatttctcctttggcagtgactagatttccactaatgtgtcttaatctatccctccagctggcacctgattttgtatccccctgtagcagcattactgaaatacataggcttatatacaatgcttctttcctgtatattct

SEQ ID NO: 9 - DNA, 469 bp, Homo sapiens
gaaggctttactgatttttggacaaatgaatttttgcacactttcattggtgtctttcggcaacttacacacattgaaaatgagctatgtacatatttttatattctctttataaatgcatgtctgattgtacttgtaacaatattgtaatgaacggctgtgcagtaggcccagcgctgctgtgtctcgtcagaggaatagcttaccacgaacccctcagcatactgggaatctcttcctgaacaacgaatgtaaatttggtcaagtctactcttccgttcattcaattattttaagcatttgaattatttattgtatatcctaaatatatttctcctttggcagtgactagatttccactaatgtgtcttaatctatccctccagctggcacctgattttgtatccccctgtagcagcattactgaaatacataggcttatatacaatgcttctttcctgtatattct

SEQ ID NO: 10 - DNA, 464 bp, Homo sapiens
tactgatttccggagcaaatgaattttgcacactattcattggtgtctttcggcaacttacacacattgaaaatgagctattgtacatatttttatattctcatttataaatgcatgtctgattgtacttgtaacaatattgtaatgaacggctgtgcagtaggcccagcgctgctgtgtctcgtcagaggaatagcttaccacgaacccctcagcatactgggaatctcttcctgaacaacgaatgtaaatttggtcaagtctactcttccgttcattcaattattttaagcatttgaattatttattgtatatcctaaatatatttctcctttggcagtgactagatttccactaatgtgtcttaatctatccctccagctggcacctgattttgtatccccctgtagcagcattactgaaatacataggcttatatacaatgcttctttcctgtatattct

SEQ ID NO: 11 - DNA, 483 bp, Homo sapiens
gaggtcactgagggatttttatttgcactgattttgctataaagtgtaactgaggtagagccaactgtccagggttggacaggggcaaggaacacaaggacccacggaaaggggagccagggtcctacgttgtggtgcagtgcctcccagtcatccgtataaacaataaattgggcaataaatagacggtggacaaggatcggagggacaggcagagaaggcttcaaggagtgtcaggctataggccttgaggaataaaacggtacagagttcgaactcactggggccatcttccaccctaaatttgttcagatgcagggtgagcacctgcagggttgtcatggtgctgttgaccctcacattggtcacggatccataggctggagtaaacacggaggtcttatgattgtagaagtggccgttgatagagaaccggtgtcgccggatgcgctgggcctcaccgggggcgcggcacttgggcct

SEQ ID NO: 12 - DNA, 833 bp, Homo sapiens
  misc_feature (550)..(550): Any nucleotide
gaggtcactgagggatttttatttgcactgattttgctataaagtgttactgaggtagagccaactgtccagggttggacaggggcaaggaacacaaggacccaaggaaaggggagccagggtcctacgttgtggtgcagtgcctcccagtcatccgtataaacaataaattgggcaataaatagacggtggacaaggatcggagggacaggcagagaaggcttcaaggagtgacaggctataggccttgaggaataaaacggtacagagttcgaactcactggggccatcttccaccctaaatttgttcagcagcagggtgagcacctgcagggttgtcatggtgctgttgaccctcacattggtcacggatccataggctggagtaaacacggaggtcttatgattgtagaagtggccgttgatagagaaccggtgtcgccggatgcgctgggcctcaccgggggcgcggcacttgggcctcctctggctcatgcaactggcgtcgctcttggtccgcatcagcttgggggcctcctctgcctcctcncagggccccgagctgtctgtggaactctcagccttgtgcactggctgaatgcttgggcccctggcttggatgatcccgttcntgggcgatggctccttttagaggcagcctaggcgtctgggcatcccatgaagtggagagaagtcacctggctccggtcatcctgcatctgcaagcggatgggccngctcagcccccaggcaatggtgaggagcccccttgatgaatagatcccttncttccctacgggtctaanctggaagctttgcccctaanggtacag

SEQ ID NO: 13 - DNA, 428 bp, Homo sapiens
tttttttttttttttttgaggtcactgagggatttttatttgcactgattttgctataaagtgttactgaggtagagccaactgtccagggttggacaggggcaaggaacacaaggacccaaggaaaggggagccagggtcctacgttgtggtgcagtgcctcccagtcatccgtataaacaataaattgggcaataaatagacggtggacaaggatcggagggacaggcagagaaggcttcaaggagtgacaggctataggccttgaggaataaaacggtacagagttcgaactcactggggccatcttccaccctaaatttgttcagcagcagggtgagcacctgcagggttgtcatggtgctgttgaccctcacattggtcacggatccataggctggagtaaacacggaggtcttatgattgta

SEQ ID NO: 14 - DNA, 614 bp, Homo sapiens
ggcattttcaagattttattgtaaaacagagctgaagtcacaggaagtagggaactttgcacccaacatatacagcattcattcgctgagtacctgccaagagcgaggctaggtgcttggattcttatgctaaataataacatctaactgatttggtgcttttggttcagttctgttctgagtgctttctatgtatgagctcatttattcctcagcaatcctaggaggtaggtattttcattatcaccttggttttacagatgagaggttaaggaacttgctcaaggtcacggctaagctaatgagtggccaacccaggttctgagactcaggcagtctggctgggagcccggagccccatccctctatgctgtaaggcttctgtattctagtgtcaacttccaagcttgccaaagtcccctgcaggcaacaagacgacctccccgtgggtctggactatgtgaccagctctctgagactcacccgataccgcagcgaataacctggtgtgcgggcggctcctcttcacgagctgtaatgtcaggctctcatcctggaggtgcccatcagacaccaggaggatgttccgtgaccctcgagcagggtacaataag

SEQ ID NO: 15 - DNA, 602 bp, Homo sapiens
  misc_feature (1)..(4): Any nucleotide
nnnnccttttttggcattttcaagattttattgtaaaacagagctgaagtcacaggaagtagggaactttgggnncnacatatacagcattcattcgctgagtacctgccaagagcgaggctaggtgcttggattcttatgctaaataataacatctaactgatttggtgcttttggttcagttctgttctgagtgctttctatgtatgagctcatttattcctcagcaatcctaggaggtaggtattttcattatcaccttggttttacagatgagaggttaaggaacttgctcaaggtcacggctaagctaatgagtggccaacccaggttctgagactcaggcagtctggctgggagcccggagccccatccctctatgctgtaaggcttctgtattctagtgtcaacttccaagcttgccaaagtcccctgcagggcaacaagacgacctccccgtgggtctggactatgtgaccagctctctgagactcacccgataccgcaggcgaataacctggtgtcgggcgggttctcttcacgagctggaatgtcaggctctcatccctgaggtgcccatcagacaccaggaggatggttcg

SEQ ID NO: 16 - DNA, 424 bp, Homo sapiens
ttttttttcatagaccaacattctttaatcacaaaggcacttgaggacccctacaaacccaaagtctctgccaagagtggccctgcagacgccccacctgccaccctccatccacccatccatccacacactcagagttcatcgggacctgcagagggctccacactaggcttgatgaaaatgcctggggccctcctcaaagttgctgacactttggggacgggaaggggtaaaagtagggctgctccttttggagctggagggaatagacctggagacagagttgaggcagtcgggctgtccaggttctaagcatcacagcttctgcactgggctctgaggagattctcagccagagcccagggaccactccagtagatgcagagaggggcctgcccaggggtcagggcagtgggtatcactg

SEQ ID NO: 17 - DNA, 535 bp, Homo sapiens
ttttttttttttttttttcatagaccaacattctttaatcacaaaggcacttgaggacccctacaaacccaaagtctctgccaagagtggccctgcagacgccccacctgccaccctccatccacccatccatccacacactcagagttcatcgtgacctgcagagggctccacactaggcttgatgaagatgcctggtgccctcctcaaagttgctgacactttggggacgggaaggggtagaagtagggctgctccttttggagctggagggaatagacctggagacagagttgaggcagtcgggctgtccaggttctaagcatcacagcttctgcactgggctctgaggagattctcagccagagcccagggaccactccagtagatgcagagaggggcctgccaggggtcagggcagtgggtatcactgggtgacatccagaatatcagagttcccatagtggtgcatgagtgggctcggtcctgcctcgtgccgaattcttgcctcgagggcaaatccccatatgattcg

SEQ ID NO: 18 - DNA, 608 bp, Homo sapiens
catttcaatagaactagctttatttacttatttatttatttaaacaaaagaaatggtttaaaagcaaatgcatatatgtaccaagggatggacatgacctggtacttacaaaggagctgctgtgtcataatggaaacagcatattaggagaaaaatagtatttcgtgtgctcctctggagccacgctcagtactaagcgccaggcaccgcattctgaggattctacctactaaacatactggactccagcctccgggcccttccctggctcagggtttccaatgccagcctcctcaatggcctccccaaacccagctagaattgcccagtctaggccaggggcagtggcccacacctgtaatcccagcactgtggggggccaaagtgggtggatcacttgaggcccagaagtcgagaacagcctggccaatatggggaaaccctggctttacaaaaaattatccaagcatgggggcacaaggatccccgtgccgaaatcttggccctcgagggcccaaatctcccaatagggaggcggataaaaatccgaaaaaatggcaaaaactgggttcctgggggaaaataggtattccgggtaaaaaatccca

SEQ ID NO: 19 - DNA, 243 bp, Homo sapiens
catttcaatagaactagctttatttacttatttatttatttaaacaaaagaaatggtttaaaagcaaatgcatatatgtaccaagggatggacatgacctggtacttacaaaggagctgctgtgtcataatggaaactgcatattaggagaaaaatagtatttcgtgtgctcctctggagccacgctcagaattaagcgcctggcaccgtattttgaggattctacctactaaacatactgga

SEQ ID NO: 20 - DNA, 441 bp, Homo sapiens
  misc_feature (357)..(357): Any nucleotide
tgcatttcaatagaactagctttatttacttatttatttatttaaacaaaagaaatggtttaaaagcaaatgcatatatgtaccaagggatggacatgacctggtacttacaaaggagctgctgtgtcataatggaaacagcatattaggagaaaaatagtatttcgtgtgctcctctggaccacgctcagtactaagcgccaggcaccgcattctgaggattctacctactaaacatactggactccagcctccgggcccttccctggctcaggtttccaatgccagcctcctcaatggcctccccaaacccagctagaattgcccagtctaggccaggggcagtggcccacaccntgtaatcccagcactgtgggngggccaaggtgggtggatcacttgaggccaggagttcgagaccagcctggccaatatggtgaa

SEQ ID NO: 21 - DNA, 499 bp, Homo sapiens
tgcatttcaatagaactagctttatttacttatttatttatttaaacaaaagaaatggtttaaaagcaaatgcatatatgtaccaagggatggacatgacctggtacttacaaaggagctgctgtgtcataatggaaacagcatattaggagaaaaatagtatttcgtgtgctcctctggaccacgctcagtactaagcgccaggcaccgcattctgaggattctacctactaaacatactggactccagcctccgggcccttccctggctcaggtttccaatgccagcctcctcaatggcctccccaaacccagctagaattgcccagtctaggccaggggcagtggcccacacctcgtaatcccagcactgtggggggccaaggtgggtggatcacttgaggccaggagttcgagaccagcctggccaatatggtgaaacctgtctttacaaaaattatccaggcatggtggcacatgtctcctcgtgccgaattct

SEQ ID NO: 22 - DNA, 499 bp, Homo sapiens
tgcatttcaatagaactagctttatttacttatttatttatttaaacaaaagaaatggtttaaaagcaaatgcatatatgtaccaagggatggacatgacctggtacttacaaaggagctgctgtgtcataatggaaacagcatattaggagaaaaatagtatttcgtgtgctcctctggactcacgctcagtactaagcgccaggcaccgcattctgaggattctacctactaaacatactggactccagcctccgggcccttccctggctcaggtttccaatgccagcctcctcaatggcctccccaaacccagctagaattgcccagtctaggccaggggcagtggccacacctgtaatcccagcactgtggggggccaaggtgggtggatcacttgaggccaggagttcgagaccagcctggccaattatggtgaaccctggtctttaaaaaaatttatcaggcatggtggaaatgtttcctcgtgccgaatcct

SEQ ID NO: 23 - DNA, 2087 bp, Homo sapiens
  misc_feature (775)..(775): Any nucleotide
ctgtgatctagccaggttgacttctacagcctcctcttgcattgccctgcacaatctccacattcacgtccggtagcctcactagtactcatcttccttcagttctcagttcagatgtcagttcttcaggaggccttctggaatgtctcaggccaggttaagtgtctcacttgagctcccacgaccctacaaccctctgagatttcgtaggtgtattacatcacatcttctgccttatttgactcttgttcgtttccctgtcggacagtcttagaaggtgggaccttgactattttattacattcccagtgcccagcttagtgcctggcacagggaactccttagaaaatatgtttgttgaatgaagtcagatctccatcacacccatgctgtcatgtgactacagagagccaccaacatcaagcacagacacaaagacactcatctcacacagggtgttagcccccagccctgtcccaggcccaatatactcacatccagtgaggtcactgagggatttttatttgcactgattttgctataaagtgttactgaggtagagccaactgtccagggttggacaggggcaaggaacacaaggacccaaggaaaggggagccagggtcctacgttgtggtgcagtgcctcccagtcatccgtataaacaataaattgggcaataaatagacggtggacaaggatcggagggacaggcagagaaggcttcaaggagtgacaggctataggccttgaggaataaaacggtacagaagtngcngaancntncacntggggnccantcntntnccannccnctaaantntntngtntncangncagncangggntgagcanccntgncaggngnttgntncatnggtngcntgttgacccntcacanttggtcacnggnatccatnaggctnggagtaaancacnggaggtctntantgattgtagaagtggcncgttgatnagagnaanccggtgtcngccggatgcgctgggcctncacncngggggcgcnggcacttgggcnctcctcntggnctcanntnnngcaactggcngtcgctcttggtccgcatcagnctggggggcctcnctctgcctncctcncaggggccccgangctgtcntgtggaactctcagccttgtgcactggctgaatgcttnggcccctgggctgtngatgttcccgttctggggcgatggctccntttagagggcagnctanggccgtctgggcatnccatgaggtgngnaggggaggtgcacctngctcccggtcatccntgcatctgcnagccggnatgggccgcctcnagncccccaggcaatgttgaggagnccncctcgatgatcagagtcccttnctntcctcacggtgtctcagctggaagctcnttgnccctcatggtagcagnttgtaggttttcnagcaggcctaagnagctccgacttctgaatggacttgctgtcactgatgggcacgtgagaactcggcagacagtcttccttcatcttctcttcctcttgtctgcagacctagaagttacttgcngtccagtcatcgcgcagtccagaagccactgcatgtgtcgtgtcctcgcttgcaccggagctgcgcccgcagcgcctgctgaccccaggcctcccgctgctcggggccaggcccgcccctcgccccgcccctcgcccctcgccccgccccgctccccgcctctcgcccggcacggccaaaggtgccgccccggtctctccgtcccgccccgagcacgcccctcactccgcaccggccaatggcaccgccccgtccgccccgctctgggaacgccggtaggttccgggtgcagggcggtggcctaggggcggcggggaagatttggggcctgcgacctgccaggtgtccccaggccattgttcagctcgccgcgcgctcccgagttcccgagggcgccccgggacgcgaggagaaagcagcccgactcagccccgagaggttcggtgtgcgggcagcggccagggactttgcggggtagcgcggggtggggcttggagtctggggaggcttatcagtaaagcc

SEQ ID NO: 24 - DNA, 3615 bp, Homo sapiens
  misc_feature (775)..(775): Any nucleotide
ctgtgatctagccaggttgacttctacagcctcctcttgcattgccctgcacaatctccacattcacgtccggtagcctcactagtactcatcttccttcagttctcagttcagatgtcagttcttcaggaggccttctggaatgtctcaggccaggttaagtgtctcacttgagctcccacgaccctacaaccctctgagatttcgtaggtgtattacatcacatcttctgccttatttgactcttgttcgtttccctgtcggacagtcttagaaggtgggaccttgactattttattacattcccagtgcccagcttagtgcctggcacagggaactccttagaaaatatgtttgttgaatgaagtcagatctccatcacacccatgctgtcatgtgactacagagagccaccaacatcaagcacagacacaaagacactcatctcacacagggtgttagcccccagccctgtcccaggcccaatatactcacatccagtgaggtcactgagggatttttatttgcactgattttgctataaagtgttactgaggtagagccaactgtccagggttggacaggggcaaggaacacaaggacccaaggaaaggggagccagggtcctacgttgtggtgcagtgcctcccagtcatccgtataaacaataaattgggcaataaatagacggtggacaaggatcggagggacaggcagagaaggcttcaaggagtgacaggctataggccttgaggaataaaacggtacagaagtngcngaancntncacntggggnccantcntntnccannccnctaaantntntngtntncangncagncangggntgagcanccntgncaggngnttgntncatnggtngcntgttgacccntcacanttggtcacnggnatccatnaggctnggagtaaancacnggaggtctntantgattgtagaagtggcncgttgatnagagnaanccggtgtcngccggatgcgctgggcctncacncngggggcgcnggcacttgggcnctcctcntggnctcanntnnngcaactggcngtcgctcttggtccgcatcagnctggggggcctcnctctgcctncctcncaggggccccgangctgtcntgtggaactctcagccttgtgcactggctgaatgcttnggcccctgggctgtngatgttcccgttctggggcgatggctccntttagagggcagnctanggccgtctgggcatnccatgaggtgngnaggggaggtgcacctngctcccggtcatccntgcatctgcnagccggnatgggccgcctcnagncccccaggcaatgttgaggagnccncctcgatgatcagagtcccttnctntcctcacggtgtctcagctggaagctcnttgnccctcatggtagcagnttgtaggttttcnagcaggcctaagnagctccgacctaaaaagggaaagacgcacatgtactcctagggtgtggcagggcctcagcttctggcctttggcttccctggcctctgtttcctcatttgtaacaggagtgtgacagcacccttctcgccggccaggcataaccagaggagaccagactgccatcngcaatgcacccggatggttctgcacatggccctggtggggggcatgggggtcgggctgaggaggaggcgagactcacgggacaggcctgcgtctggtccaggatgtccagggtgccgacactttcactatcacaggtaaggccctggcagcctccctgccctccancctgcgttgcttncctcacagtaaatgccctggacttgcttttggcattgtacagcctcttgcttttccccatatcacttcctacaaggcctgctgtggctgctgcngggccaccctgaggggtgcctgcttcttccccaccctgactttccaaaggcgcnagtggaaggtgatggccctgtgagtaanggccctgaagtctgcagtaagccactttccctcaacgctgtcntcccttcccgaagggaaagccacagccctggccagcagacgtgggatgtgagccacactggtaatttcaatcttctagtacatgttaggaggtgatatggtttggatgtgtgtccccacccaaatctcatgtggaattgtaattcccagtgttggaggtggggccaggtgggaggtgttntggatcatgggagcnagatccctcatgagtggcttgggccatnccccgtggtgnataagtgagcttttgctctgagttcacacgaggtctggtcatttaaaagtgntgtggcaccaccctccactccctctctcttgctcctgcntttcgccatgtgaagtgcctgctnccccgttcgccttccaacatgactggaggctccttgaggcctnccctggaagccgagcagatgccagcaccacacttcttgtaaagcctccagaaccgtgagccaactaaatctctntttctttgtaaattacccagcttcaggtatttctttatagcaacacaagaatggcctaatacagaggggaaaatgaaacaggtgaaattagcattaataacacattttattgaactcaatatatctaaattactatttgtcaacatgcaatcaatgtaaaagttataatgaggccctgtacacacctatttcgtataaagtgcctgccatctggagcctcttttctattgcccatgcctcccagccatggtcatgtgtagcttggggatcccctattggacaggctcaaaaagaagcaccccatcactgtcccccagggcaccccatcagatctctgaacaagtgctcagaacccctccacctaaaggctgcccaggatgagggggatggcaaatgtgaagactcccccgcccaacctgcacacctaagagctcctacttctgaatggacttgctgtcactgatgggcacgtgagaactcggcagacagtcttccttcatcttctcttcctcttgtctgcagacctagaagttacttgcngtccagtcatcgcgcagtccagaagccactgcatgtgtcgtgtcctcgcttgcaccggagctgcgcccgcagcgcctgctgaccccaggcctcccgctgctcggggccaggcccgcccctcgccccgcccctcgcccctcgccccgccccgctccccgcctctcgcccggcacggccaaaggtgccgccccggtctctccgtcccgccccgagcacgcccctcactccgcaccggccaatggcaccgccccgtccgccccgctctgggaacgccggtaggttccgggtgcagggcggtggcctaggggcggcggggaagatttggggcctgcgacctgccaggtgtccccaggccattgttcagctcgccgcgcgctcccgagttcccgagggcgccccgggacgcgaggagaaagcagcccgactcagccccgagaggttcggtgtgcgggcagcggccagggactttgcggggtagcgcggactcctggacttccaacaagggccgctgcgcaggcgctgcaatcactgcaggcacttgtctgcatgcctggtggggcttggagtctggggaggcttatcagtaaagcc

SEQ ID NO: 25 - DNA, 2109 bp, Homo sapiens
  misc_feature (775)..(775): Any nucleotide
ctgtgatctagccaggttgacttctacagcctcctcttgcattgccctgcacaatctccacattcacgtccggtagcctcactagtactcatcttccttcagttctcagttcagatgtcagttcttcaggaggccttctggaatgtctcaggccaggttaagtgtctcacttgagctcccacgaccctacaaccctctgagatttcgtaggtgtattacatcacatcttctgccttatttgactcttgttcgtttccctgtcggacagtcttagaaggtgggaccttgactattttattacattcccagtgcccagcttagtgcctggcacagggaactccttagaaaatatgtttgttgaatgaagtcagatctccatcacacccatgctgtcatgtgactacagagagccaccaacatcaagcacagacacaaagacactcatctcacacagggtgttagcccccagccctgtcccaggcccaatatactcacatccagtgaggtcactgagggatttttatttgcactgattttgctataaagtgttactgaggtagagccaactgtccagggttggacaggggcaaggaacacaaggacccaaggaaaggggagccagggtcctacgttgtggtgcagtgcctcccagtcatccgtataaacaataaattgggcaataaatagacggtggacaaggatcggagggacaggcagagaaggcttcaaggagtgacaggctataggccttgaggaataaaacggtacagaagtngcngaancntncacntggggnccantcntntnccannccnctaaantntntngtntncangncagncangggntgagcanccntgncaggngnttgntncatnggtngcntgttgacccntcacanttggtcacnggnatccatnaggctnggagtaaancacnggaggtctntantgattgtagaagtggcncgttgatnagagnaanccggtgtcngccggatgcgctgggcctncacncngggggcgcnggcacttgggcnctcctcntggnctcanntnnngcaactggcngtcgctcttggtccgcatcagnctggggggcctcnctctgcctncctcncaggggccccgangctgtcntgtggaactctcagccttgtgcactggctgaatgcttnggcccctgggctgtngatgttcccgttctggggcgatggctccntttctgaaaacagacaactgcagggctgagatggctgccggcagagaaagggagctcaggccaaggagaagagaccagcagcccagagggcagnctanggccgtctgggcatnccatgaggtgngnaggggaggtgcacctngctcccggtcatccntgcatctgcnagccggnatgggccgcctcnagncccccaggcaatgttgaggagnccncctcgatgatcagagtcccttnctntcctgcacacctaagagctcctacttctgaatggacttgctgtcactgatgggcacgtgagaactcggcagacagtcttccttcatcttctcttcctcttgtctgcagacctagaagttacttgcngtccagtcatcgcgcagtccagaagccactgcatgtgtcgtgtcctcgcttgcaccggagctgcgcccgcagcgcctgctgaccccaggcctcccgctgctcggggccaggcccgcccctcgccccgcccctcgcccctcgccccgccccgctccccgcctctcgcccggcacggccaaaggtgccgccccggtctctccgtcccgccccgagcacgcccctcactccgcaccggccaatggcaccgccccgtccgccccgctctgggaacgccggtaggttccgggtgcagggcggtggcctaggggcggcggggaagatttggggcctgcgacctgccaggtgtccccaggccattgttcagctcgccgcgcgctcccgagttcccgagggcgccccgggacgcgaggagaaagcagcccgactcagccccgagaggttcggtgtgcgggcagcggccagcaggcacttgtctgcatgcctggtggggcttggagtctggggaggcttatcagtaaagcc

SEQ ID NO: 26 - DNA, 2123 bp, Homo sapiens
  misc_feature (775)..(775): Any nucleotide
ctgtgatctagccaggttgacttctacagcctcctcttgcattgccctgcacaatctccacattcacgtccggtagcctcactagtactcatcttccttcagttctcagttcagatgtcagttcttcaggaggccttctggaatgtctcaggccaggttaagtgtctcacttgagctcccacgaccctacaaccctctgagatttcgtaggtgtattacatcacatcttctgccttatttgactcttgttcgtttccctgtcggacagtcttagaaggtgggaccttgactattttattacattcccagtgcccagcttagtgcctggcacagggaactccttagaaaatatgtttgttgaatgaagtcagatctccatcacacccatgctgtcatgtgactacagagagccaccaacatcaagcacagacacaaagacactcatctcacacagggtgttagcccccagccctgtcccaggcccaatatactcacatccagtgaggtcactgagggatttttatttgcactgattttgctataaagtgttactgaggtagagccaactgtccagggttggacaggggcaaggaacacaaggacccaaggaaaggggagccagggtcctacgttgtggtgcagtgcctcccagtcatccgtataaacaataaattgggcaataaatagacggtggacaaggatcggagggacaggcagagaaggcttcaaggagtgacaggctataggccttgaggaataaaacggtacagaagtngcngaancntncacntggggnccantcntntnccannccnctaaantntntngtntncangncagncangggntgagcanccntgncaggngnttgntncatnggtngcntgttgacccntcacanttggtcacnggnatccatnaggctnggagtaaancacnggaggtctntantgattgtagaagtggcncgttgatnagagnaanccggtgtcngccggatgcgctgggcctncacncngggggcgcnggcacttgggcnctcctcntggnctcanntnnngcaactggcngtcgctcttggtccgcatcagnctggggggcctcnctctgcctncctcncaggggccccgangctgtcntgtggaactctcagccttgtgcactggctgaatgcttnggcccctgggctgtngatgttcccgttctggggcgatggctccntttagagggcagnctanggccgtctgggcatnccatgaggtgngnaggggaggtgcacctngctcccggtcatccntgcatctgcnagccggnatgggccgcctcnagncccccaggcaatgttgaggagnccncctcgatgatcagagtcccttnctntctggtgctctacaagcacagatgtgtcactgagcttcctcacggtgtctcagctggaagctcnttgnccctcatggtagcagnttgtaggttttcnagcaggcctaagnagctccgacttctgaatggacttgctgtcactgatgggcacgtgagaactcggcagacagtcttccttcatcttctcttcctcttgtctgcagacctagaagttacttgcngtccagtcatcgcgcagtccagaagccactgcatgtgtcgtgtcctcgcttgcaccggagctgcgcccgcagcgcctgctgaccccaggcctcccgctgctcggggccaggcccgcccctcgccccgcccctcgcccctcgccccgccccgctccccgcctctcgcccggcacggccaaaggtgccgccccggtctctccgtcccgccccgagcacgcccctcactccgcaccggccaatggcaccgccccgtccgccccgctctgggaacgccggtaggttccgggtgcagggcggtggcctaggggcggcggggaagatttggggcctgcgacctgccaggtgtccccaggccattgttcagctcgccgcgcgctcccgagttcccgagggcgccccgggacgcgaggagaaagcagcccgactcagccccgagaggttcggtgtgcgctgcaatcactgcaggcacttgtctgcatgcctggtggggcttggagtctggggaggcttatcagtaaagcc

SEQ ID NO: 27 - DNA, 4794 bp, Homo sapiens
  misc_feature (453)..(453): Any nucleotide
ttttttgagaccaggagaaataactttatttgaataggacccaagacagcatattgggctaaggcccagaggtaaggttccaaaaccgcagtcaaagctcatcaaccaaatggactctacttcccagcaaccttgcagttagtgcaaccaacaaaaggcctgctggggaatgtatttgccactaaattccccaagtatgccaacattacaaaaaagataggtttttcatcataattgaatttccacaaacctccccaatcacaagtattataagtggaagtaaaaaatcacattttacagatctcaaacttgtcttcaacatttagttcatcatcttcaaaaaatagctcccctgcctaattcattagctatatgatctaaccaagcagcaacaagatggccaggccatggcaatcctcttcccatttccctcgtgccgaattcggagaggtntatttttttattttttttggcattttcaagattttnattngntaaaacagagctgaagtcacaggaagtangggaactttgcacccaacnatatacnangcattcattncgnctgagtaccntgccaangagcgaggctnaggtgcttggattcttatgctaaataataacatctaactgatttggtgcttttggttcagttctgttctgagtgctttctatgtatgagctcatttattcctcagcaatcctaggaggtaggtattttcattatcaccttggttttacagatgagaggttaaggaacttgctcaaggtcacggctaagctaatgagtggccaacccaggttctgagactcaggcagtctggctgggagcccggagccccatccctctatgctgtaaggcttctgtattctagtgtcaacttccaagcttgccaaagtcccctgcagggcaacaagacgacctccccgtgggtctggactatgtgaccagctctctgagactcacccgataccgcaggcgaataacctggtgtgcgggcggctcctcttcacgagctgtaatgtcaggctctcatcctggaggtgcccatcagacaccaggaggatgttccgtgaccctcgagcagggtacaataagctaagatatcggagtgttttccagaagtctgtgttccccatggtaggtgtggcagacatgatgaactctgctgccgcggtattgcttgtgatatgcttaggatacgaaaatagctccttgtaacctgtgccgaactggataatatttactttctgcttctcacccaccaaggacagcgcatgcaaggcgatttccttggcttgcaagaatgtcacaccctccatggaactggagcagtcaagacaaataatcacttcgctctcattggctaggtcagggaggtcgacatcgagatcgggttgaaagacaagcatgcaagcctcgctttctttttctggatgtttttcaacccacattcttgggagataggcagcagacaaaccgatgtggagagaaaatccactgctgtctaaggagctgccttccatggtgctaatgacagctttgcagtctgtgcgcttttgtttcagttcatgagtatcactgaaaatgaattcaatcacgtacggcatctcaatagacatagtcaaagagaagctttgctttgttcctatttcttttatacaaatcttctctactgtatcctgaaggttttcattcaaagccttgtcctgttgccagggtgctacggtggcgggcatgaaaaagacaccaacagtgcccaggatgctgagttctgtgatgtaggtaatttttataagaaccttagccttagggggtaagtttccaacacttacagtaaaaacgtccggagcatcctgactcatcaggtaagcgccatggccctgggtcacggcttctaggtactcttgctgggcttcttccttctctttaatctctccaactatgtgcttcccattgatgaaggcttcgaagccacacacagcggccttgtcatccaaaggaaagatatattttgcctcaatgggcacgtgacttttatttgtgtgaaccaagttcccagaagcatcctggaggccggccttggtgctgctggaagttttggcatctggtaactggtaatcttcaacctttgaaaaatttgaaaactcaggtctgtattcctctaattcagtatgatcactaggatgaaagtcctttatctgatctccaggcatggaaaatttaataatatatttcattttaacctgattggttttatagacaacaaattcatcatcctcaaagtctgtggtgacagaggctgtttgtgaaactccatgcacactgtcgtagcctggtggtgcttcagttaaggaaaagtccttctcatgtaagtccatacactttccgagggctacgtcacaaatgagcaggagtctggtgccatctgtctctcccgggtgtgagtacttgatacttgtactgagcgaatcactgaaataaatcccacttccaaggtttccgacgtctgttctttgcacaccacgatcttccactactttgggtaaaagcaaccctcgacacaagattcccacgatgttttgtacaggagaaccatgcaacaagggcctcacattaccaagtttgctcaaaaactctgtggtttcattcactctgccaactctaaatatctgcaagacatccactgggctcttactgtgatgattctgcaaaacctcttttctaaccctgagaaattcttcagtattctgttcaacatgctcaattttgcacctcaaagctcggtatttggccagggatggtgggttgggtttggacaaattagtttcacagacattaaccatgtctcttattagctggcagaggtctgctttcttagccaatagtcccaggttcacttctttgggcattgtgcctttgtgaggtatcagtctgtaaaactctgtcatcatcttttgcaattgctctgctgtttctccatttttcagtgctgcctttactagaaggagaatcccctctgccttgctcacatcgttgaggctaatcctgttcactggcttgagaagcatgtgttccaggtggcccagggcctctgcccaaatcatctctactaaatcgctcacctcttggctcagagtgcttgaattcatgacttcctccaaaagcaatgcttgcaattgttcagatgctaattgggttgcttcaggtgtgaaatgttctcttagtagaaatccttgtttcttcagttcttcaatgtaattttcaaagtattcacttgcatcttcagaggttttctttatagcaaactgtcttctagtctccatgccatcatccaggaggaagtgtgaggatatcaggaaaggacagtccctggagtcccgcgaacactgaagctccaccaccacagcttcctggcctccctccattcccactttctccaaggtgttatattttgcaacttcaaaatcttgaggaagatgaggaatttcaacattctgcataccaaactcagtgagttccacagtgtcttcctcctctgtggcactgtccgggcatagaccttctgttttcacttcagaactgctcgccttctgatcaggaggtggtgtgatgtccaggggcttataaggatcataattctttacatccaagagtctcttttctctgatagatttccatataaaatctgggtttgcaatatgaacgtggttcttttggatagaattcagttggtactgactcagaacatcagcattatctaagattatatgtgtgcactgaggatttaacgaaaaggaaaactttccgccattttccttaatgtcagtttgtagctttttcttctgctgctgaggtaagtacttcactttcaaacagaagatacaatttgcaaagattcccatcaccatcctgcctagaggatccagctagctccggacgctccccgattccgcgctccttgctcgcggtaattccgtaatttaggctttccctgcccccggggctgggcgggcgccactgcgccggaaccccgccccgcaccaggcgggcgcagcgtcccaagcagggacgatagccggaacgcgcccggaagttcagggctttcagtttcatatttcaggagggattttgtcaatgaggattaatacaaagctcggctttgagagaggtgggtggatggtgctgtcctttaactgagaaatggaagggattcacaaggccgggaaagaagaaaatttcgggaaagttcgtggaaacaggagagtgagataataaatttactttaagccttcctgcgtttgaaattcgtgacagtgtaactagttggaaatatatttatctgccattcaagagaggcagggggctggagatacagatctgagattcactgaaatgggaataaagcaatggggaagattcagatttttaggatactgaataaaatgagaatagaaaggatagagtagagaatacagtggaaagggtatggggttgaatcctgactccacaatttattaccaagggggctggggacaaggtaatttaccaagtttcatcattacaacctactctatagggttgtctaagaaacaacaagagattctgggggaggagagagactgattcctagagttaccacatcgtaatgctcaaaatgtccaatcttcaacaaaaaattatgaggcatgcaaaaaaaaaggcgaaaaaatggccaattcaaaggaaaaaagaaatgaacagaaattgtccctgagctgaggaagcacagacataggaattattagacaaat

(19) United States
(12) Patent Application Publication
(10) Pub. No.: US 2004/0110227 A1
(43) Pub. Date: Jun. 10, 2004

(54) Methods and systems for identifying putative fusion transcripts, polypeptides encoded therefrom and polynucleotide sequences related thereto and methods and kits utilizing same

(76) Inventors: Erez Levanon, Petach Tikva (IL); Amir Toporik, Mishmar Hashiva (IL); Pini Akiva, Yavne (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(21) Appl. No.: 10/391,530

(22) Filed: Mar. 19, 2003

Related U.S. Application Data
(60) Provisional application No. 60/365,076, filed on Mar. 19, 2002.

(51) Int. Cl. G01N 33/53; G06F 19/00; G01N 33/48; G01N 33/50

(52) U.S. Cl. 435/7.1; 702/19

(57) ABSTRACT

The present invention provides a method of identifying putative fusion transcripts. The method comprises: (a) computationally aligning a first database of annotated polynucleotide sequences with a second database of expressed polynucleotide sequences; and (b) identifying in the second database an expressed polynucleotide sequence complementary to at least two non-contiguous sequences of the first database, the at least two non-contiguous sequences being selected from the group consisting of non-homologous polynucleotide sequences mapped to different chromosomes, polynucleotide sequences mapped to different loci of a single chromosome and polynucleotide sequences mapped to a single locus and not being a part of a splice isoform, the expressed polynucleotide sequence identified being a putative fusion transcript.

This Application claims the benefit of priority from U.S. Provisional Patent Application No. filed 60/365,076 Mar. 19, 2002.

### BACKGROUND AND FIELD OF THE INVENTION

The present invention relates to the field of chromosomal/RNA transcript rearrangements (also referred to herein as genetic rearrangements). More particularly, the present invention relates to methods of identifying chimeric transcripts generated by abnormal rearrangements of nucleic acid sequences such as chromosomes or RNA transcripts, databases storing nucleic acid sequences encoding identified fusion transcripts, oligonucleotides derived therefrom and methods and kits utilizing same. Additionally, the present invention relates to polynucleotide sequences involved in the chimeric events and oligonucleotides derived therefrom which can be used as important tools for the diagnosis and treatment of numerous disorders involving genetic rearrangements, such as cancer.

Cancer is a genetic disorder in which a series of mutations subvert the normal developmental program of a cell and allow it to proliferate without constraint. Accumulation of deleterious mutations appears to represent the basis of cancer progression [Kinzler K W and Vogelstein B (1997) Nature 386:7C)1-763]. While these mutations can take many forms, the most characteristic form of genetic change in cancer cells is karyotypic instability with aneuploidy and chromosomal rearrangements, particularly, balanced translocations. By joining together previously unlinked chromosomal arms, balanced translocations can result in the creation of hybrid genes with altered expression patterns for potential oncogenes or tumor suppressor genes.

The presence or absence of specific translocations has therapeutic and prognostic implications [Sanchez-Garcia 1 (1997) Annu. Rev. Genet. 31:429-453]. These translocations serve as markers of the malignant state and can be either the cause or the consequence of the transformed state. For example, the Philadelphia chromosome is a specific t(9:22)(q34;q11) translocation that fuses the Breakpoint Cluster Region gene (BCR) and the ABL oncogenes [De Klein et al. (1982) Nature 300:765-767]. This fusion is thought to represent the crucial event in the development of chronic myelogenous leukemia (CML), though it can also appear later in the course of various forms of leukemia. Though, non-random chromosomal translocations were initially thought to occur exclusively in tumors of the hematopoietic system, solid tumors, like sarcomas, also display characteristic translocations, suggesting that the development of an unstable chromosomal state increases the likelihood of translocations which in turn increase the likelihood of tumor progression [Rabbits T H (1994) Nature 372:143-149; Sanchez-Garcia I (1997) Annu. Rev. Genet. 31:429-453].

Pathological conditions, other than cancer, are also known to be associated with chromosomal rearrangements. These include for example: multiple sclerosis associated with a chromosomal translocation, mental impairment, delayed membranous ossification of the cranium, blepharophimosis, ptosis, epecanthus inversus syndrome and hyperprolinemia.

Though some translocation events appear to occur sporadically (i.e., non-randomally), most translocations are believed to arise from recombination, whereby double-stranded DNAs are broken and rejoined in ways that alter the linkage relationship of the genes near the breakpoints. At least three different recombination pathways, which can cause translocations have been proposed.

One such pathway is homologous recombination, where identical sequences on nonhomologous chromosomes cross-over, resulting in new linkages of nonhomologous chromosome arms. This model is supported by the fact that repetitive sequences in the human genome such as ALU elements or retrotransposons such as LINE elements are occasionally observed at translocation breakpoints [Kato et al. (1991) Gene 97:239-244]. Studies from a number of model organisms, particularly yeast, indicate that the presence of a double strand break greatly stimulates the process of homologous recombination, via strand invasion of a linear single-stranded end into complementary double-stranded sequences elsewhere in the genome [Peters (1991) Recombination in yeast. “The molecular and cellular biology of yeast Saccharomyces”. Broach J R et al. Cold Spring Harbor Laboratory Press, Cold Spring Harbor, N.Y.]. However, it appears that homologous recombination is a minor pathway for translocation-formation in human cancers.

Another proposed pathway is site-specific recombination, which requires a site-specific recombinase, as well as specific DNA recognition sequences for recombination. In humans, B and T cell precursors go through a site-specific recombinatorial process known as the V(D)J rejoining that is essential for their maturation. V(D)J rejoining assembles antigen receptor variable genes by making double-strand breaks at specific recombination signal sequences and then rejoining non-contiguous intrachromosomal segments. Many reciprocal translocations associated with lymphoid malignancies involve a V(D)J cleavage-rejoining site at the breakpoint [Rabbits T H (1994) Nature 372:143-149]. This suggests that such translocations result from aberrant nonhomologous rejoining events which can occur during the normal site-specific process. The V(D)J cleavage site represents one breakpoint; potential sources of the second breakpoint necessary for translocation include both physiological (e.g., transcription, replication, repair) and environmental (e.g., x-rays, free radicals). It has been recently demonstrated that the V(D)J recombinase can function as a transposase, capable of actively inserting cleaved DNA ends into random targets [Hiom et al. (1998) 94:463-470].

Yet another proposed pathway for recombination-mediated translocation is non-homologous recombination. This is an inherent imprecise form of recombination which appears to be a major pathway for double strand break repair in human cells [Meuth M (1989) Illegitimate recombination in mammalian cells. “Mobile DNA” Berg D E and Howe M M American Society for Microbiology, Washington D.C.]. In this form of recombination, no specific sequences are present at the breakage sites. The recombination joints normally involve at most, a few (i.e., <5) overlapping bases. Based on analysis of the breakpoints, it is believed that most cancer causing chromosomal translocations occur by non-homologous end-joining of simultaneous double strand breaks that are present on separate non-homologous chromosomes. This type of recombination has been studied in mammalian systems by analyzing sites of integration and excision of DNA viruses and transfected linear DNA markers, as well as by determining the genetic components required for normal recombination repair of V(D)J site-specific cleavage events [Roth et al. (1995) Current Biol. 5:496-499]. Overall, it seems that there may be multiple end-joining pathways, which utilize amongst other proteins, Rad50, Mre11, Xrs2, Ku70, Ku80, the DNA-dependent protein kinase, DNA ligase 4 and XRCC4p [Hendrickson F A (1997) Am. J. Hum. Genet. 61:795-800].

The underlying causes of the translocations found in human tumors are believed to result from physiological, genetic, and/or environmental conditions which increase double-stranded DNA breaks or decrease their repair thereby increasing the likelyhood of translocations in cells. For example, lymphoid malignancies have associated translocations involving V(D)J recombination sites (Finger et al. Science 1986 234:982-985) The fact that V(D)J rejoining is a normal developmental process limited to lymphoid precursor cells underscores the point that the presence of a double-stranded DNA break at a specific chromosomal site makes that sire a hotspot for translocations. As another example, several rare recessive human disorders have been identified, including Bloom's Syndrome, ataxia telangiectasia, and Nijmegen Breakage Syndrome, whose hallmarks are chromosomal instability, hypersensitivity to DNA damaging agents, and early onset of a variety of malignancies. Analysis of the genes that are defective in these syndromes suggests that their normal functions are to minimize natural double-stranded DNA breaks or to halt progression of the cell cycle until breaks ate repaired [Brown et al. Proc. Natl. Acad. Sci. USA 1997 94:1840-1845; Carney et al. Cell 1998 93:477-486; Chaganti et al. Proc. Natl. Acad. Sci. USA 1974 71:4508-12; Ellis et al. Cell 1995 83:655-666, Epstein et al Medicine 1966 45-177-221; Kasten et al. Cell 1992 71:587-597; Krepinsky et al. Human Genetics 1979 50:151-6; Varon et al. Cell 1998 93:467-476; Watt et al. Genetics 1996 144:935-945; and Yamagata et al. Proc. Natl. Acad. Sci., USA 1998 95:8733-8738]. Disrupted biological activity of these genes leads to higher levels of double-stranded DNA breaks, which can be anomalously rejoined.

Realizing the fundamental role chromosomal translocations play in human physiology and in the development of malignancies, resulted in the development of numerous assays for unveiling translocation-associated genes and identification of new prognostic indicators.

Accordingly, a number of cytological techniques based upon chemical staining have been developed, which produce longitudinal patterns (i.e., bands) on condensed chromosomes. The banding pattern of each chromosome within an organism permits unambiguous identification of each chromosome type and accrurate detection of some important chromosomal abnormalities such as translocations and inversions [Latt (1976) Annu, Rev. of Biophys, and Bioeng. 5:1-37]. However such methods require cell culturing and preparation of high quality metaphase spreads, which is time consuming and labor intensive and freaquently unfeasible. For example, cells from many tumor types are difficult to culture and often do not represent the original tumor population. Furthermore, conventional banding analysis does not enable the detection of structural aberrations involving less than 3-15S megabases.

Hybridization-based assays [e.g., fluorescence in-situ hybridization (FISH), southern blot analysis, spectral karyotyping (SKY) and RT-PCR] which recognize translocation products based on genetic content have solved some of the shortcomings associated vital the traditional cytological methods. These methods make use of labeled fragments of single-stranded or double stranded DNA or RNA which are hybridized to complementary sites on chromosomal DNA. Hybridization with target specific probes is used to map the locations of particular genes in the genome [Harper and Saunders (1981) Proc. Natl. Acad. Sci. 78:44584460]. For example, fluorescent probes that hybridize to whole chromosomes, to specific portions of chromosomes or to specific genes (FISH), can be used to identify certain parts of chromosomes such as centromeres and to quantify chromosomes within a tumor cell.

One of the most frequent problems associated with hybridization-based methods is probe preparation. Specific chromosomes of interest are separated from other chromosomes using flow-sorting of synchronized populations of dividing cells, a technically rigorous procedure requiring highly specialized and expensive equipment (e.g., a fluorescence-activated cell sorter). Such sorting leads to the recovery of only a small quantity of material, and as such, the isolated DNA needs to be extracted and cloned into cosmid libraries. To generate the probe, the cosmid library must be expanded, DNA extracted again and nick-translated in the presence of labeled nucleotide prior to hybridization to human metaphase spreads and detection of the specific chromosomes [Pinkel, D. et al. (1986) Proc. Natl. Acad. Sci. USA 83:2934-2938].

One essential requirement for specific chromosome painting is the prehybridization of the probe with total human DNA in-order to prevent human repeat sequences, which are not chromosome specific, from participating in the in-situ hybridization reaction. Other serious limitations of this approach include cost-effectiveness and limited ability of flow-sorted libraries. Finally, probes made from flow sorted chromosome libraries do not enable identification of the regions of the respective chromosomes brought together by rearrangements since longitudinal differentiation (banding) of the specific chromosome is lost. Therefore, the approach is not effective in identifying either breakpoint sites in rearrangements or deletions or other events in which only a single chromosome is affected.

Isolation of human-chromosome specific DNA can also be effected using the polymerase chain reaction (PCR) to specifically amplify the human chromosome or chromosomal region of interest. However a recent attempt to prepare probes from such material resulted in spotted chromosomes with a high background signal making it impossible to observe any longitudinal differentiation of the target sequence.

Identification of cells having chromosomal rearrangements at known breakpoints on the affected chromosomes has been accomplished using as probes, sets of cosmids that flank the breakpoint.

Cosmid probes flanking a chromosomal breakpoint region have also been used to detect known chromosomal rearrangements. For example, a cosmid probe specific to the p-arm of chromosome 16 has enabled visualization of the rearrangement associated with acute non-lymphocytic leukemia [Dauwerse, J. et al. (1990) Cytognet. Cell Genet. 53:126-128]. Although detection was effected, the intensity of the signal was relatively weak, raising doubt that it could be reliably used to identify the breakpoint in the majority of cells. Such a method can be used effectively only in cases where the breakpoint flanking regions have been precisely identified and isolated. In the vast majority of cases the sites are unknown and there is no effective method of identification.

There is thus a widely recognized need for, and it would be highly advantageous to have, methods of systematically identifying novel chromosomal rearrangements, RNA transcript chimerism and methods and kits utilizing information derived therefrom for diagnosing, and/or treating a variety of disorders associated with genetic rearrangements.

### SUMMARY OF THE INVENTION

According to one aspect of the present invention there is provided a method of identifying putative fusion transcripts, the method comprising: (a) computationally aligning a first database of annotated polynucleotide sequences with a second database of expressed polynucleotide sequences; and (b) identifying in the second database an expressed polynucleotide sequence complementary to at least two non-contiguous sequences of the first database, the at least two non-contiguous sequences being selected from the group consisting of non-homologous polynucleotide sequences mapped to different chromosomes, polynucleotide sequences mapped to different loci of a single chromosome and polynucleotide sequences mapped to a single locus and not being a pan of a splice isoform, the expressed polynucleotide sequence identified being a putative fusion transcript.

According to another aspect of the present invention there is provided a method of identifying transition points in fusion transcripts, the method comprising: (a) computationally aligning a first database of annotated polynucleotide sequences with a second database of expressed polynucleotide sequences; and (b) selecting in the second database an expressed polynucleotide sequence complementary to at least two non-contiguous sequences of the first database, the at least two non-contiguous sequences being selected from the group consisting of non-homologous polynucleotide sequences mapped to different chromosomes, polynucleotide sequences mapped to different loci of a single chromosome and polynucleotide sequences mapped to a single locus and not being a part of a splice isoform, and (c) identifying within the putative fusion transcript at least one nucleic acid sequence region exhibiting transition point between a first contiguous sequence of the at least two non-contiguous sequences and a second contiguous sequence of the at least two non-contiguous sequences, thereby identifying the transition points in the fusion transcripts.

According to yet another aspect of the present invention there is provided a method of identifying polynucleotide sequences associated with a disorder associated with genetic rearrangements, the method comprising: (a) computationally aligning a first database of annotated polynucleotide sequences with a second database of expressed polynucleotide sequences derived from tissues characterized by disorders associated with genetic rearrangements; (b) identifying in the second database an expressed polynucleotide sequence complementary to at least two non-contiguous sequences of the first database, the at least two non-contiguous sequences being selected from the group consisting of non-homologous polynucleotide sequences mapped to different chromosomes, polynucleotide sequences mapped to different loci of a single chromosome and polynucleotide sequences mapped to a single locus and not being a part of a splice isoform, and (c) identifying the non-contiguous polynucleotide sequences of the first database to thereby identify the polynucleotide sequences associated with disorders associated with genetic rearrangements.

According to further features in preferred embodiments of the invention described below, the method further comprising the step of testing the polynucleotide sequences being associated with the disorder associated with genetic rearrangements for pathogenic potential under physiological conditions following step (c).

According to still another aspect of the present invention there is provided a method of identifying polypeptides resulting from putative fusion events, the method comprising: (a) computationally aligning a first database of annotated polynucleotide sequences with a second database of expressed polynucleotide sequences; (b) identifying in the second database expressed polynucleotide sequences complementary to at least two non-contiguous sequences of the first database, the at least two non-contiguous sequences being selected from the group consisting of non-homologous polynucleotide sequences mapped to different chromosomes, polynucleotide sequences mapped to different loci of a single chromosome and polynucleotide sequences mapped to a single locus and not being a part of a splice isoform, the expressed polynucleotide sequences identified being fusion transcripts, and (c) selecting from the fusion transcripts at least one fusion transcript including an open reading frame spanning at least one of the at least two non-contiguous sequences, thereby identifying the polypeptides resulting from the putative fission event.

According to an additional aspect of the present invention there is provided a kit useful for detecting genetic rearrangements, the kit comprising at least one oligonucleotide being designed and configured to be specifically hybridizable with at least one fusion transcript of the fusion transcripts set forth in the file “translocated_transcripts126.txt.gz”.

According to still further features in the described preferred embodiments wherein the at least one oligonucleotide is labeled.

According to still further features in the described preferred embodiments wherein the at least one oligonucleotide is attached to a solid substrate.

According to still further features in the described preferred embodiments wherein the solid substrate is configured as a microarray and whereas the at least one oligonucleotide includes a plurality of oligonuceleotides each being capable of hybridizing with a specific fusion transcript of the fusion transcript set forth in the file “translocated_transcripts126.txt.gz” and each being attached to the microarray in a regio-specific manner.

According to still further features in the described preferred embodiments wherein the at least one oligonucleotide is designed and configured for DNA staining.

According to still further features in the described preferred embodiments wherein the at least one oligonucleotide is designed and configured for RNA staining.

According to yet an additional aspect or the present invention there is provided a computer readable storage medium comprising data stored in a retrievable manner, the data including sequence information of at least a portion of the fusion transcripts set forth in file “translocated/transcripts126.txt.gz”.

According to still further features in the described preferred embodiments wherein the data further includes additional information specific to each transcript of the at least a portion of the fusion transcripts.

According to still further features in the described preferred embodiments wherein the additional information includes any of the following or any combination thereof: (i) genes functionally or structurally related to each transcript of the at least a portion of the fusion transcripts, (ii) a sequence length of each transcript of the at least a portion of the fusion transcripts; (iii) open reading frames and/or regulatory sequences associated with each transcript of the at least a portion of the fusion transcripts; (iv) transition point sequence between each transcript of the at least a portion of the fusion transcripts (v) pathological abundance; (vi) chromosomal snapping of each transcript of the at least a portion of the fusion transcripts; (vii) causative genetic event selected from the group consisting of a deletion and translocation, an insertion, erroneous splicing and a trans-splicing; (viii) EST-jump value; (ix) hotspot sequences; and (x) fusion event abundance.

According to still further features in the described preferred embodiments wherein the additional information is set forth in the file “chimeric_contigs_information”.

According to still further features in the described preferred embodiments wherein the database further includes information pertaining to generation of the data and potential uses of the data.

According to still an additional aspect of the present invention there is provided a system for generating a database of fusion transcripts, the system comprising a processing unit, the processing unit executing a software application configured for: (a) computationally aligning a first database of annotated polynucleotide sequences with a second database of expressed polynucleotide sequences; (b) identifying in the second database all expressed polynucleotide sequence complementary to at least two non-contiguous sequences of the first database, the at least two non-contiguous sequences being selected from the group consisting of non-homologous polynucleotide sequences mapped to different chromosomes, polynucleotide sequences mapped to different loci of a single chromosome and polynucleotide sequences mapped to a single locus and not being a part of a splice isoform; and (c) storing the fusion transcripts as retrievable data.

According to still further features in the described preferred embodiments wherein the software application is further configured for annotating the fusion transcripts stored and whereas the annotation is effected according to data derived from sequences or other databases.

According to a further aspect of the present invention there is provided a system for generating a database of nucleic acid sequences of transition points in fusion transcripts, the system comprising a processing unit, the processing unit executing a software application, configured for: (a) computationally aligning a first database of annotated polynucleotide sequences with a second database of expressed polynucleotide sequences; (b) selecting in the second database an expressed polynucleotide sequence complementary to at least two non-contiguous sequences of the first database, the at least two non-contiguous sequences being selected from the group consisting of non-homologous polynucleotide sequences mapped to different chromosomes, polynucleotide sequences mapped to different loci of a single chromosome and polynucleotide sequences mapped to a single locus and not being a part of a splice isoform, (c) identifying within the putative fission transcript at least one nucleic acid sequence region exhibiting transition point between a first contiguous sequence of the at least two non-contiguous sequences and a second contiguous sequence of the at least two non-contiguous sequences; and (d) storing the nucleic acid sequences of transition points in fusion transcripts as retrievable data.

According to still further features in the described preferred embodiments wherein the software application is further configured for annotating the nucleic acid sequences of transition points in fusion transcripts stored and whereas the annotation is effected according to data derived from sequences or other databases.

According to yet a further aspect of the present invention there is provided a system for generating a database of polypeptide encoding nucleic acid sequences resulting from putative fusion events, the system comprising a processing unit, the processing unit executing a software application configured for: (a) computationally aligning a first database of annotated polynucleotide sequences with a second database of expressed polynucleotide sequences; (b) identifying in the second database expressed polynucleotide sequences complementary to at least two non-contiguous sequences of the first database, the at least two non-contiguous sequences being selected from the group consisting of non-homologous polynucleotide sequences mapped to different chromosomes, polynucleotide sequences mapped to different loci of a single chromosomes and polynucleotide sequences mapped to a single locus and not being a part of a splice isoform, the expressed polynucleotide sequences identified being fusion transcripts; and (e) identifying from the fusion transcripts the polypeptide encoding nucleic acid sequences including an open reading frame spanning at least one of the at least two non-contiguous sequences; (d) storing the polypeptide encoding nucleic acid sequences resulting from the putative fusion as retrievable data.

According to still further features in the described preferred embodiments wherein the software application is further configured for annotating the polypeptide encoding nucleic acid sequences and whereas the annotation is effected according to data derived from sequences or other databases.

According to still a further aspect of the present invention there is provided a method of detecting a nucleic acid sequence chimerism indicative of predisposition for disorders associated with genetic rearrangements in a subject, the method comprising: (a) identifying a fusion transcript indicative of the nucleic acid sequence chimerism, (b) generating at least one oligonucleotide being complementary to the fusion transcript; (c) contacting a biological sample obtained from the subject with the at least one oligonucleotide; and (d) detecting a level of binding between the at least one oligonucleotide and the fusion transcript to thereby detect the nucleic acid sequence chimerism indicative of the predisposition for disorders associated with genetic rearrangements in the subject.

According to still further features in the described preferred embodiments wherein the at least one oligonucleotide being complementary to the fusion transcript is complementary to a transition point within the fusion transcript.

According to still further features in the described preferred embodiments wherein the step of identifying the fusion transcript indicative of the nucleic acid sequence chimerism is effected by: (i) computationally aligning a first database of annotated polynucleotide sequences with a second database of expressed polynucleotide sequences; and (ii) identifying in the second database expressed polynucleotide sequences complementary to at least two non-contiguous sequences of the first database, the at least two noncontiguous sequences being selected from the group consisting of non-homologous polynucleotide sequences mapped to different chromosomes, polynucleotide sequences mapped to different loci of a single chromosome and polynucleotide sequences mapped to a single locus and not being a part of a splice isoform, the expressed polynucleotide sequences identified being fusion transcripts.

According to still further features in the described preferred embodiments the method further comprising the step of testing the putative fusion transcript for the presence of at least one sequence element selected from the group consisting of a sequence repeat, a pseudogene sequence, a restriction site, a transposable element, a homologous sequence, a sequence direction, overhang length, a splice consensus site, an intron length, a transcript length, alignment score, a hotspot sequence, a vector sequence, a gap, a sequence conservation and an EST jump.

According to still further features in the described preferred embodiments wherein the first annotated database includes sequences of a type selected from the group consisting of genomic sequences, expressed sequence tags, contigs, complementary DNA (cDNA) sequences, pie-messenger RNA (mRNA) sequences and mRNA sequences.

According to still farther features in the described preferred embodiments wherein the second database includes sequences of a type selected from the group consisting of expressed sequence tags, contigs, complementary DNA (cDNA) sequences, pre-messenger RNA (mRNA) sequences and mRNA sequences.

According to still further features in the described preferred embodiments wherein the putative fusion transcripts are selected from the group consisting of translocation products, deletion products, duplication products, paracentric inversions, pericentric inversions, transpositions, ring chromosomes, trans-splicing products and trans-transcription products.

According to still further features in the described preferred embodiments wherein the at least one oligonucleotide is attached to a solid substrate.

According to still further features in the described preferred embodiments wherein the solid substrate is configured as a microarray and whereas the at least one oligonucleotide includes a plurality of oligonucleotides each attached to the microarray in a regio-specific manner.

According to still further features in the described preferred embodiments wherein the at least one oligonucleotide is labeled and whereas step (d) is effected by quantifying the label.

According to a supplementary aspect of the present invention there is provided a method of identifying putative mutagenic agents, the method comprising: (a) exposing a cell to a plurality of mutagens; (b) identifying a mutagen of the plurality of mutagens which induces expression of a fusion transcript in the cell as a result of exposure of the cell thereto, thereby identifying the putative mutagenic agent.

According to still further features in the described preferred embodiments wherein the identifying is effected on RNA target molecules of the plurality of cells.

The present invention successfully addresses the shortcomings of the presently known configurations by providing methods for systematically identifying novel chromosomal rearrangements, RNA transcript chimerism and methods and kits utilizing information derived therefrom for diagnosing, and/or treating a variety of disorders associated with genetic rearrangements

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a graph showing degree type 3 window size analysis of chromosome 6. The width of the window is 2 mega base pairs (Mb). The coordinates on the chromosome mark the start point of the sampling window,

FIGS. 2a-c are schematic illustrations showing different modes of EST jump. FIG. 2a illustrates an EST jump=0., wherein the alignments of the chimeric sequence to chromosomes A and B are adjacent to each otherl FIG. 2b illustrates an EST jump>0, wherein the alignments of the chimeric sequence to chromosomes A and B are distant from each other. The positive value of the EST jump refers to the number of intervening base pairs located between the two alignments; FIG. 2c illustrates an EST jump<0, wherein the alignments of the chimeric sequence to chromosomes A and B overlap. The negative value of the EST jump refers to the number of base pairs which overlap the two alignments.

FIG. 3 is a histogram depicitng the distribution of the positive EST jump lengths. The positive value is indicative of insertion of unaligned base pairs between the two alignments as described in FIG. 2b.

FIG. 4 is a histogram depicting the distribution of the negative EST jump lengths. The negative EST jump value is indicative of a repetitive segment joint to both alignments as described in FIG. 2c.

FIG. 5 is a graph depicting the distribution of chimeric sequences per cDNA libraries.

FIG. 6 is a graph depicting the distribution of the percent of chimeric sequences per cDNA libraries.

FIG. 7 is a histogram depicting the distribution of total chimeric sequences per chimeric event.

FIG. 8 is a histogram depicting the distribution of translocation chimeric sequences per chimeric event.

FIG. 9 is a histogram depicting the distribution of deletion chimeric sequences per chimeric event.

FIG. 10 is a histogram depicting the number of cDNA libraries supporting any chimeric event.

FIG. 11 is a histogram depicting the number of cDNA libraries supporting a chimeric event resulting from translocation.

FIG. 12 is a histogram depicting the number of cDNA libraries supporting chimeric event resulting from deletion.

FIG. 13 is a histogram depicting the distribution of chimeric events supporting any fusion between 2 genes.

FIG. 14 is a histogram depicting the distribution of chimeric events supporting a fusion between 2 genes which result from translocation.

FIG. 15 is a a histogram depicting the distribution of chimeric events supporting a fusion between 2 genes which result from deletion.

FIG. 16 is a graph depicting total number of degree type II chimeric events involving a, certain contig and different partner contigs.

FIG. 17 is a graph depicting degree type II chimeric events which result from translocation and involve a certain contig and different partner contigs.

FIG. 18 is a graph depicting degree type II chimeric events which result from deletion and involve a certain contig and different partner contigs.

FIG. 19 are the results of alignment or The chimeric sequences supporting the chimeric events of contig Z40694 to the human genome using the BLAT algorithm (UCSC). Alignmemts are presented for each chimeric event. The following format is used: <coordinate of start of alignment on chimeric sequence> <coordinate of end of alignment on chimeric sequence> <chimeric sequence length> <identity score> <chromnsome> <strand> <coordinate of start of alignment on DNA sequence> <coordinate of end of alignment on DNA sequence>. Due to a competition between the alignments to the genome the alignments which where chosen are marked in bold. The serial numbers correspond to the serial row number of Table 13.

FIG. 20 is the results of alignment of the chimeric sequences supporting the chimeric events or contig R31638 to the human genome using the BLAT algorithm (UCSC). Alignments are presented for each chimeric event. The following format is used: <coordinate of start of alignment on chimeric sequence> <coordinate of end of alignment on chimeric sequence> <chimeric sequence length> <identity score> <chromiosome/<strand> <Coordinate of start of alignment on DNA sequence> <coordinate of end of alignment on DNA sequence. Due to a competition between the alignments to the genome the alignments which where chosen are marked in bold. The serial numbers correspond to the serial row number of Table 14.

FIG. 21 is the results of alignment of the chimeric sequences supporting the chimeric events of contig HSOΛ3MR to the human genome using the BLAT algorithm (UCSC). Alignments are presented for each chimeric event. The following format is used coordinate of start of alignment on chimeric sequence> <coordinate of end of alignment on chimeric sequence> <chimeric sequence length> <identity score> <chromosomne> <strand> <coordinate of start of alignment on DNA sequence> <coordinate of end of alignment on DNA sequence>. Due to a competition between the alignments to the genome the alignments which where chosen are marked in bold. The serial numbers correspond to the serial row number of Table 15.

FIG. 22 is the results of alignment of the chimeric sequences supporting the chimeric events of contig D12334 to the human genome using the BLAT algorithm (UCSC). Alignments are presented for each chimeric event. The following format is used: <coordinate of start of alignment on chimeric sequence> <coordinate of end of alignment on chimeric sequence> <chimeric sequence length> <identity score><chromosome> <strand> <coordinate of start of alignment on DNA sequence> <coordinate of end of alignment on DNA sequence>. Due to a competition between the alignments to the genome the alignments which where chosen are marked in bold The serial numbers correspond to the serial row number of Table 16.

FIG. 23 is a description genomic breakpoints in the D12334 contig. The indicated coordinates designate the aligned coordinates of the chimeric ESTs to the genome. The following format is used: <DNA1> <coordinates1> <DNA2> <coordinates2> <chimeric event i.d.>.

FIG. 24 is the results of alignment of the chimeric sequences supporting the chimeric events of contig Z38201 to the hum an genome using the BLAT algorithm (UCSC). Alignments are presented for each chimeric event. The following format is used: <coordinate of start of alignment on chimeric sequence> <coordinate of end of alignment on chimeric sequence> <chimeric sequence length> <identity score><chromosome> <strand> <coordinate of start of alignment on DNA sequence> <coordinate of end of alignment on DNA sequence>. Due to a competition between the alignments to the genome the alignments which where chosen are marked in bold. The serial numbers correspond to the serial row number of Table 17.

FIG. 25 is the results of alignment of the chimeric sequences supporting the chimeric events of contig T05149 to the human genome using the BLAT algorithm (UCSC). Alignments are presented for each chimeric event. The following format is used: <coordinate of start of alignment on chimeric sequence> <coordinate of end of alignment on chimeric sequence> <chimeric sequence length> <identity score> <chromosome> <strand> <coordinate of start of alignment on. DNA sequence> <coordinate of end of alignment on DNA sequence>. Due to a competition between the alignments to the genome the alignments which where chosen are marked in bold. The serial numbers correspond to the serial row number of Table 18.

FIG. 26 is a description of genomic breakpoints in the R27953 contig. The indicated coordinates designate the aligned coordinates of the chimeric ESTs to the genome. The following format is used: <DNA1> <coordinates1> <DNA2> <coordinates2> <chimeric event i.d.>.

FIG. 27 is the results of alignment of the chimeric sequences supporting the chimeric events of contig R27953 to the human genome using the BLAT algorithm (UCSC). Alignments are presented for each chimeric event. The following format is used. <coordinate of start of alignment on chimeric sequence> <coordinate of end of alignment on chimeric sequence>/chimeric sequence length> <identity score> <chromosome> <strand> <coordinate of start of alignment on DNA sequence> <coordinate of end of alignment on DNA sequence>. Due to a competition between the alignments to the Genome the alignments which where chosen are marked in hold. The serial numbers correspond to the serial row number or Table 19.

FIG. 28 is a histogram depicting a degree type III analysis. Note that each number on the X axis represents a gene or a contig (10+11, and 13+14+15 night represent one gene). The analysis Spans over a region of 1 mega base pairs on chromosome 3. The Y-axis represents the number of chimeric events occurring in gene.

FIG. 29 is the results of alignment of the chimeric sequences supporting the chimeric events of contig T10051 to the human genome using the BLAT algorithm (UCSC) Alignments are presented for each chimeric event. The following format is used: <coordinate of start of alignment on chimeric sequence> <coordinate of end of alignment on chimeric sequence> <chimeric sequence length> <identity score> <chromosome> <strand> <coordinate of start of alignment on DNA sequence> <coordinate of end of alignment on DNA sequence>. Due to a competition between the alignments to the genome the alignments which where chosen are marked in bold. The third alignment depicts the alignment results from the NCBI BLAST against the human genome. The following format is used: <e-score> <start on EST> <end on EST> <% identity> <chromosome> <strand> <coordinate of start of alignment on DNA sequence> <coordinate of end of alignment on DNA sequence>. The serial numbers correspond to the serial row number of Table 20.

FIG. 30 is a sequence repeat distribution in the RBM5 gene. The following format is used: <start coordinate of repeat in the contig> <end coordinate of repeat in the contig> <type of alignment: complement (c), +,−> <name of repeat> <type of repeat>. A=4, B=2, C=3, D=7 and E=1,5 and 6 numbers represent the chimeric event shown in Table 20.

FIG. 31 is a description of genomic breakpoints in the HSKERELP contig. The indicated coordinates designate the aligned coordinates of the chimeric ESTs to the genome. <DNA1> <coordinates1> <DNA2> <coordinates2> <chimeric event i.d.>

FIG. 32 illustrates the results of the alignment of the chimeric sequences supporting the chimeric events of the HSKERELP contig to the human genome using the BLAT algorithm (UCSC). Alignments are presented for each chimeric event. The following format is used: <coordinate of start on alignment on chimeric sequence> <coordinate of end of alignment on chimeric sequence> <chimeric sequence length><identity score> <chrojnosome> <strand> <coordinate of start of alignment on DNA sequence> <coordinate of end or alignment on DNA sequence>. Due to a competition between the alignments to the genome the alignments which where chosen are marked in bold. The serial numbers correspond to the serial row number of Table 21.

FIG. 33 is a flowchart outlining an approach used by the present invention in order to uncover genetic rearrangements. A—cleaning of expressed sequence data from abundant gene regions, vector contaminants, low complexity gene regions and sequence repeats; B—all against all BLAST analysis resulting in binaric expressed sequences and genomic data and farther data filtration; C—generation of expressed sequence coordinates, using genomic data; D—use of SuperSep application software for scoring alignments; E—identification of expressed sequences aligning to at least two non-contiguous genomic regions; F—clustering of fused expressed sequences into defined fusion events; G—filtering-out of artificial fusion events and annotation.

FIG. 34 illustrates a system designed and configured for generating a database of chimeric transcript sequences generated according to the teachings of the present invention.

FIG. 35 is am illustration of a remote configuration of the system described in FIG. 34.

FIGS. 36a-b are examples of the format of the files provided in the CD-ROM enclosed herewith. (“translocated_transcripts126.txt.gz”, “chimeric_contig_information.txt”).

FIG. 37 is a schematic illustration of the transcription product of the t(2; 13)(q35;q 14) translocation. The 97 KDa PAX-3;FKHR chimeric transcript comprises the 5′ UTR, N-terminal and DNA-binding domain of PAX-3 (red) and the C-terminal transactivation domain of FKHR (blue).

FIG. 38 is a schematic illustration of PAX-3;FKHR EST sequence alignment along genomic DNA. Alignment identifies chromosomal region hits; chromosome 2 encompassing PAX-3 and chromosome 13 including FKHR. The putative breakpoint is mapped to the border-line between the two chromosomal regions. Alignment score is provided by the indicated color code.

FIG. 39 is a schematic illustration of Calmodulin-I;MGP chimeric EST sequence alignment along genomic DNA. Alignment identifies chromosomal region hits; chromosome 14 encompassing Calmodulin-I and chromosome 12 including MGP. The putative breakpoint is mapped to the border-line between the two chromosomal regions. Alignment score is provided by the indicated color code.

FIG. 40 is a schematic illustration of the transcription product of the t(14;12)(q24-31;p13.1-123) translocation. The Calmodulin-1:MGP chimeric transcript comprises the complete coding sequence and regulatory regions of calmodulin-I (red) and the C-terminal transactivation domain of MGP (blue).

FIG. 41 is a schematic illustration of a chimeric EST (FE65L2;AD037) along genomic DNA. Alignment identifies chromosomal region hits; chromosome 5 including FE65L2 and chromosome 10 including AD037. The putative breakpoint is mapped to the border-line between the two chromosomal regions. Alignment score is provided by the indicated color code.

FIG. 42 is a schematic illustration of the putative fusion transcript FE65L2;AD037. The chimeric transcript comprises a truncated coding sequence of AD037 (red), a unique sequence consequential of the fusion event (yellow) and the C-terminal regulatory domain of FE65L2 (blue).

FIG. 43 is a schematic illustration of a chimeric EST (IL1RN;ADPRTL1) along genomic DNA. Alignment identifies chromosomal region hits, chromosome 2 including IL1RN and chromosome 13 including ADPRTL 1. The putative breakpoint is mapped to the border-line between the two chromosomal regions. Alignment score is provided by the indicated color code.

FIG. 44 is a schematic illustration of the putative fusion transcript IL1RN;ADPRTL1. The chimeric transcript comprises a truncated coding sequence of ADPRTL1 (red), a unique sequence consequential of the fusion event (yellow) and the C-terminal regulatory domain of IL1 RN (blue).

FIG. 45 is a schematic illustration of a chimeric EST (THOX2;SDF2L1) along genomic DNA. Alignment identifies chromosomal region hits; chromosome 15 including THOX2 and chromosome 22 including SDF2L1. The putative breakpoint is mapped to the border line between the two chromosomal regions.

FIG. 46 is a schematic illustration of the putative fusion transcript THOX2;SDF2L1. The chimeric transcript comprises a 5′UTR (white) and truncated coding sequence of THOX2 (green), a unique sequence consequential of the fusion event (red) and the C-terminal coding and regulatory domain of SDF2L1 (yellow). A stop codon UGA is indicated.

FIG. 47 is a schematic illustration of a chimeric EST (SARM;LOC120398) along genomic DNA. Alignment identifies chromosomal region hits at chromosome 11 and chromosome 17.

FIGS. 48a-b are schematic illustrations of alternative translocation models for the SARM;LOC120398 gene fusion.

FIG. 49 illustrates the format of the file “degree_{—}2_analysis.txt” provided in the CD-ROM enclosed herewith.

### DESCRIPTION OF THE PREFERRED EMBODIMENTS

The present invention is of methods of identifying putative fusion transcripts (e.g., chimeric transcripts), transition points included therein, proteins encoded therefrom and polynucleotide sequences related thereto, which, can be used in kits and methods for determining fusion transcript expression. Specifically, the fusion transcript molecules and related oligonucleotides of the present invention can be used to determine a subject's predisposition to disorders associated with genetic rearrangements, such as cancer, and to identify putative mutagenic agents.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings described in the Examples section. The invention is capable of other embodiments or of being practiced or carried out in various ways. Also, it is to be understood that the phraseology and terminology employed herein is for the purpose of description and should not be regarded as limiting.

Terminology

As used herein, the tern “oligonucleotide” refers to a single stranded or double stranded oligomer or polymer of ribonucleic acid (RNA) or deoxyribonucleic acid (DNA) or mimetics thereof. This term includes oligonucleotides composed of naturally-occurring bases, sugars and covalent internucleoside linkages (e.g., backbone) as well as oligonucleotides having non-naturally-occurring portions which function similarly. Such modified or substituted oligonucleotides are often preferred over native forms because of desirable properties such ass for example, enhanced cellular uptake, enhanced affinity for nucleic acid target and increased stability ill the presence of nucleases.

The phrase “fusion transcripts” (also termed “chimerism”) refers to transcripts, which result from chromosomal translocations or deletions or from abnormal RNA transcript processing. Fusion transcripts may result from chromosomal rearrangements including translocations (described in details in the Background section) or from transcript splicing errors such as in trans-splicing of two independently transcribed and heterologous pre-mRNAs, the result of such trans-splicing is referred to herein as “chimeric transcripts”. The mechanism of trans-splicing, which is nearly identical to that of conventional cis-splicing, proceeds via two phosphoryl transfer reactions. Trans-splicing may also refer to a different process, where an intron of one pre-mRNA interacts with an intron of a second pre-mRNA, enhancing the recombination of splice sites between two conventional pre-mRNAs. This type of trans-splicing was postulated to account for transcripts encoding a human immunoglobulin variable region sequence linked to the endogenous constant region in a transgenic mouse [Shimizu et al. (1989) Proc. Natl. Acad. Sci. USA 86:8020].

The phrase “transition point” refers to a unique nucleic acid sequence which is generated by the chromosomal/transcript rearrangements described above and as such is indicative thereof.

The phrase “single nucleotide polymorphism” or SNP, refers to a variation from the most frequently occurring base at a particular nucleic acid position.

The phrase “open reading frame” (ORF) refers to a nucleotide sequence, which could potentially be translated into a polypeptide. Such a stretch of sequence is uninterrupted by a stop codon. An ORF that represents the coding sequence for a full protein begins with an ATG “start” codon and terminates with one of the three “stop” codons. For the purposes of this application, an ORF may be any part of a coding sequence, with or without start and/or stop codons. For an ORF to be considered as a good candidate for coding for a bona fide cellular protein, a minimum size requirement is often set, for example., a stretch of DNA or RNA that would code for a protein of 50 amino acids or more. An ORF is not usually considered an equivalent to a gene or locus until a phenotype is associated with a mutation in the ORF, an mRNA transcript for a gene product generated from the ORF's DNA has been detected, and/or the ORF's protein product has been identified.

The term “complementary” refers to nucleic acid sequences related by the well-known base-pairing rules that A pairs with T and C pairs with G. Complementarity can be “partial,” in which only some of the nucleic acid bases are matched according to the base pairing rules. On the other hand, there may be “complete” or “total” complementarity when all of the bases are matched according to base pairing rules. The degree of complementarity between nucleic acid strands determines the efficiency and strength of hybridization therebetween.

Chromosome abnormalities are associated with genetic disorders and exposure to agents known to cause degenerative diseases, particularly cancer including hematological malignancies such as leukemia and lymphoma. The classic example of chromosome abnormality is the 9:22 translocation of the Philadelphia chromosome, which is associated with chronic myclogenous leukemia (CML).

As such, the ability to identity genetic rearrangements plays a central role in diagnosis and treatment of a variety of diseases.

Current methods for identifying genetic rearrangements include cytological techniques based upon chemical stains which produce a band pattern on a stained chromosome. Other, more advanced methods for gene mapping are based on the availability of large quantities of pure DNA and RNA fragments for probe generation.

Prior art detection methods suffer from several drawbacks including: lack of detection of abnormal chromosomes in less than ideal metaphase spread, impracticality in determining the frequency of abnormal cells in a complex tissue, lack of specificity in identifying sub-chromosomal regions, time needed for either karyotyping or preparing probe, lack of flexibility, inefficient detection of marker chromosomes and failure to paint chromosomes adequately while still observing landmarks for longitudinal differentiation. Furthermore many prior art approaches are still limited by the lack of appropriate probes.

As described hereinunder and in the Examples section which follows, the present invention provides a novel approach for systematically identifying genetic rearrangements.

Aside from large scale applicability the present method can be used to identify genetic rearrangements not only at the DNA level but at the RNA and protein levels as well. Thus, the present invention circumvents the need for condensed chromosomes as targets, which is particularly beneficial in cases where such target sequences cannot be easily obtained. Furthermore the ability to screen for genetic rearrangements at the RNA level, negates the need to generate chromosomal probes, thus significantly enhancing an otherwise time-consuming and laborious procedure.

The present invention is particularly useful in monitoring disease progression, even in cases where the chromosomal aberration represents a small subpopulation in a heterogeneous biological sample. In addition, the present invention systematically identifies specific chromosomal breakpoints (i.e., transition points) and as such can be used to determine a subject predisposition to a variety of chromosomally based disease syndromes.

Thus, according to one aspect of the present invention there is provided a method of identifying putative fusion transcripts.

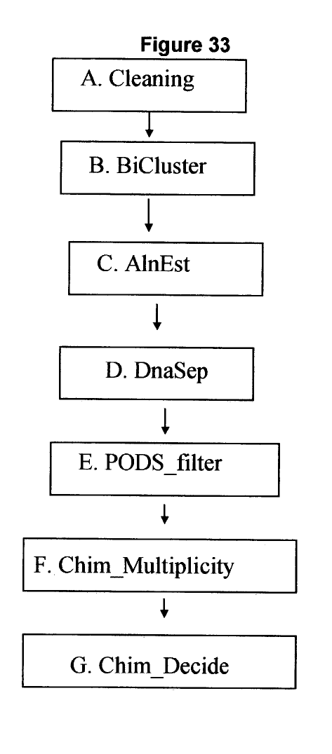

The method according to this aspect of the present invention is effected by the following steps (illustrated in FIG. 33).

First, annotated polynucleotide sequences of a first database are computationally aligned with a second database of expressed polynucleotide sequences.

Following computational alignment, expressed polynucleotide sequences, which exhibit complementarity to at least two non-contiguous sequences of the first database are identified.

The non-contiguous sequences can be non-homologous polynucleotide sequences mapped to different chromosomes, polynucleotide sequences mapped to different loci of a single chromosome and polynucleotide sequences mapped to a single locus and not being a part of cis-splice isoform

The expressed polynucleotide sequences, which include sequence regions which are complementary to the non-contiguous sequences, are considered as putative fusion transcripts and as such are preferably stored in a database which can be generated by a suitable computing platform.

The putative fusion transcripts identified can then be filtered to remove false-positive sequences using computational or laboratory approaches as is further described hereinbelow.

It will be appreciated that once fusion transcripts are identified and verified, sequence information derived therefrom can be used to characterize the transition points generated by the chromosomal/transcript rearrangements which produced such fusion transcripts.

Since such transition points are in fact markers for genetic rearrangements, their identification and characterization is important especially in case where such genetic rearrangements lead to, or are indicative of disorders.

In addition, sequencing of the fusion transcripts and identification of such transition points enables identification of the related polynucleotide sequences involved in the genetic rearrangements.

This is particularly important in cases where genetic rearrangements are associated with a disorder (e.g., cancer).

Accumulative knowledge shows strong correlation between a variety of human diseases and mutations, over-expression and function of the protein building blocks (i.e., protein kinases, phosphatsases) and their effectors and regulators, which constitute numerous intracellular signaling pathways For instance, inactivation of both copies of ZAP-70 or Jak-3 causes severe combined immunodeficiency and mutation of the X-linked BTK gene results in agammaglobulinemia. Many genetic disorders are also associated with mutations for example, in protein-serine kinases (PSKs) and phosphatases. The Coffin-Lowry syndrome results from inactivation of the X-linked Rsk2 gene, and myotonic dystrophy is due to decreased levels of expression of the myotonic dystrophy PSK. In addition, over-expression of ErbB2 receptor tyrosine kinase is implicated in breast and ovarian carcinoma [reviewed by Hunter T. (2000) Cell 100; 113-127].

Given the importance of activated kinases in a variety of disorders such as cancer, it would be anticipated that phosphatases regulation would be found as tumor suppressor genes and as promising drug targets. So far this has not proved to be the case. Furthermore, a number of diseases are associated with insufficient expression of signaling molecules, including non-insulin-dependent diabetes and peripheral neuropathies.

Since proteins participate in various cellular functions, identification of an open reading frame spanning at least one non-contiguous polynucleotide region within the identified fusion transcript (i.e., encompassing most if not all of the coding region of at least that polynucleotide region) can be used to determine the impact of a fusion event on cellular function. Identification of an open reading frame may be effected according to sequence parameters which are familiar to one skilled in the art. Identified polypeptides may be truncated (i.e., following introduction of a stop codon at transition point of the fusion transcript), non-functional or dysfunctional proteins (i.e., such as a fusion protein wherein a single open reading frame spans at least two non contiguous polynucleotide sequence regions) which can play a pivotal role in the development of disorders associated with genetic rearrangements.

Thus, it is conceivable that sequence information derived from the fusion transcripts identified according to the teachings of the present invention, can be used in a variety of clinical applications.

Probes representing transition, points can be used for assessing predisposition of individuals to a variety of disorders associated with genetic rearrangements, evaluating disease progression and/or response to treatment.

Identification of sequence regions and genes which are associated with tumor suppression or initiation/progression can also lead to the generation of therapeutic agents which can be used to suppress activity of newly formed oncogenes, or replace the lost activity of inactivated tumor suppressor genes.

The present invention can also be used for designing specific therapeutic agents against DNA, RNA, proteins or protein targets.

As mentioned hereinabove, the method according to this aspect of the present invention identifies the putative fusion transcripts in a database of expressed polynucleotide sequences.

Such a database can include sequences derived from libraries of expressed messenger RNA [i.e., expressed sequence tags (EST)], cDNA clones, contigs, pre-mRNA, which are prepared from specific tissues or cell-lines or from whole organisms.

This database can be a pre-existing publicly available database [i.e., GenBank database maintained by the National Center for Biotechnology Information (NCBI), part of the National Library of Medicine, and the TIGR database maintained by The Institute for Genomic Research] or private databases (i.e., the LifeSeq.™ and PathoSeq.™ databases available from Incyte Pharmaceuticals, Inc. of Palo Alto, Calif.).

Alternatively, the database can be generated from sequence libraries including, but not limited to, cDNA libraries EST libraries, mRNA libraries and the like.

Construction and sequencing of a cDNA library is one approach for generating a database of expressed mRNA sequences. cDNA library construction is typically effected by tissue or cell sample preparation, RNA isolation, cDNA sequence construction and sequencing.

It will be appreciated that such cDNA libraries can be constructed from RNA isolated from whole organisms, tissues, tissue sections, or cell populations. Libraries can also be constructed from a tissue reflecting a particular pathological or physiological state which is, for example, associated with genetic rearrangements.

Of particular interest are libraries constructed from hematological malignancies including leukemia and lymphoma Examples of such libraries include the human chronic myelogenous leukemia Creator™ Smart™ cDNA library and human acute myelogenous leukemia Creator™ Smart™ cDNA library (Cat. Nos. HL9507DD and HL9506DD, respectively Clontech Inc.).

As is mentioned hereinabove, the database of expressed polynucleotide sequences is computationally aligned with annotated polynucleotide sequences. As used herein the phrase “annotated polynucleotide sequences” refers to polynucleotide sequences, which are at least partially characterized with respect to functional or structural aspects of the sequence. Annotation can include identifying attributes such as locus name, intron/exon sequences, coding sequence, key words, Medline references and cloning data including tissue or cellular origin and pathological abundance.

Annotated polynucleotide sequences according to this aspect of the present invention may include genomic sequences, expressed sequence tags, contigs, complementary DNA (cDNA) sequences, pre-messenger RNA (e.g. hnRNA) sequences and mRNA sequences.

Preferably, the annotated database includes genomic sequences since the use of such sequences enables: (i) identification of intron/exon boundaries; (ii) detection of repetitive and low-complexity sequences; (iii) identification of chimeric sequences which result from genetic rearrangement at both DNA (i.e., chromosomal rearrangements) and RNA level (i.e., trans-splicing and the like).

Computational alignment of expressed polynucleotide sequences with annotated polynucleotide sequences can be effected using any commercially available alignment software including the local homology algorithm of Smith & Waterman, Adv. Appl. Math. 2,482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of algorithms GAP, BESTFIT, FASTA, and TFASTA in the Wisconsin Genetics Software Package Release 7.0, Genetics Computer Group, 575 Science Dr, Madison, Wis.

Another example of an algorithm which is suitable for sequence alignment is the BLAST algorithm, which is described iii Altschul et al., J. Mol. Biol. 215:403-410 (1990). Software for performing BLAST analyses is publicly available through the National Center for Biotechnology Information (http://www.nebi.nlm.nih.gov/).

Since, the present invention requires processing of large amounts of data sequence alignment is preferably effected using assembly software.

A number of commonly used computer software fragment read assemblers capable of forming clusters of expressed sequences, and aligning members of the cluster (individually or as an assembled contig) with other sequences (e.g., genomic database) are now available. These packages include but are not limited to, The TIGR Assembler [Sutton G. et al. (1995) Genome Science and Technology 1:9-19], GAP [Bonfield J K. et al. (1995) Nucleic Acids Res. 23:4992-4999], CAP2 [Huang X. et al. (1996) Genomics 33:21-31], The Genome Construction Manager [Laurence CB. Et al. (1994) Genomics 23:192-201], Bio Image Sequence Assembly Manager, SeqMan [Swindell S R. and Plasterer J N (1997) Methods Mol. Biol. 70:75-89], and LEADS and GenCarta (Compugen Ltd. Israel).

Once proper alignment is accomplished, a given expressed polynucleotide sequence exhibiting complementarity with more than one non-contiguous polynucleotide sequences is identified as a putative fusion transcript.

Concomitant with, or following computational alignment, identified putative fusion sequences are tested for the presence or absence of one or more sequence element in order to filter out false positive sequences.

Such filtering can be effected by computer assembly and alignment programs modified to incorporate sequence element criteria for determining chimerism.

The following section describes sequence elements and statistical parameters, which can be used for filtering-out false positive fusion transcripts and scoring true fusion events.

Alignment score (e-score)—E-scores represent the probability of a random sequence match within a given window of nucleotides. The lower the c-score, the better the match. One skilled in the art is familiar with e-scores (U.S. Pat. No. 6,221,587). Low alignment scores (i.e., e-scores) of expressed polynucleotide portions, within a fusion transcript, to non-contiguous polynucleotide sequences suggest that the fusion transcript is true.

Sequence conservation—Since human and nouse genes share high level of sequence conservation, fusion transcripts from human origin are aligned with the mouse genome. Fusion transcripts which exhibit complete sequence alignment with a signle mouse gene are poorly scored.

Gaps and assembly—Oftentimes genomic sequences are annotated as complete while other sequences are annotated as draft, suggesting that non-sequenced regions and gaps are included in the genomic segment.

The presence of gaps within a genomic sequence may indicate that the direction or order of the genomic segments is not accurate. Alternatively, gaps may indicate that the distance between two DNA fragments is unknown.

Examples of gap types are summarized hereinunder;

(i) Fragment—gaps between the contigs of a draft clone.

(ii) Clone—gaps between clones in the same map contig.

(iii) Contig—gaps between map contigs.

(iv) Heterochromatin—gaps from centromeres or other large blocks of heterochromatin.

It will be appreciated that in cases where the alignment of the fusion transcript to the genomic sequence is adjacent to a gap, the putative fusion transcript is poorly scored.

Low complexity sequences and interfering sequence repeats—These are either low complexity sequences or sequences composed of 2 to 10 base pairs repeated in long tandem repeats. Such sequences are often concentrated at certain regions of a chromosome. For example, in the mouse genome a large amount of simple sequence DNA is located near the centromeres. Because of their high abundance and repetitive nature these sequences may interfere with proper computational alignment and as such may contribute to the false identification of fusion transcripts.

Pseudogene sequences—Genomic sequence regions that do not result ill protein products, however they do exhibit sequence similarities to their true gene counterparts. Pseudogenes may arise from duplication of ancestral genes [Lodish et al., Molecular Cell Biology, 3rd. Ed. Scientific America, Inc., New York, N.Y. (1995)]. Typically, fusion transcripts which include pseudogenes are poorly scored.

Homologous sequences—Sequences which exhibit high levels of identity, and are mapped to different chromosomal loci should be distinguished from true fusion transcripts. Typically, homologous sequences are filtered-out.

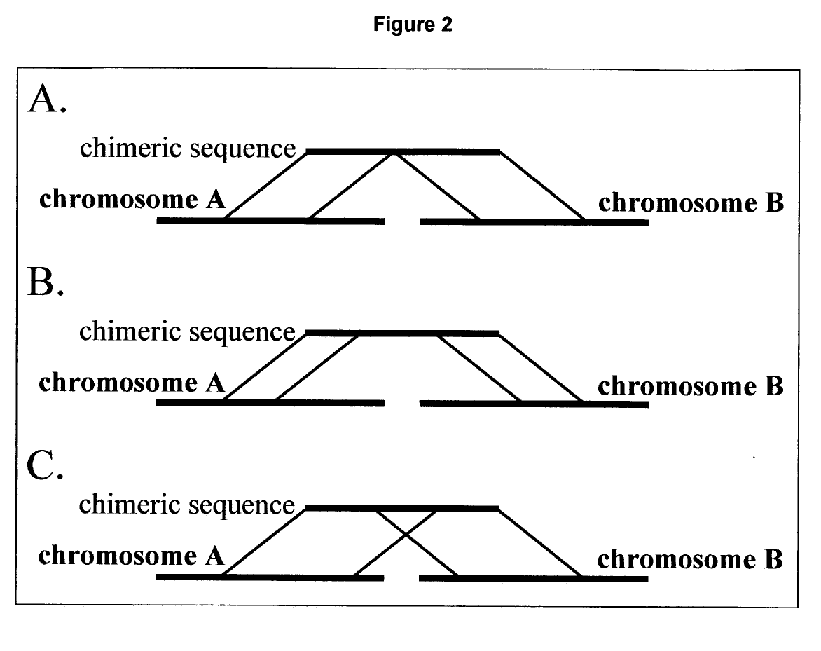

EST jump—Putative fusion transcripts we analyzed using the EST jump feature. The valvue of the EST jump is the distance in base pairs between the streaches of alignments on the chimeric sequence to the parental DNA sequences. The EST jump value may be indicative of the fusion event mechanism. The feature includes 3 ranges of values: positive EST-jump value indicative of insertions; negative EST-jump value indicative of a repetitive segment; zero EST jump value indicative of precise rejoining of the two portions which build-up the fusion transcript (see FIGS. 2a-c). Such analysis sheds light on the type of joining and fusion mechanisms (i.e., exon/exon, exon/intron, intron/intron, genomic region/exon and the like).

For example, fusion events resulting from exon shuffling show an EST-JUMP, which equals zero. Such EST-jump value supports the probability for a natural fusion event. Alternatively, positive EST jump value is indicative of nucleotide insertion at transition point. Such insertions may point to artificial fusion event, arising from for example, residual sequence tags ligated during library generation. In such case fusion transcripts with positive EST jump value are filtered-out. Alternatively, positive EST jump value may arise from natural mechanisms of rearrangement. For example V(D)J recombination involving deoxynucleotidil transferase activity, which adds non-template random sequences at the transition point. Fusion transcripts including V(D)J cleavage-rejoining sites at transition points are highly scored (Further description of V(D)J recombination is provided in the Background section). Negative EST-jump value is indicative of deletion of genomic fragment as compared to the parental polynucleotide sequences involved in the event. Negative EST-jump value may indicate on unequal recombination mechanisms (i.e., non-homologous recombination of homologous sequences).

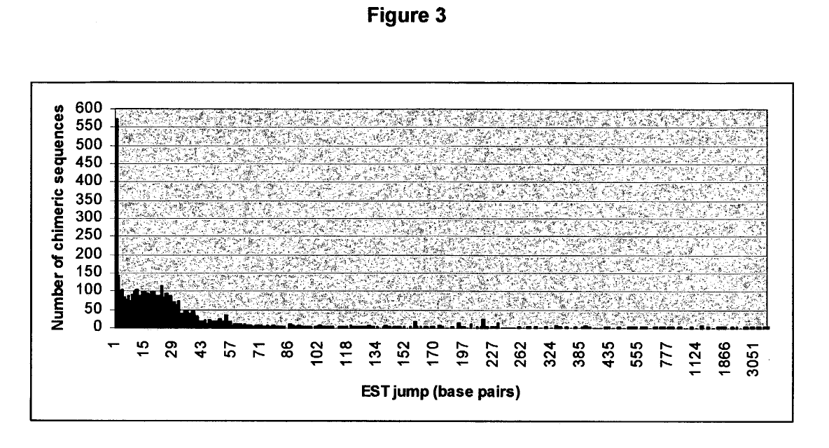

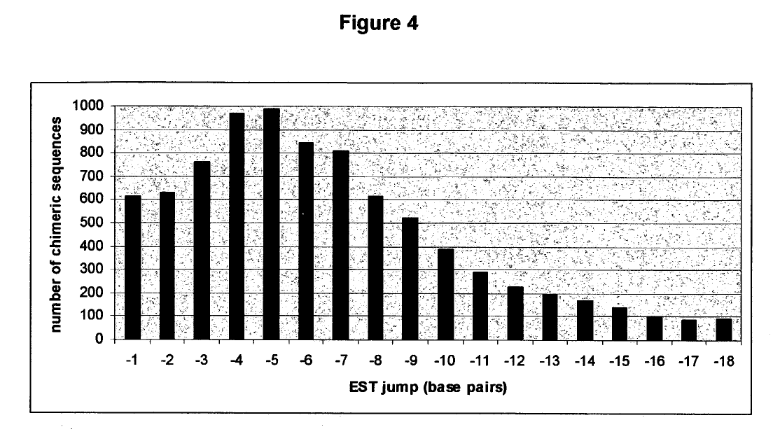

Chimeric fusion transcripts in the enclosed file chimeric_contigs_information.txt of the attached CD-ROM were analysed for EST jumps using the hereinabove described guidelines; Most of the positive EST jump values are below 55 (see FIG. 3), while most of the negative EST jump values are between −1 and −10 (sec FIG. 4). It will be appreciated that according to presently known configurations the negative EST jump values are limited to −18.

Sequences repeats—As described hereinabove low complexity sequences sets hurdles in the alignment process of the fusion transcript to the genome, therefore sequences containing such are frequently filtered out. However, in many cases sequence repeats are implicated in chromosomal rearrangement mechanisms (see for example Repeat Masker and Repbase databases). For example, LIES and SINES, recombination sequences Like V(D)J and CHI-like, other special sequences like Translin and Topoll sites, low complexity region like di and tri nucleotide repeats, AT hooks, 3 to 10 nucleotide repeats, telomere-like repeats, satellite repeats and palindromic sequences. The correlation between repeats and genomic rearrangements is well established in the scientific work (The role of Alu repeat clusters as mediators of recurrent chromosomal aberrations in tumors [Genes Chromosomes Cancer. 2002 October;35(2):97-112]. Sequences, which are proximate to the chimeric break points in both participating genes are analyzed. Sequence repeats may be indicative of the mechanism taking place in the chromosomal rearrangement event.

Expressed sequences distribution—number of expressed sequences supporting a putative fusion event. This analysis parameter may reflect fusion abundance. The higher the parameter value for a given fusion event the higher the probability for its natural existence.

Library distribution—The number of libraries supporting a putative fusion event. The higher the value the greater the probability for a true fusion event. It will be appreciated though that multiple fusion transcripts obtained from a single library are typically considered artificial and may reflect library quality.

Determination of chimeric ESTs number per library can be effected as follows:

1. Determination of the percentage of fusion transcripts in a library.

2. Determination of the actual number of fusion transcripts in a library.

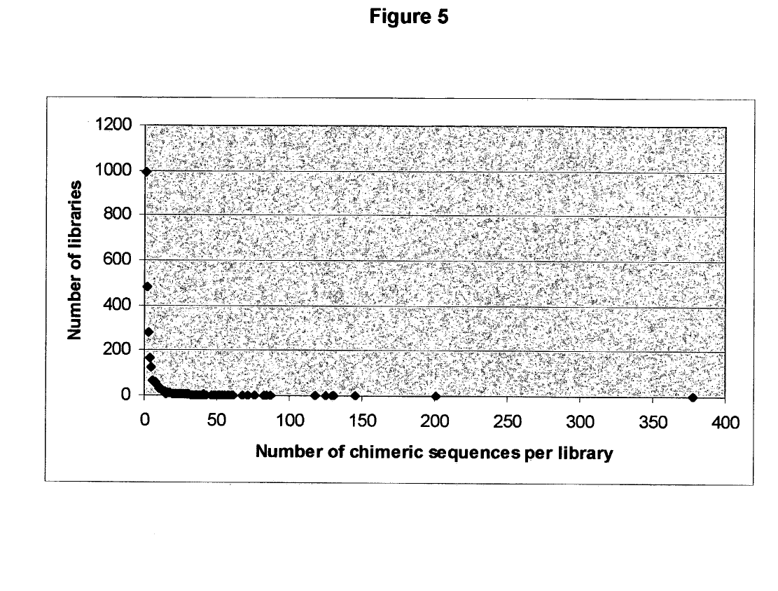

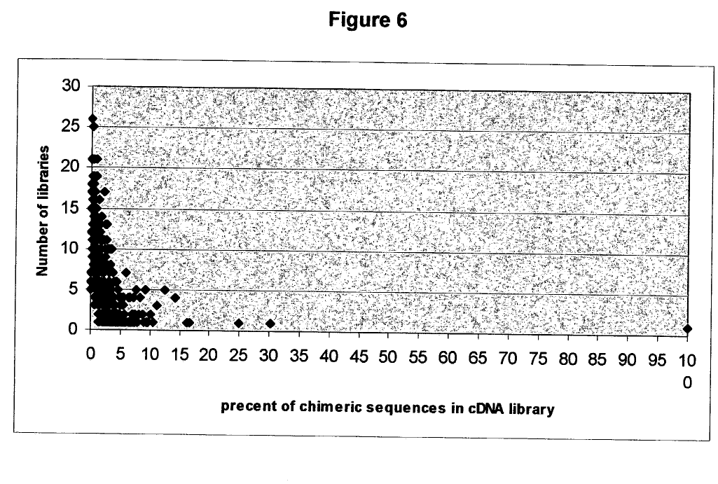

Fusion sequences arising from libraries with relative high number/percentage of chimeric sequences are poorly score. FIGS. 5 and 6 show the number of fusion sequences per library and the percentage of fusion sequences per library, respectively.

Spice consensus sites and intron length—Cis-splicing (splicing which occurs in the same gene) isoforms may be mistakenly identified as fission transcripts. In-order to distinct between splice isoforms and fusion transcripts, sequences considered as introns are defined by sequence length. As the majority of known introns are less than 400,000 bp long, ESTs aligned to two non-contiguous polynucleotide sequences separated by more than 400,000 bp are treated as true fused sequences, unless they exhibit consensus splice donor and acceptor sites. To reduce number of false negative fusion transcripts, a less stringent filter is typically used, which allows maximal intron length of 100,000. Thus, sequences up to 100,000 base pairs are considered as true introns. This test is accompanied by another parameter defining intron/exon boundaries (i.e. splice site consensus sequence), which is specifically valuable in cases where intron-length exceeds 100,000 base pairs. The 5′ splice site consensus sequence (i.e., “donor”) is AG/GURAGU (where A=adenosine, U=uracil, G=guanine, C-cytosine, R-purine and/=the splice site). The 3′ splice region consists of three separate sequence elements: the branch point or branch site, a polypyrimidine tract and the 3′ splice consensus sequence (i.e., “acccptor”) (YAG) (where A=adenosine, G=guanine, Y=pyrimidine). These elements loosely define a 3′ splice region, which may encompass 100 nucleotides of the intron upstream of the 3′ splice site. The branch point consensus sequence in mammals is YNYURAC (where N=any nucleotide, Y=pyrimidine). The underlined A is the site of branch formation. The 3′ splice consensus sequence is YAG/G. A polypyrimidine tract usually resides between the branch point and the splice site, this polypyrimidine tract is important in mammalian systems for efficient branch point utilization and 3′ splice site recognition [Roscigno, R., F. et al., (1993) J. Biol. Chem. 268:11222-11229]. The first YAG trinucleotide downstream from the branch point and polypyrimidine tract is the most commonly used 3′ splice site [Smith, C. W. et al., (1989), Nature 342:243-247].

Intron analysis is preferably effected in combination with the above-described analysis using various sequence elements.

Exonization—Fusion transcript which include intact exons are poorly scored, since it is more likely that the DNA will break inside an intron than inside an exon since introns are much longer than exons. Identification of exon boundaries is effected as described hereinabove in the “Splice consensus sites and intron length” section.

The probability that such a fusion event will occur randomly as an artifact during the creation of the expressed sequnece (i.e., the probability that one fragment of EST/RNA will end exactly in the end of an exon and the other fragment of EST/RNA that ligates to it stars exactly in the start of an exon) is: (Exon size of gene A)×(Exon size of gene B). Current estimation based on an average exon size is about 1:250²=1:62500.

Transcript length—Fusion transcripts exhibiting only short complementation (i.e., less than 50 bp) to non-contiguous polynucleotide sequences, are typically filtered-out. Such short complementation may arise from poor quality sequencing of expressed polynucleotide sequences.

Vector contaminant—Detection of vector sequences is performed in order to remove vector sequence used in library cloning. Vector sequences may cause an expressed sequence to mistakenly align with other sequences which are not related to the coding portion of the gene. Generally, filtering using vector sequence data is accomplished by comparing expressed sequence data with known vector sequences (e.g., Vector sequence database) and detecting sequences identical to the known vector sequences. Of note are vector sequences which are located at the vicinity of the transition point.

Tissue and pathological distribution—A putative fusion event showing strong correlation with a specific tissue or pathology such as type of cancer are highly scored.

Reciprocal fusion events—identification of a reciprocal event for a given fusion transcript, largely supports the natural existence of the fusion event and provides a preliminary mechanism such as for example reciprocal translocation (see Background section).

Restriction sites—Putative fusion transcripts are scanned for restriction site sequences at the vicinity of the transition point (i.e., 40 base pairs upstream or downstream to the transition point) from the sides of the breakpoint and the regions defined as positive EST-JUMP). Cases in which such restriction sequences were used during library construction are filtered out. For example, blunt restriction enzymes which are blunt restriction endonucleases (e.g., SspI, AluI, ScaI, SmaI, HincI and DraI, commercially available from MBI Fermentas) generate blunt-end DNA fragments as opposed to protruding end endonucleases. Blunt end restriction enzymes are occasionally used when cloning DNA fragments during the preparation of DNA libraries. Because of their structural nature, these fragments may be incorrectly joined to form artificial chimeras. Hence, putative fusion transcript are tested for blunt-end restriction sequences which may have been used during the generation of expressed sequence libraries. Typically, fusion transcripts which include such sequence elements at transition point are filtered out.

Overhang lengths—refers to the distance between the transition point sequence of a putative fusion transcript and the end of the available polynucleotide sequence aligned thereto. Short overhanging genomic sequences may suggest that the putative fusion transcript has been identified due to lack of annotated sequence information. For example, a short overhanging sequence may be an intron sequence, whose successive exon sequence may be unavailable is yet in accessible databases. In such case, alignment of the alleged fusion transcript to a second non-contiguous polynucleotide sequence may be artificial and proceed for example, due to sequence homology or repeated sequences. Typically fusion sequences showing short overhangs are filtered-out.

Sequence directionality—Fusion transcripts which exhibit two directional splicing, such that the canonical splice site GT-AG, GC-AG and AT-AC is identified on one sequence end and the opposite sequences CT-AC, CT-GC and GT-AT is identified on the other end are considered artifacts and are filtered out.

Events per library—Single genes which participate in more than one fusion event in a single library can be explained by tumo heterogeneity, however such sequences may also be artifactual and thus are poorly scored.

Examples of the above described filtering guidelines is presented in Examples 2-7 of the Examples section which follows.

Following identification of fusion transcripts, transcript sequences and annotation signifying their abundance (i.e., including tissue abundance, pathological abundance), chromosomal location, EST-jump value, hotspot sequences, length, parental polynucleotide sequences related thereto and the like are stored in a database for further use (see FIGS. 57a and 57b, example 13).

It should be noted that since annotation of publicly available databases is at times unreliable, assessment of pathological abundance can only be effected in cases where the identity of the source tissue of a publicly available database can be confirmed

Several other computational approaches routinely used in the art can be used in addition, or as an alternative to, the above described computational screening approach.

One such procedure is “electronic northern analysis”. In general, an electronic northern has two objectives: to determine the libraries in which a given transcript is expressed, and to determine abundance levels of expression in the libraries in which it is expressed. An analysis of the levels of transcript expression is performed using the transcript image of each library or sample examined. The abundance of the expression is then shown for each selected sample. The electronic northerns mimic conventional “wet lab” northerns done in a laboratory in that they allow comparative analysis of levels of expression of a single gene or gene family. Electronic northerns can be performed for different tissue types of a single source, for the same tissue type from different sources (e.g. to develop a standard for normal expression), for the same tissue type of a single species at different stages of development (i.e. an electronic developmental Northern), for the same tissue type across species (e.g. evolutionary studies), and for normal and abnormal samples derived from the same tissue type (e.g. normal tissue versus cancerous tissue). Expression may give insight on the timing of expression, potential function of the gene product, and involvement in the disease state.

Further or alternative validation of computationally obtained putative fusion transcripts can be effected using various laboratory approaches such as, for example, FISH analysis, PCR, RT-PCR, southern blotting, northern blotting, electrophoresis and the like (see Examples 11-12 of the Examples section). Such screening methodologies are preferably executed under physiological conditions (i.e., temperature, pH, ionic strength, viscosity, and like biochemical parameters which are compatible with a viable organism, and/or which typically exist intracellularly in a viable cultured yeast cell or mammalian cell).

Although the present methodology can be effected using prior ant systems modified for such purposes, due to the large amounts of data processed and the vast amounts of processing needed, the present methodology is preferably effected using a dedicated computational system.

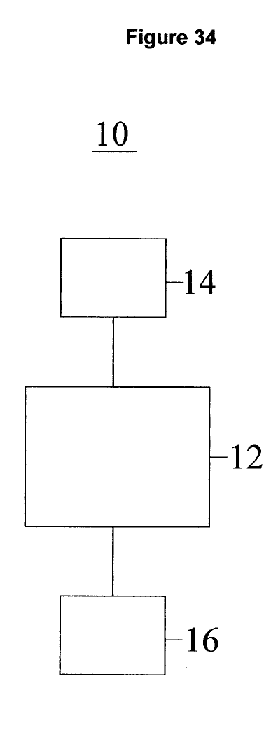

Thus, according to another aspect of the present invention and as illustrated in FIG. 34, there is provided a system for generating a database of sequence information of at least a portion of the putative fusion transcripts which system is referred to hereinunder as system 10.

System 10 includes a processing unit 12, which executes a software application designed and configured for identifying putative fission transcripts as described hereinabove. System 10 further serves for filtering out false positive sequences as described hereinabove and optionally storing the sequences of fusion transcripts as a retrievable/searchable database. Such a database father includes information pertaining to database generation (e.g., source library), parameters used for selecting polynucleotide sequences, putative uses of the stored sequences, and various other annotations and references which relate to the stored sequences or respective sense transcripts.

System 10 may also include a user input interface 14 (e.g., a keyboard and/or a mouse) for inputting database or database related information, and a user output interface 16 (e.g., a monitor) for providing database information to a user.

System 10 preferably stores sequence information of the putative fusion transcripts identified thereby on a computer readable media such as a magnetic, optico-magnetic or optical disk to thereby generate a database of putative fusion transcript sequences.

System 10 of the present invention may be used by a user to query the stored database of sequences, to retrieve nucleotide sequences stored therein or to generate polynucleotide sequences from user inputted sequences.

System 10 can be any computing platform known in the art including but not limited to, a personal computer, a work station, a mainframe and the like.

It will be appreciated that system 10 can be modified to generate a database of sequence information pertaining specifically to the transition point nucleic acid sequences of the putative fusion transcript.

The database generated and stored by system 10 can be accessed by an on-site user of system 10, or by a remote user communicating with system 10.

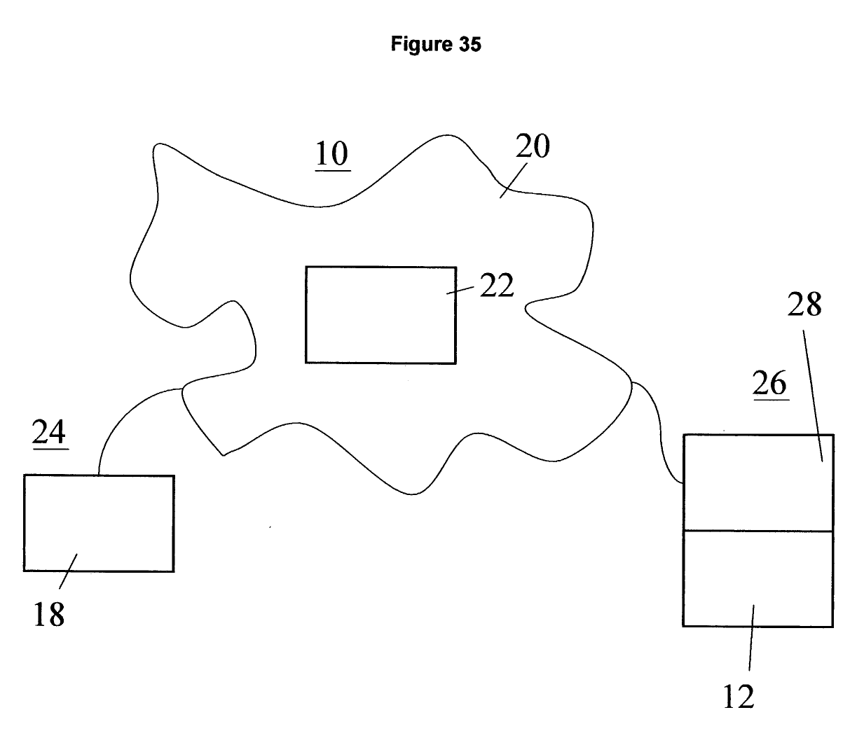

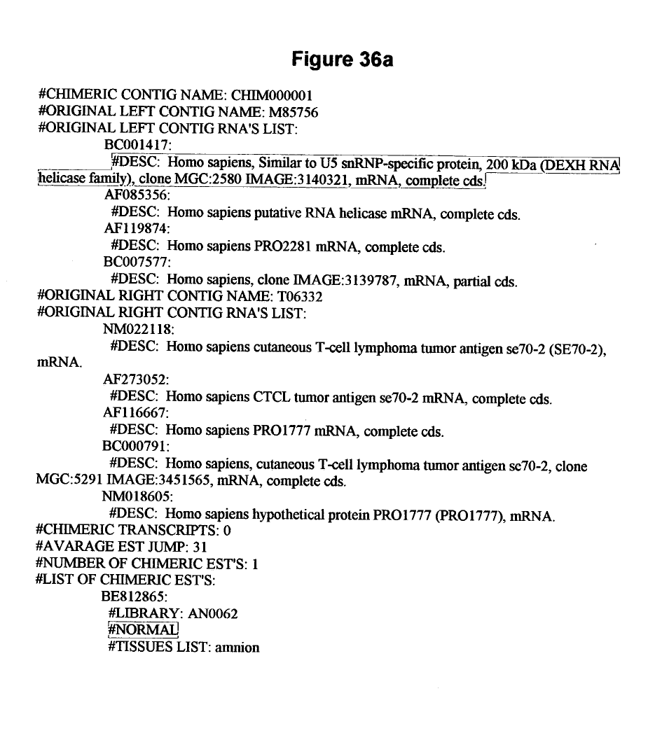

As illustrated in FIG. 35, communication between a remote user 18 and processing unit 12 is preferably effected via a communication network 20. Communication network 20 can be any private or public communication network including, but not limited to, a standard or cellular telephony network, a computer network such as the Internet or intranet, a satellite network or any combination thereof.

As illustrated in FIG. 35, communication network 20 includes one or more communication servers 22 (one shown in FIG. 35) which serves for communicating data pertaining to the sequence of interest between remote user 18 and processing unit 12.

It will be appreciated that existing computer networks such as the Internet can provide the infrastructure and technology necessary for supporting data communication between any number of sites 24 and remote analysis sites 26.

For example, using a computer operating a Web browser application and the World Wide Web, any expressed polynucleotide sequence of interest can be “uploaded” by user 18 onto a Web site maintained by a database server 28. Following uploading, database server 28 which serves as processing unit 12 can be instructed by the user to processes the polynucleotide as is described hereinabove.

Following such processing, which can be performed in real time, nucleic acid sequence results can be displayed at the web site maintained by database server 28 and/or communicated back to site 24, via for example, e-mail communication.

Thus, using the Internet, a remote configuration of system 10 can provide polynucleotide sequence analysis services to a plurality of sites 24 (one shown in FIG. 35).

As is mentioned hereinabove, the sequence data extracted according to the teachings of the present invention is highly valuable, since it enables generation of oligonucleotide probes or primers, which can specifically hybridize to genetically rearranged sequences.

The oligonucleotide probes of the present invention preferably comprise nucleic acid sequences that are substantially homologous to nucleic acid sequences that flask and/or extend across transition points, which are associated with the genetic rearrangements.

Oligonucleotides generated by the teachings of the present invention may be used in any modification of nucleic acid hybridization based techniques. As such the oligonucleotides of the present invention can correspond to any cDNA, mRNA and genomic sequences for stretches between about 10 bp to about 20 or to about 30 base pairs (bp), with even longer sequences including 40, 50, 100 bp, or even longer. Oligonucleotides of 10 to 1000 or so bp or even more may have utility as hybridization probes in a variety of hybridization techniques including Southern and Northern blotting. The total size of oligonucleotide used, as well as the size of complementary sequences depend on the intended use or application of the reagent. It will be appreciated that larger sized DNA fragments (i.e., 5 kb, 10 kb. 20 kb, 30 kb, 50 kb) may also be used as probes such as in chromosomal painting techniques (e.g., FISH analysis).

In general, the oligonucleotides of the present invention may be generated by any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and as such is not further described herein.

The oligonucleotides of the present invention may comprise heterocylic nucleosides consisting of purines and the pyrimidines bases, bonded in a 3′ to 5′ phosphodiester linkage.

Preferably used oligonucleotides are those modified in either backbone, internucleoside linkages or bases, as is broadly described hereinunder. Such modifications can oftentimes facilitate oligonucleotide uptake and resistivity to intracellular conditions.

Specific examples of preferred oligonucleotides useful according to this aspect of the present invention include oligonucleotides containing modified backbones or non-natural internucleoside linkages. Oligonucleotides having modified backbones include those that retain a phosphorus atone in the backbone, as disclosed in U.S. Pat. Nos. ,67,808; 4,469,863; 4,476,301; 5,023,243, 5,177,196; 5,188,897; 5,264,423; 5,276,019; 5,278,302, 5,286,717; 5,321,131; 5,399,676; 5,405,939; 5,453,496; 5,455,233; 5,466,677; 5,476,925; 5,519,126; 5,536,821; 5,541,3065,550,111; 5,563,253; 5,571,799; 5,587,361; and 5,625,050.

Preferred modified oligonucleotide backbones include, for example, phosphorothioates chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylpliosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones include backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides which can be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic, includes peptide nucleic; acid (PNA). A PNA oligonucleotide refers to an oligonucleotide where the sugar-backbone is replaced with an amide containing backbone, in particular an aminoethylglycine backbone. The bases are retained and are bound directly or indirectly to aza nitrogen atoms of the amide portion of the backbone. United States patents that leach the preparation of PNA compounds include, but are not limited to, U.S. Pat. Nos. 5,539,082; 5,714,331; and 5,719,262, each of which is herein incorporated by reference. Other backbone modifications, which can be used in the present invention are disclosed in U.S. Pat. No. 6,303,374.

Oligonucleotides of the present invention may also include base modifications or substitutions. As used herein, “uwimodified” or “natural” bases include the purine bases adenine (A) and guanine (CT), and the pyrimidine bases thymine (T), cytosine (C) and uracil. (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanne, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-myethladenine, 8-azaguanine and 8-azaadenine, 7-deazaguanine and 7-deazaadeninc and 3-deazaguanine and 3-deazaadenine. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Such bases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi Y S et al. (1993) Antisense Research and Applications, CRC Press, Boca Raton 276-278] and are presently preferred base substitutions, even more particularly when combined with 2′-O-methoxyethyl sugar modifications.

Another modification of the oligonucleotides of the invention involves chemically linking to the oligonucleotide one or more moieties or conjugates, which enhance the activity, cellular distribution or cellular uptake of the oligonucleotide. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety, as disclosed in U.S. Pat. No. 6,303,374.

It is not necessary for all positions in a given oligonucleotide molecule to be uniformly modified, and in fact more than one of the aforementioned modifications may be incorporated in a single compound or even at a single nucleoside within an oligonucleotide.

The oligonucleotides of the present invention may be widely used as diagnostic, prognostic and therapeutic tool in a variety of disorders which are associated with genetic rearrangements. Examples of disorders-associated with genetic rearrangements include but are lot limited to acute lymphoblastic leukemia (ALL), acute myeloid leukemia (AML), chronic myelogenous leukemia Burkitt's lymphoma, adenocarcinoma, follicular lymphoma, Wilm's syndrome, plasma cell dyscrasias, rhabdomyosarcoma, osteosarcoma, refractory anemia velo-cardio-facial syndrome (VCF) DiGeorge syndrome. Further details on genetic disorders-associated diseases are disclosed in the Mitelman database http://cgap.nci.nih.gov/Chromosomes/Mitelamn, which is fully incorporated herein.

Oligonucleotides generated according to the teachings of the present invention can be included in diagnostic kits. For example, oligonucleotides sets pertaining to specific disease related fusion transcripts can be packaged in a one or more containers with appropriate buffers and preservatives along with suitable instructions for use and used for diagnosis or for directing therapeutic treatment.

Preferably, the containers include a label. Suitable containers include, for example, bottles, vials, syringes, and test tubes. The containers may be formed from a variety of materials such as glass or plastic.

In addition, other additives such as stabilizers, buffers, blockers and the like may also be added.

Oligonucleotides of the present invention can be particularly useful in the analysis of a subject's predisposition to a disorder which is associated with genetic rearrangements (e.g., cancer).

Predisposition to disorders-associated with genetic rearrangements can be ascertained by testing any tissue of a human for the presence or absence of such genetic rearrangements.

For example, a person who has inherited germline Multiple Tumor Suppressor (MTS) gene rearrangements would be prone to develop cancers [Cayuela J M et al. (1996) Blood 87(6):2180-6]. This can be determined by testing DNA from any tissue of the pcrson's body. Most simply, blood can be drawn and DNA extracted from the blood cells. In addition, prenatal diagnosis can be accomplished by testing fetal cells, placental cells or amniotic fluid for mutations of the MIS gene.

Present-day predisposition analysis tests are performed by chromosomal painting techniques. Notably, not only do these assays require condensed metaphase chromosomes which are occasionally very difficult to obtain, but these methods do not take into consideration genetic rearrangements that take place during the transcriptional process. Negating transcriptional rearrangements can often lead to inaccurate analysis and put a subject in unnecessary risk of disease development.

The present invention provides a method of identifying predisposition to disorders associated with genetic rearrangements, which is devoid of the above-limitations.

Thus, according to another aspect of the present invention there is provided a method detecting nucleic acid sequence chimerism indicative of predisposition for disorders associated with genetic rearrangements in a subject. As used herein a “subject” refers to a mammal such as a canine, a feline, an ovine, a porcine, an equine, or a bovine; preferably the term “subject” refers to a human.

The method according to this aspect of the present invention is effected by determining the presence or absence of a chimeric transcript in tissue of the subject. This can be effected by contacting a biological sample obtained from the subject with at least one oligonucleotide which is complementary to at least one transition point of a chimeric transcript.

Contacting the oligonucleotides of the present invention with the biological sample is effected by stringent, moderate or mild hybridization (as used in any polynucleotide hybridization assay such as northern blot, dot blot, RNase protection assay, RT-PCR and the like). Wherein stringent hybridization is effected by a hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 1-1.5° C. below the Tₘ, final wash solution of 3 M TMACI, 0.01 M sodium phosphate (pH 6.8), 1 mM EDTA (pH. 7.6), 0.5% SDS at 1-1.5° C. below the Tₘ; moderate hybridization is effected by a hybridization solution of 6×SSC and 0.1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 2-2.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ, final wash solution of 6×SSC, and final wash at 22° C.; whereas mild hybridization is effected by a hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 37° C., final wash solution of 6×SSC and final wash at 22° C.

It will be appreciated that the technique of Marin and Henegariu et al “specific multiplex PCR” [(2001) Haematologica 86:1254-1260; and (1997) Biotechniques 23:504-511): may be preferably implemented when practicing the above-described method of this aspect of the present invention.

Diagnostic oligonucleotides prepared according to the teachings of the present invention can be attached to a solid substrate, which may consist of a particulate solid phase such as nylon filters, glass slides or silicon chips [Schena et al. (1995) Science 270:467-470].

In a particular embodiment, oligonucleotides prepared according to the teachings of the present invention can be attached to a solid substrate configured as a microarray. Microarrays are known in the art and consist of a surface to which probes that correspond in sequence to gene products (e.g., cDNAs, mRNAs, cRNAs, polypeptides, and fragments thereof), can be specifically hybridized or bound at a known position (regiospecificity).

Several methods for attaching the oligonucleotides to a microarray are known in the art including but not limited to glass-printing, described generally by Schena et al., 1995, Science 270:467-47, photolithographic techniques [Fodor et al. (1991) Science 251:767-773], inkjet printing, masking and the like.

In general, quantifying hybridization complexes is well known in the art and may be achieved by any one of several approaches. These approaches are generally based on the detection of a label or marker such as any radioactive, fluorescent, biological or enzymatic tags or labels of standard use in the art. A label can be applied on either the oligonucleotide probes or nucleic acids derived from the biological sample.

The following illustrates a number of labeling methods suitable for use in the present invention. For example, oligonucleotides of the present invention can be labeled subsequent to synthesis, by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent. Alternatively, when fluorescently-labeled oligonucleotide probes are used, fluorescein, lissamine, phycoerythrin, rhodamine (Perkin Elmer Cetus), Cy2, Cy3, Cy3.5, Cy5, Cy5.5, Cy7, Fluor X (Amersham) and others [e.g., Kricka et al. (1992), Academic Press San Diego, Calif] can be attached to the oligonucleotides. It will be appreciated that pairs of fluorophores are chosen when distinction between two emission spectra of two oligonucleotides is desired or optionally, a label other than a fluorescent label is used. For example, a radioactive label, or a pair of radioactive labels with distinct emission spectra can be used [Zhao et al. (1995) Gene 156:207]. However, because of scattering of radioactive particles, and the consequent requirement for widely spaced binding sites, the use of fluorophores rather than radioisotopes is more preferred.

The intensity of signal produced in any of the detection methods described hereinabove may be analyzed manually or using hardware and software suited for such purposes.

In general, transcript quantification is preferably effected alongside a calibration curve so as to enable accurate mRNA determination. Furthermore, quantifying transcript(s) originating from a biological sample is preferably effected by comparison to a normal sample, which is characterized by normal expression pattern of the examined transcript(s).

Oligonucleotides generated according to the teachings of the present invention may also be used in screening for novel mutagenic agents.

Environmental and genetic causes underlic the malignant process. It is estimated, though that from about 70 to about 90 percent of cancer cases are linked to environmental carcinogens. Epidemiologists estimate that most cases of cancer would be preventable if the main risk and anti-risk factors could be identified. One epidemiological example of this phenomenon is colon and breast cancer, which constitute the major types of cancer, but are quite rare among Japanese living in Japan, while highly abundant with Japanese living in the United States.

To date, millions of chemical compounds are synthesized and a large portion of which are in commercial production and in economic use. There is thus a need to be able to determine which of these new compounds is carcinogenic.

The most reliable means for determining whether a particular compound is carcinogenic is a long term assay, which is based on the experimental assessment of the potential of the substance to induce tumors in rodents. These long term assays usually take 6 to 12 months to conduct, and they are relatively expensive. Because of the extended time periods and the expense involved, it is not feasible to conduct long term assays for high through put screening. The need for relatively fast and inexpensive means for preliminarily evaluating the cancer-causing potential of new chemicals has led to the development of relatively rapid screening assays; some of these are described in U.S. Pat. No. 4,701,406, the disclosure of which is hereby incorporated by reference.

The most widely known of these short-term assays is the Ames Assay [Ames et al., “Methods for Detecting Carcinogens and Mutagens with the Salmonella/Mammalian-Microsome Mutagenicity lest,” Mutation Research, vol. 31 (1975), pp. 347-364]. A major disadvantage of the Ames Assay is that many classes of carcinogenic compounds consistently show poor responses in this assay and also in mammalian cell genotoxic assay systems.

The present invention provides a rapid and easy approach for systematically identifying potential mutagenic agents (e.g., carcinogens). This method screens for genetic rearrangements, which are frequently associated wilt a variety of disorders specifically, cancer.

The method of this aspect of the present invention is effected by exposing cells to a plurality of putative mutagen is and determining which mutagen is capable of inducing expression of a chimeric transcript, thereby identifying a potential mutagenic agent. Identification of expression of a chimeric transcript is accomplished by hybridization with at least one oligonucleotide generated according to the teachings of the present invention.

Additionally, the method according to this aspect of the present invention is capable of identifying somatic mutations in the parental polynucleotides participating in the mutagen induced fusion events.

Putative mutagens or mutagenic agents, which can be utilized according to the present invention include, can include small molecules, such as, for example, naturally occurring compounds (e.g., compounds derived from plant extracts, microbial broths, and the like) or synthetic organic or organometallic compounds; mutagenic agents can also include viruses and microorganisms such as bacteria and intracellular parasites.

Various growth conditions can also be used as putative mutagens. Conditions suitable for use as putative mutagens according to the present invention include, but are not limited to, temperature, humidity, atmospheric pressure, gas concentrations, growth media, contact surfaces, radiation exposure (such as, gamma radiation, UV radiation, X-radiation) and the presence or absence of other cells in a culture.

Various cell types can be used by the present mutagen screening method. Examples include cells such as fibroblasts, epithelial cells, endothelial cells, lymphoid cells, neuronal cells, etc. Such cells should be readily propagatable in culture. Specific examples thus include, but are not limited to, various cell lines such as 293T, NIH, 3T3, H5V and L-cells, etc (All available from.

The method of this aspect of the present invention identifies expression of fusion transcript as indicator of genetic rearrangements. To this end measures are taken to maintain cell viability following exposure to the putative mutagenic agent, such that transcriptional activity is retained.

It will be appreciated that the teachings of the present invention can also be used for other purposes such as design of specific therapy against “tumor-specific” DNA, mRNA, proteins or protein targets, which are identified according to the teachings of the present invention.

Therapeutic agents useful against such targets are still quite scarce, however their therapeutic significance has been substantiated in the case of STI-571, a specific BCR-ABL tyrosine kinase inhibitor. The fused BCR-ABL gene product of the Philadelphia chromosome is characterized by a deregulated tyrosine kinase activity. STI-571 occupies the kinase pocket of the BCR-ABL protein and blocks ATP binding thereby preventing substrate phosphorylation [Druker B J et al (2001) N. Eng. J. Med. 344:1083-1042].

Nucleic acid sequences identified according to the teachings of the present invention can be employed in recombinant expression systems, such as for the production of antisense molecules, which can be used as therapeutic agents using standard gene therapy protocols. Antisense molecules, preferably directed at an active portion of the fusion transcript are inserted into a suitable vector. The vector is selected based on its ability to generate high levels of antisense RNA in conjunction with host cell machinery.

Functionally aberrant (i.e., non-functional, dysfunctional) truncated, mutated or fusion polypeptides and/or fragments thereof encoded from the fusion transcripts of the present invention may be used to prepare antibodies generated by standard methods. The antibodies may be polyclonal, monoclonal, recombinant, chimeric, single-chain and/or bispecific. Typically, the antibody or fragment thereof will either be of human origin, or will be “humanized”, i.e., prepared so as to prevent or minimize an immune reaction to the antibody when administered to a patient. The antibody fragment may be any fragment that is reactive with the TRIP1 of the present invention, such as, Fab, Fab′, etc.

Antibodies may be used therapeutically, such as to inhibit binding of the aberrant polypeptide to an effector thereof. The antibodies may further be used for in vivo and in vitro diagnostic purposes, such as in labeled form to detect the presence of the polypeptide directed thereto in a biological sample.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531. 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4.098,876; 4,879,219; 5,011,771 and 5,281,521; “Oligonucleotide Synthesis” Gait, M. J., ed. (1984); “Nucleic Acid Hybridization” flames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B. (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Terminology

“Chimeric event”—chimera that is descendent of fusion between two DNA loci (i.e., in the accuracy level of a single nucleotide);

“Total”—referring to all data in a given analysis.

“Translocations”—chimeric events between different chromosomes.

“Deletions”—chimeric events on the same chromosome between DNA fragments which are distant by at least. 100,000 base pairs.

“contig”—a gene or a part of a gene.

“contig—contig”—chimeric event—a case in which both loci taking part in the fusion event hits a contig.

“no contig—no contig”—chimeric event—a case in which both loci take part in the fusion event alings with an intergenic region.

“contig—no contig”—chimeric event—a case in which one of the loci taking part in the chimeric event alings with a gene and the other aligns with an intergenic region.

### Example 1

### Database Characterization

General Description of the Distribution of Chimeric Sequences and Chimeric Events in the Database—

(a) Total number of chimeric sequences: 13660

(i) ESTs: 12537

(ii) mRNAs: 1123

(b) Number of total participating contigs in the data: 10110

(c) Number of chimeric events: 11295

(i) Number of contig-contig cases: 6189

(ii) Number of contig-no contig cases 4069

(iii) Number of no-contig-no-contig cases: 1027

Distribution of sequences supporting chimeric events (Degree type 0)—The distribution of chimeric sequences supporting total chimeric events is illustrated in Table 1 below, wherein the column “#Sequences” denotes the number of chimeric sequences supporting the chimeric event and the column. “Events” denotes the number of chimeric events.

TABLE 1

| # Sequences | # Events |
|---|---|
| 1 | 9988 |
| 2 | 966 |
| 3 | 173 |
| 4 | 62 |
| 5 | 26 |
| 6 | 20 |
| 7 | 9 |
| 8 | 7 |
| 9 | 4 |
| 10 | 4 |
| 11 | 2 |
| 12 | 4 |
| 13 | 2 |
| 14 | 2 |
| 17 | 4 |
| 19 | 1 |
| 20 | 2 |
| 22 | 1 |
| 24 | 1 |
| 25 | 2 |
| 28 | 1 |
| 31 | 2 |
| 40 | 1 |
| 51 | 1 |

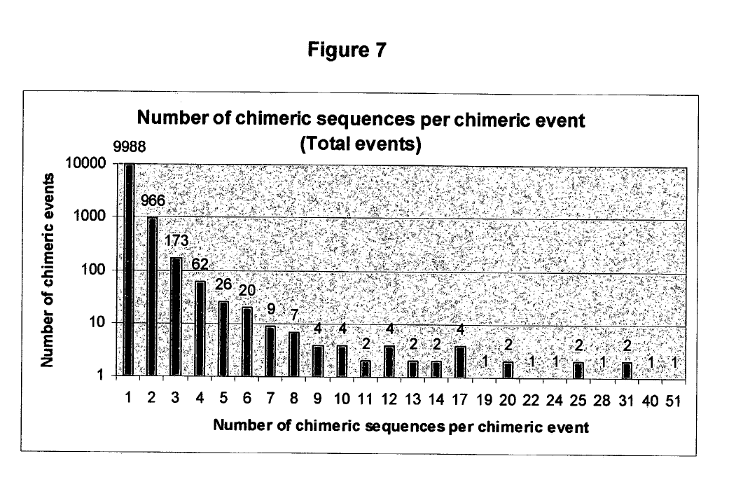

The distribution of chimeric sequences supporting total chimeric events is further illustrated in the graph of FIG. 7.

The distribution of chimeric sequences which result from translocations is illustrated in Table 2, below, wherein the column “#Sequences” denotes the number of chimeric sequences supporting the chimeric event and the column “Events” denotes the number of chimeric events.

TABLE 2

| # Sequences | # Events |
|---|---|
| 9165 | 1 |
| 838 | 2 |
| 144 | 3 |
| 40 | 4 |
| 20 | 5 |
| 10 | 6 |
| 2 | 7 |
| 4 | 8 |
| 3 | 9 |
| 2 | 10 |
| 2 | 11 |
| 2 | 17 |
| 1 | 20 |
| 1 | 25 |
| 1 | 28 |
| 1 | 31 |
| 1 | 40 |

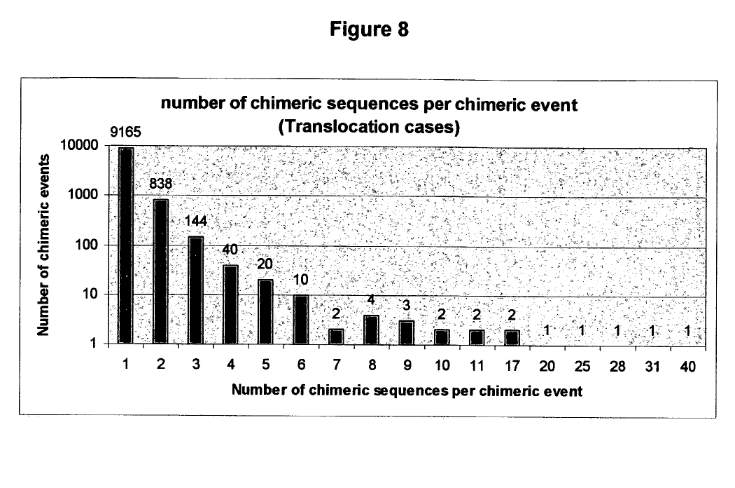

The distribution of chimeric sequences which result from translocations is further illustrated in FIG. 8.

The distribution of chimeric sequences which result from deletion is illustrated in Table 3, below wherein the column “#Sequences” denotes the number of chimeric sequences supporting the chimeric event and the column “Events” denotes the number of chimeric events.

TABLE 3

| # Sequences | # Events |
|---|---|
| 823 | 1 |
| 128 | 2 |
| 29 | 3 |
| 22 | 4 |
| 6 | 5 |
| 10 | 6 |
| 7 | 7 |
| 3 | 8 |
| 1 | 9 |
| 2 | 10 |
| 4 | 12 |
| 2 | 13 |
| 2 | 14 |
| 2 | 17 |
| 1 | 19 |
| 1 | 20 |
| 1 | 22 |
| 1 | 24 |
| 1 | 25 |
| 1 | 31 |
| 1 | 51 |

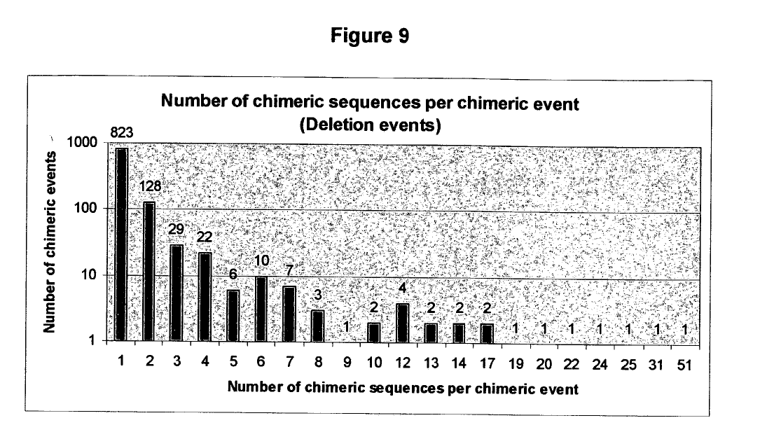

The distribution sequences which result from deletion is further illustrated in FIG. 9.

Distribution of libraries supporting chimeric events (Degree type 0)—The distribution of the cDNA libraries which support the total chimeric events is illustrated in Table 4, below, wherein the column “#Libraries” denotes the number of cDNA libraries which supporti a chimeric event and the column “#Events” denotes the number of chimeric events. Note that in the preparation of the data every sequence which was annotated as mRNA in Genebank was considered as originating from a different library. In addition, un-annotated sequences which did not include library origin were not considered.

TABLE 4

| # Libraries | # Events |
|---|---|
| 10816 | 1 |
| 345 | 2 |
| 56 | 3 |
| 23 | 4 |
| 8 | 5 |
| 12 | 6 |
| 3 | 7 |
| 3 | 8 |
| 4 | 9 |
| 4 | 10 |
| 1 | 12 |
| 2 | 13 |
| 3 | 14 |
| 1 | 16 |
| 3 | 18 |
| 1 | 24 |

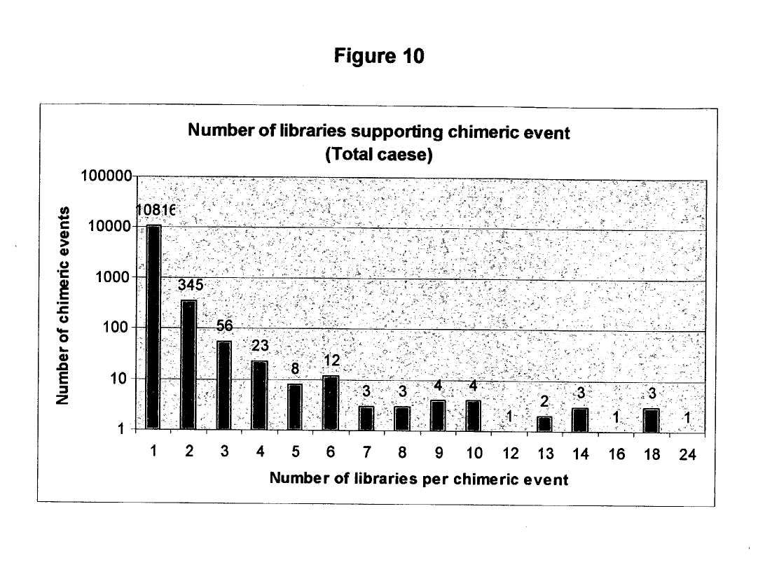

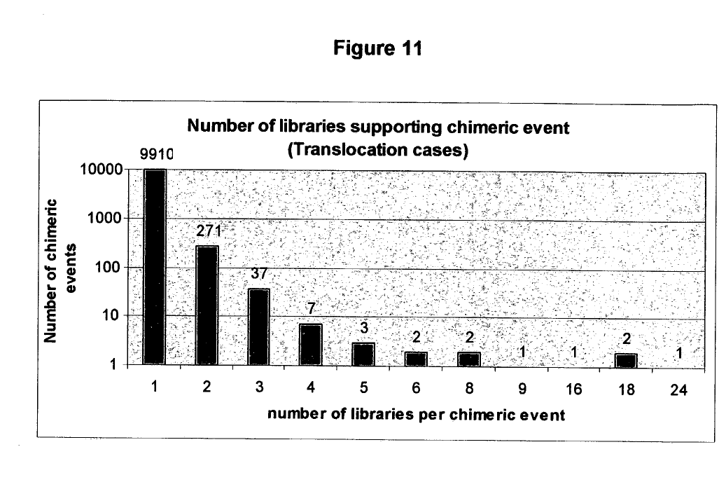

These results were further illustrated in a histogram shown in FIG. 10.

The distribution of the cDNA libraries supporting chimeric events which result from translocation is illustrated in Table 5, below, wherein the column “#Libraries” denotes the number of cDNA libraries which support a chimeric event resulting from translocation and the column “#Events” denotes the number of chimeric events. Note that in the preparation of the data every sequence which was annotated as mRNA in Genebank was considered as originating from a different library. In addition, un-annotated sequences which did not include library origin were not considered.

TABLE 5

| # Libraries | # Events |
|---|---|
| 9910 | 1 |
| 271 | 2 |
| 37 | 3 |
| 7 | 4 |
| 3 | 5 |
| 2 | 6 |
| 2 | 8 |
| 1 | 9 |
| 1 | 16 |
| 2 | 18 |
| 1 | 24 |

These results were further illustrated in a histogram shown in FIG. 11.

The distribution of the cDNA libraries which support deletion chimeric events is illustrated in Table 6, below, wherein the column “#Libraries” denotes the number of cDNA libraries which support a chimeric event resulting from deletion and the column “#Events” denotes the number of chimeric events.

TABLE 6

| # Libraries | # Events |
|---|---|
| 906 | 1 |
| 74 | 2 |
| 19 | 3 |
| 16 | 4 |
| 5 | 5 |
| 10 | 6 |
| 3 | 7 |
| 1 | 8 |
| 3 | 9 |
| 4 | 10 |
| 1 | 12 |
| 2 | 13 |
| 3 | 14 |
| 1 | 18 |

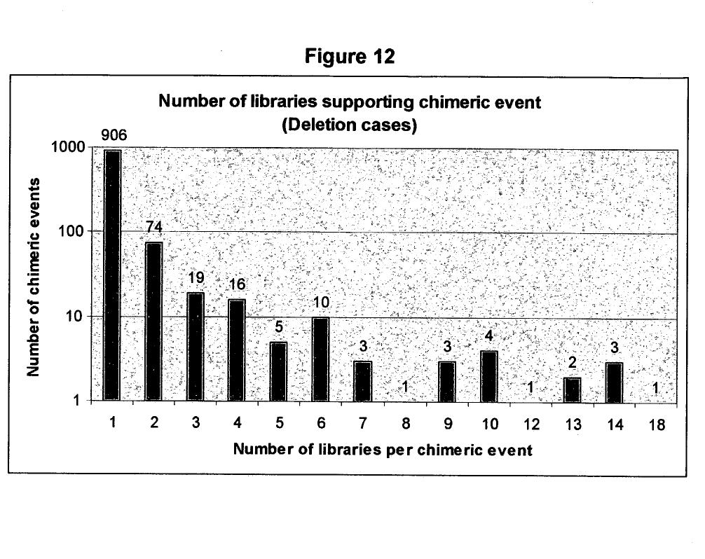

These results were further illustrated in a histogram shown in FIG. 12. The disribution of fusion events with several break points (i.e., Degree Type 1)—The distribution of chimeric events supporting the fusion between 2 genes (total cases) is illustrated in Table 7, below, wherein the column “# Events” denotes the number of chimeric events and the column “# Breaking points” denotes the number of chimeric events detected between the same two contigs participating in the chimeric event. It will be appreciated that in this case each event points to a different breaking point. The analysis excludes chimeric events including “contig-no contig” and “no contig-no contig” fusions.

TABLE 7

| # Events | # Breaking points |
|---|---|
| 6084 | 1 |
| 38 | 2 |
| 5 | 3 |
| 2 | 4 |
| 1 | 6 |

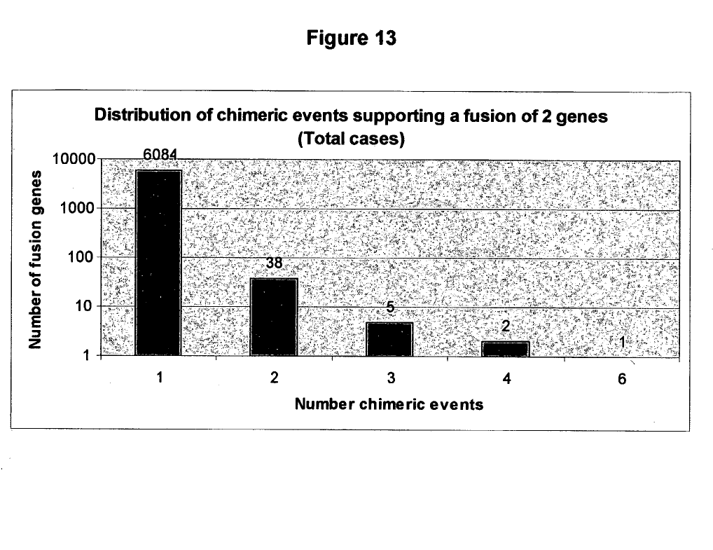

These results were further graphically analyzed as shown in FIG. 13.

The distribution of chimeric events supporting the fusion between 2 genes as a result of translocation is illustrated in Table 8, below.

TABLE 8

| # Events | # Breaking points |
|---|---|
| 5619 | 1 |
| 24 | 2 |
| 3 | 3 |
| 1 | 4 |
| 1 | 6 |

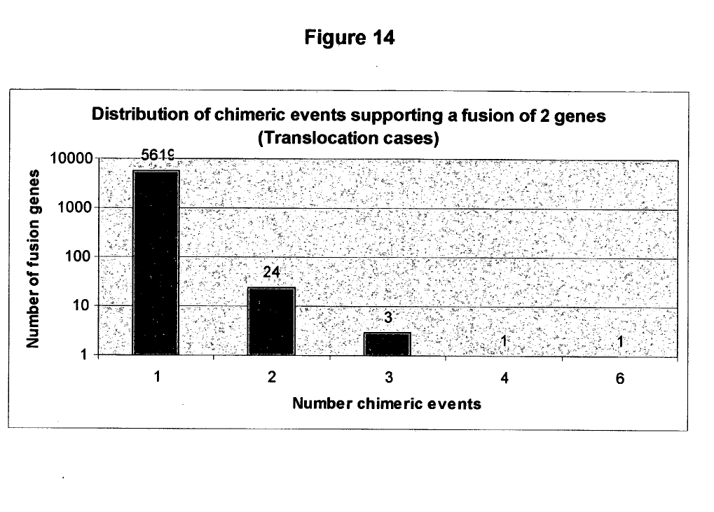

These results were further graphically analyzed as shown in FIG. 14.

The distribution of chimeric events supporting the fusion between 2 genes resulting from deletion is illustrated in Table 9, below.

TABLE 9

| # Events | # Breaking points |
|---|---|
| 465 | 1 |
| 14 | 2 |
| 2 | 3 |
| 1 | 4 |

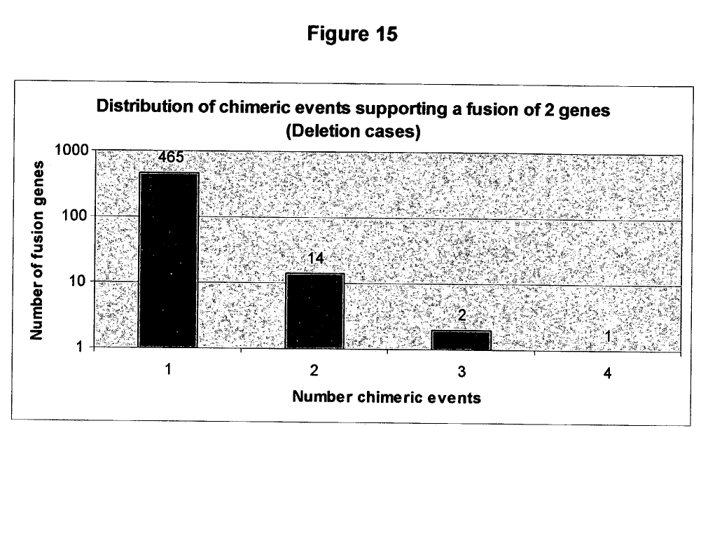

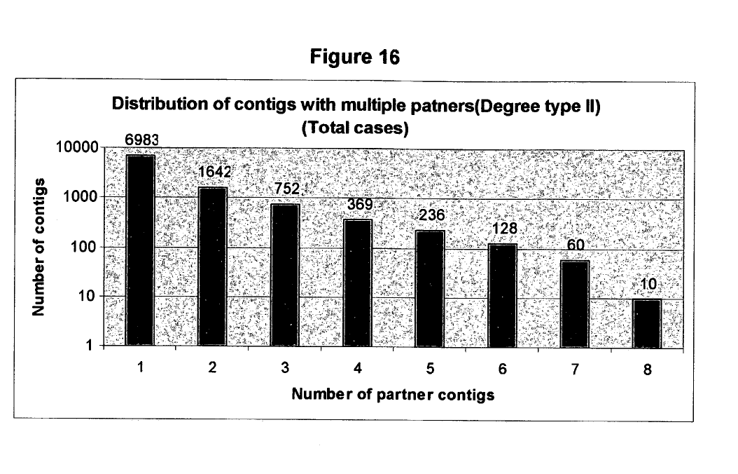

These results were graphically analyzed as shown in FIG. 15.

The biological basis for the breaking point analysis is that in some known translocation such as BCR-ABL, the breaking point between the 2 chromosome does not occoure on the same spot in all patients but rather in the same regions of the genes.

Distribution of contigs with multiple partners (i.e., degree type II)—The distribution of chimeric events occurring between a contig and several partner, (total events) is illustrated in Table 10, below, wherein the column “# contigs” denotes the number of contigs and the column “# partners” denotes the number of partners participating with the same contig in different chimeric events. It will be appreciated that the analysis presented herein excludes the chimeric events with the “no contig-no contig” cases.

TABLE 10

| # contigs | # partners |
|---|---|
| 6983 | 1 |
| 1642 | 2 |
| 752 | 3 |
| 369 | 4 |
| 236 | 5 |
| 128 | 6 |
| 60 | 7 |
| 10 | 8 |

These results were further analyzed graphically as shown in FIG. 16.

The distribution of chimeric events resulting from translocation and occurring between a contig and several partners is illustrated in Table 11, below.

TABLE 11

| # contigs | # partners |
|---|---|
| 6544 | 1 |
| 1510 | 2 |
| 680 | 3 |
| 358 | 4 |
| 192 | 5 |
| 103 | 6 |
| 45 | 7 |
| 8 | 8 |

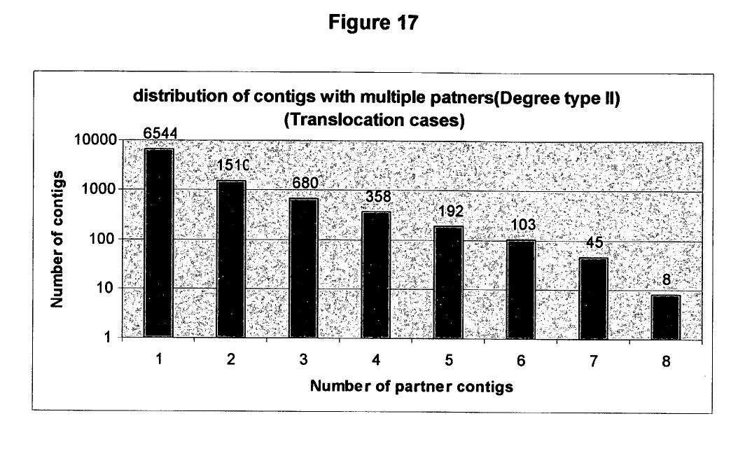

These results were further analyzed graphically as shown in FIG. 17.

The distribution of chimeric events resulting from deletion and occurring between a contig and several partners is illustrated in Table 12, below.

TABLE 12

| # contigs | # partners |
|---|---|
| 1148 | 1 |
| 117 | 2 |
| 12 | 3 |
| 9 | 4 |

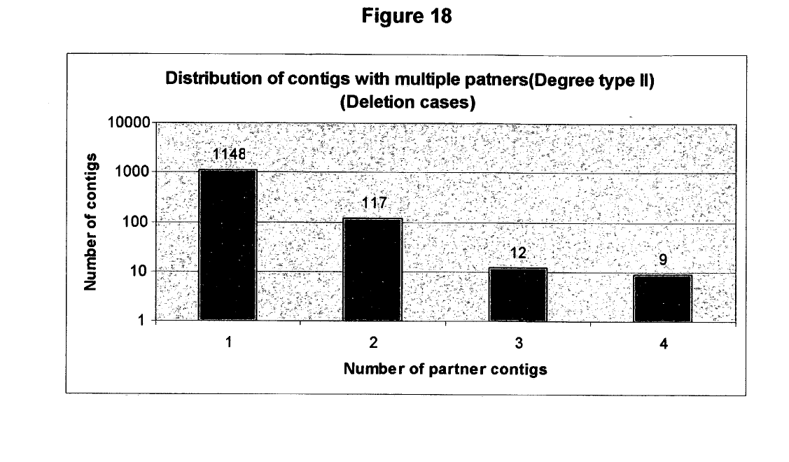

These results were further analyzed graphically as shown in FIG. 18.

Note, that this analysis (degree type II) was also used as a scoring parameter. Thus, the higher number of partner contigs found, the higher the pivot contig was scored, as described hereinabove and in examples 2-5, hereinbelow.

### Example 2

### “Hot Spot” Analysis Outline

Nucleic acid sequences at a transition point is tested for hotspot sequences. Hot spot sequences are particular areas of DNA, which are especially prone to spontaneous mutations or recombination. Defining a transition point sequences as hot spots supports the probability of a true fusion event.

Recurrence of chimeric events originating from the analysis of different tissue samples is indicative of a significant chimeric event Different degrees of significance were attributed to different analysis methods of fusion events (degree 0 to degree 3).

Degree-0—refers to the number of ESTs or the number of cDNA libraries supporting a single chimeric event.

Degree 1—refers to the number of chimeric events occurring between two contigs (gene)

Degree 2—refers to the number of chimeric events occurring between a certain contig and a set of different contigs.

Degree-3—refers to the number of chimeric events occurring in a certain genomic locus.

Degree-0—All cases in which more than one EST supported a chimeric event were assembled. The supporting ESTs were derived from different cDNA libraries, and matching the same breakpoint between gene A and gene B participating in the chimeric event, allowing precision of ten base pairs (i.e., transition points distant by ten base pairs are considered as a single transition point). While counting the libraries, consideration was given to their independent origin Libraries which were derived from the same parental tissue sample were considered as redundant and counted as one. This filter represents a biological phenomenon in which at different patients or types of tumors it is possible to identify identical fusion transcripts. For example, the translocations involve BCR-ABL fusions in CML, patients, PML-RARA in APL patients and many other recurrent translocations in sarcomas. (See examples in the file chimeric_contig_information.txt on the CD: CHIM011063, CHIM006763, CHIM007521 and CHIM008517).

Degree-1—All cases in which gene A and gene B created different fusion transcripts due to a fusion at different breakpoints or due to a creation of different alternative spliced products of the same chimeric event were assembled. For example this filter could identify unequal reciprocal events between gene A and gene B in the same library. The filter represents a biological process in which a known recurrent translocation is represented by various fusion products between the same two participating genes. For example in CML patients, researchers identified different products of the BCR-ABL fusion (p190, p200 and p210, see examples in the file chimeric_contig_information.txt on the attached CD-ROM. CHIM009985 and CHIM003503).

Degree-2—Several steps of analysis were effected in which in the first step, all chimeric events involving a certain gene (A) were grouped. In a second step, the number of different fusion partners was counted. For example, if gene A has independent chimeric events with genes B C D and E, it is defined as a donor for the creation of genomic rearrangements. In a third step, different characteristics of the fusion event were considered. For example, the genomic coordinates of the breakpoints were analyzed and checked for occurrence in the same region on the gene (see Examples 5a-d). Sequences that may promote genomic rearrangements were analyzed (see Example 6). The histological origin of the chimeric ESTs was further analyzed (i.e., tumorigenic origin versus normal origin). When chimeric sequences originated from tumor samples both the pathological and morphological characteristics were studied (see Examples 4a-c and 7). Changes in the regulation of gene expression and alterations in the function of the gene encoded protein that results from the rearrangement event were also looked for (see Example 15).

Degree-3—Identification of genomic loci which are lot spots for rearrangements. The distribution of the predicted breaking points on the chromosome was analyzed and regions on the chromosome which were rich in predicted breakpoints were identified. There is scientific evidence that some regions on the genome are predisposed to rearrangements (An evolutionary rearrangement of the Xp11.3-11.23 region in 3p21.3, a region frequently deleted in a variety of cancers [Timmer T, Terpstra P, van den Berg A, Veldhuis P M, Ter Elst A, van der Veen A Y, Kok K, Naylor S L, Buys CH.Genomics. 1999 Sep. 1;60(2):238-40]. Different “locus” definitions were used for different analysis:

(i) Assembled genomic contigs—are denoted as “NT” in Genbank annotations, wherein essentially a genomic contig is defined as a contiguous segment of the genome made by joining overlapping clones or sequences.

(ii) Accepted cytogentic bands such as 20q1, 20q2, 20p1 and the like.

(iii) Window size-using the “sliding windows approach (window of a fixed length [2 mega base pairs] sliding along the chromosome sequence), the number of breakpoints in the defined region is determined. A window that gives a significant pick is further analyzed. The results are compared to chromosomal aberrations and comparative genomic hybridization (CGH) databases. The statistic analysis also considered the quality of the genomic segment (NT) and the assembly gaps (see example 4).

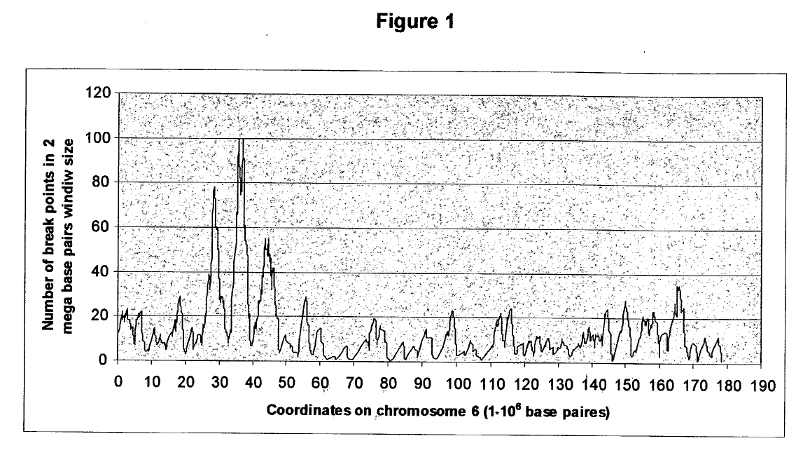

FIG. 1 shows that the region 25M-45M on chromosome 6 is rich in chromosomal rearrangement events.

### Example 3

### Identification of False Positive Fusion Transcripts Resultant of Library Construction

Identification of chimeric events generated during cDNA library construction includes in the first step a search for break points/EST-JUMPs in sequences relevant for library production like restriction enzymes recognition sequences, artificial adaptors, docking primer and stretches of adenosine or thymidine (A/T), which might represents the Oligo-dT used for first strand synthesis. The second step is to look for fusion transcripts that exhibit two different directional splicing, such that the canonical splice site GT-AG, GC-AG and AT-AC is identified on one sequence end and the opposite sequences CT-AC, CT-GC and GT-AT is identified on the other end. The third step is designated a “multi allelic event” and relates to the analysis of the Degree-2 level. It will be appreciated that when a certain gene that creates fusion transcripts with more than two different genes in a single library can be explained by the heterogeneity of the tumor. However it is more likely to be a false positive identification of a chimeric event.

### Examples 4a-c

### Degree Type2 Analysis

### Example 4a

### Degree Type 2 Analysis of the Chimeric Events which Involve the Prefoldin 5 (PFDN5) MM-1 Product of the Z40694 Contig

Prefoldin (GenBank Accession NM_{—002624}) is a hexameric molecular chaperone complex which includes two related classes of subunits, alpha and beta, and which is expressed in all eukaryotes and archaea. While eukaryotic prefoldin includes two related PFD-alpha subunits and four related PFD-beta sub units, bacterial prefoldin includes only one member of each class; a PFD-alpha subunit which closest human homolog is PFD5, and a PFD-beta subunit which closest human homolog is PFD6.

Prefoldin is known to interact with nascent polypeptide chains and can functionally substitute, in-vitro, for the Hsp70 chaperone system by stabilizing normative proteins for subsequent folding in the central cavity of a chaperonin. Molecular chaperones are proteins witch facilitate in the correct folding of other proteins in the crowded molecular environment that exists in living cells. Within this class of proteins, a key role is played by chaperonins, multisubunit toroidal assemblies that undergo major ATP-dependent conformational changes as part of the mechanism of facilitated folding.

The crystal structure of the prefoldin hexamer from the archaeum Methanobacterium thermoautotrophicum has been resolved lately by Siegert et al. [Siegert R, Leroux M R, Scheufler C, Hartl F U, Moarefi I. Structure of the molecular chaperone prefoldin: unique interaction of multiple coiled coil tentacles with unfolded proteins. Cell 2000 Nov. 10;103(4):621-32]. Interestingly, prefoldin has a jellyfish appearance, essentially including a body having a double beta-barrel assembly with six long tentacle-like coiled coils protruding from it. The distal regions of the coiled coils expose hydrophobic patches and are required for multivalent binding of normative proteins.

A yeast two-hybrid screen for c-myc using a HeLa cell cDNA library uncovered an interaction with MM1 cDNA [myc modulator-1, Mori K, Maeda Y, Kitaura H, Taira T, Iguchi-Ariga S M, Ariga H. MM-1, a novel c-Myc-associating protein that represses transcriptional activity of c-Myc. J Biol Chem 1998 Nov. 6;273(45):29794-800)]. The MM1 cDNA encodes a deduced 167-amino acid protein with a putative leucine zipper motif in the N terminus thereof. Northern blot analysis revealed expression of 4 distinct bands (0.7, 1.15, 2.9 and 4.4 kb); there was strong ubiquitous expression of the 0.7-kb transcript as well as strong expression of the 1.15-kb transcript in pancreas, weak expression in kidney, skeletal muscle, and placenta, and only very little expression in the liver and lung. Fluorescent microscopy showed MM1 expression primarily in the nucleus, with lower intensity in nucleoli and cytoplasm. Binding analyses indicated that MM1 and c-myc bind directly and that all but the N-terminal 13 amino acids of MM1 are required for binding. MM1 interacted with the myc box-2, a transcription-activating domain of c-myc. The MM1 protein was idependently purified biochemically as one of the subunit of the heterohexameric chaperone protein, designated prefoldin [Vainberg et al. Cell (1998) May 29;93(5):863-73]. In accord with the hereinbelow described results perfoldin is highly implicated in cancer and a deletion thereof from yeast resulted in impaired functions of the actin and tubulin-based cytoskeleton. These findings suggest that perfoldin is a non-redundant protein, which plays a central role in disease state such as cancer.

Chimeric events in which contig Z40694 is involved are described in Table 13, below. Essentially, each row identifies a predicted chimeric event between the contig and a partner contig:

Column 1—serial number of the row;

Column 2—identification of the chimeric event;

Column 3—contains characteristics of the fused sequence:

#AVG. EST JUMP—denotes the average of EST jump values eof the chimeric sequences which support the chimeric event.

#EST—denotes the accession number of the chimeric sequences which support the chimeric event.

#LIB—denotes the cDNA library of the chimeric sequences which support the chimeric event

#TUMOR/NORMAL/mix of tumor and normal/unknown—denotes the histology of the tissue used in the cDNA library construction.

#TISSUES LIST—denotes the origin of tissues used in the cDNA library construction.

Column 4—includes characteristics of the partner contig participating in the chimeric event including the name of the gene/locus and a short description of function if available;

Column 5—name of partner contig, degree type II value thereof (number of partners) and chimeric event identification

TABLE 13

| | PFDN5 chims | Chimeric sequences | Fusion partner | Partner deg |
|---|---|---|---|---|
| 1 | CHIM003053 | #AVG. EST JUMP: −6 | BTN2A1: butyrophilin, | F02772 (1) |
| | | #EST: BF899843 | subfamily 2, member A1 | CHIM003053 |
| | | #LIB: MT0223 | | |
| | | #TUMOR | | |
| | | chronic myclogenous | | |
| | | leukemia, flow-sorted | | |
| | | #TISSUES LIST: | | |
| | | whole blood | | |
| 2 | CHIM004958 | #AVG. EST JUMP: −1 | LOC254170: similar to F- | No contig |
| | | #EST: AA580941: | box and leucine-rich repeat | |
| | | #LIB: NCI_CGAP_GC1 | protein 3B; F-box protein | |
| | | #TUMOR | Fb13b | |
| | | seminoma | | |
| | | #TISSUES LIST: | | |
| | | Germ cell | | |
| 3 | CHIM010670 | #AVG. EST JUMP: 1 | CD47 antigen (Rh-related | HSOA3MR(4) |
| | | #EST: BF034675 | antigen, integrin-associated | CHIM010670 |
| | | #LIB: NIH_MGC_66 | signal transducer) | CHIM007506 |
| | | #TUMOR | | CHIM004268 |
| | | Ovary adenocarcinoma | | CHIM008978 |
| | | Cell line | | |
| | | #TISSUES LTST: | | |
| | | endocrine, ovary | | |
| 4 | CHIM011299 | #AVG. EST JUMP: −3 | DOC-1R | R31638 (4) |
| | | #EST: AI351791 | tumor suppressor deleted in | CHIM007210 |
| | | #LIB: NCI_CGAP_GC4 | oral cancer-related | CHIM011299 |
| | | #TUMOR | | CHIM000868 |
| | | #TISSUES LIST: | | CHIM004817 |
| | | Germ cell | | |

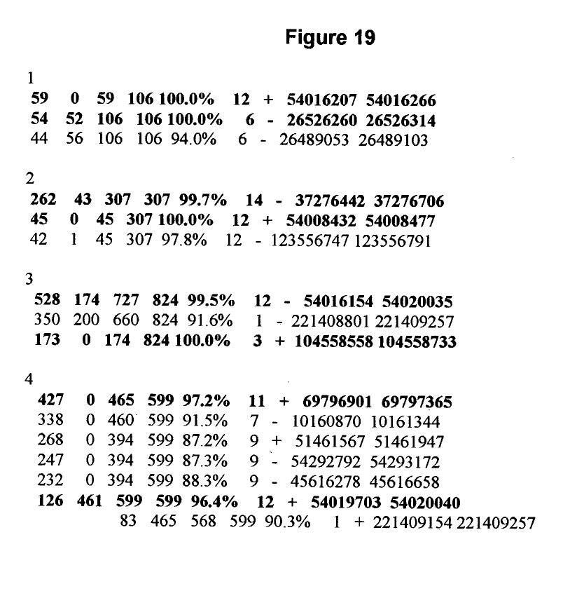

The alignments of the chimeric sequences to the genome are shown in FIG. 19.

Examples 4b-c describe chimeric events involving the Z40694 partner contigs.

### Example 4b

### Contig: R31638 Product: Growth Suppressor Related (DOC-1R)

Chimeric events involving R31638 identified according to the teachings of the present invention are illustrated in Table 14, below.

TABLE 14

| | DOC-1R chims | Chimeric sequences | Fusion partner | Partner deg |
|---|---|---|---|---|
| 1 | CHIM007210 | #AVG. EST JUMP: 37 | hypoxia-inducbile gene 1 (HIG1) | M78775 (5) |
| | | #EST: AV705308 | | CHIM007210 |
| | | #LIB: ADB | | CHIM002926 |
| | | #NORMAL | | CHIM003727 |
| | | #TISSUES LIST: | | CHIM005246 |
| | | adrenal | | CHIM001418 |
| 2 | CHIM011299 | #AVG. EST JUMP: −3 | prefoldin 5 (PFDN5) MM-I | Z40694 (4) |
| | | #EST: A1351791 | | CHIM011299 |
| | | #LIB: NCI_CGAP_GC4 | | CHIM010670 |
| | | #TUMOR | | CHIM004958 |
| | | #TISSUES LIST: | | CHIM003053 |
| | | Germ cell tumor | | |
| 3 | CHIM000868 | #AVG. EST JUMP: −4 | Hypothetical protein | T57492 (1) |
| | | #EST: AW264211: | DKFZp586M1819 | CHIM000868 |
| | | #LIB: NCI_CGAP_Bm53 | LOC253897: similar to putative | |
| | | #TUMOR | lysophosphatidic acid | |
| | | #TISSUES LIST: | acyltransferase | |
| | | brain, meningioma | | |
| 4 | CHIM004817 | #AVG. EST JUMP: −6 | PRO1777: hypothetical protein | T06332 (4) |
| | | #EST: AU144543 | PRO1777 | CHIM006627 |
| | | #LIB: HEMBA1 | or | CHIM000001 |
| | | #NORMAL | T-cell lymphoma tumor antigen | CHIM003686 |
| | | TISSUES LIST: | se70-2 (SE70-2) | CHIM004817 |
| | | embryo, head | | |

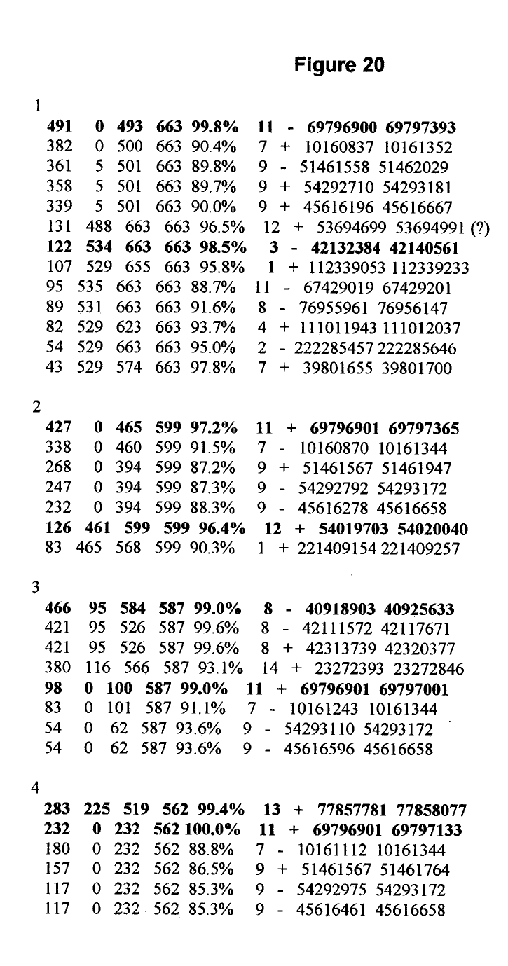

The alignments of the chimeric sequences to the genome are shown in FIG. 20. Note that the R31638 gene product is implicated in cancer. Specifically, DOC-1R has been suggested to be a tumor suppressor gene [Zhang Biochem Biophys Res Commun (1999) February 5;255(1):59-63]. Thus, a translocation involving same may lead to tumor initiation and cancer progression.

### Example 4c

### Contig: HSOA3MR Product: CD47 Antigen (Rh-Related Antigen, Integrin-Associated Signal Transducer)

Chimeric events involving HSOA3MR identified according to the teachings of the present invention are illustrated in Table 15, below.

TABLE 15

| | HSOA3MR chims | Chimeric sequences | Fusion partner | Partner deg |
|---|---|---|---|---|
| 1 | CHIM010670 | #AVG. EST JUMP: 1 | prefoldin 5 (PFDN5) | Z40694 (4) |
| | | #EST: BF034675: | MM-1 | CHIM011299 |
| | | #LIB: NIH_MGC 66 | | CHIM010670 |
| | | #TUMOR | | CHIM004958 |
| | | adenocarcinoma | | CHIM003053 |
| | | #TISSUES LIST: | | |
| | | Ovary, Cell line | | |
| 2 | CHIM007506 | #AVG. EST JUMP: −3 | insulin-like growth | S56205 (6) |
| | | #EST: AI167998 | factor binding protein | CHIM003333 |
| | | #LIB: Soares_senescent | 3 (IGFBP3) | CHIM000041 |
| | | fibroblasts_NbHSF | | CHIM000077 |
| | | #NORMAL | | CHIM007506 |
| | | #TISSUES LIST: | | CHIM008552 |
| | | | | CHIM007081 |
| 3 | CHIM004268 | #AVG. EST JUMP: −18 | TBC1D1: TBC1 (tre- | Z38201 (5) |
| | | #EST: BF996478 | 2/USP6, BUB2, | CHIM002815 |
| | | #LIB: GN0123 | cdc16) domain | CHIM008342 |
| | | #NORMAL | family, member 1 | CHIM007742 |
| | | #TISSUES LIST: | | CHIM000943 |
| | | placenta | | CHIM005854 |
| | | | | (here no-con) |
| 4 | CHIM008978 | #AVG. EST JUMP: 91 | SERPINA3: serine | HUMA1ACM |
| | | #EST: AW819120 | (or cysteine) | (4) |
| | | #LIB: ST0281 | proteinase inhibitor, | CHIM008050 |
| | | #TUMOR | clade A | CHIM008978 |
| | | carcinoma | (alpha-1 | CHIM101443 |
| | | #TISSUES LIST: | antiproteinase, | CHIM010487 |
| | | Stomach | antitrypsin), member 3 | |

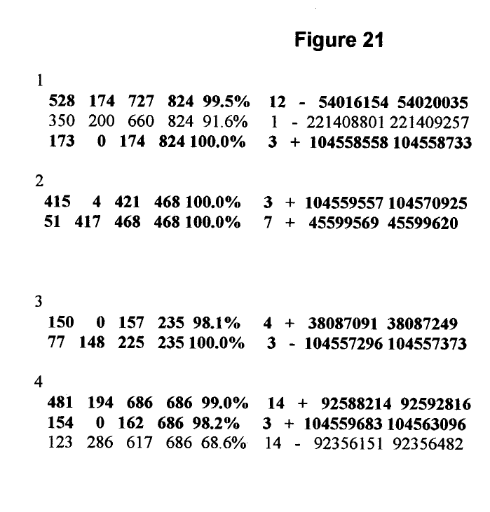

The alignments of the chimeric sequences to the genome are shown in FIG. 21.

CD47 or Integrin-associated protein (IAP) (Gene Bank Accession No. NM_{—001777}) is a 50-kD membrane protein with an amino-terminal immunoglobulin domain and a carboxyl-terminal multiple-membrane-spanning region. CD47 is involved in the increase of intracellular calcium concentration which occurs upon cell adhesion to extracellular matrix IAP/CD47 is was also found to be identical to a previously isolated OA3, an ovarian carcinoma antigen (Mawby et al. Am. J. Hum. Genet 41: 1061-1070, 1987). Interestingly, IAP protein is also expressed in crythrocytes, which have no known integrins.

Lindberg et al. (J. Biol. Chem. 269: 1567-1570, 1994) showed that IAP expression is reduced in Rh(null) crythrocytes. Fluorescence in situ hybridization studies showed that the IAP structural gene maps to 3q13.1-q13.2, within a region known to contain a gene encoding the Rh-associated 1D8 antigen. By expression studies on human erythrocytes and IAP transfectants, IAP was shown to be identical to the 1D8 antigen and to CD47, a cell surface protein with broad tissue distribution reduced in expression on Rh(null) erythrocytes. Lindberg et al. stated that these studies demonstrated an unexpected link between integrin signal transduction and erythrocyte membrane structure.

A host defence function was attributed to IAP usinf genetic manipulation studies in mice [Lindberg (1994) J. Biol. Chem. 269:1567-1570]. IAP may participate in polymorphonuclear migration in response to bacterial infection and in polymorphonuclear activation at extravascular sites. Mice homozygous for knockout of the IAP gene succumbed to Escherichia coli peritonitis at inoccula survived by heterozygous littermates. In vivo, such mice exhibited an early defect in PMN accumulation at the site of infection. Furthermore, such micse showed deficiency of several manifestations of PMN activation.

The immune system recognizes invaders as foreign because they express determinants that are absent on host cells or because they lack ‘markers of self’ that are normally present Oldenborg et al. (Science 288: 2051-2054, 2000) demonstrated that CD47 functions as ‘a marker of self’ in murine red blood cells. Red blood cells that lack CD47 were rapidly cleared from the bloodstream by splenic red pulp macrophages. CD47 on normal red blood cells prevented this elimination by binding to the inhibitory receptor signal regulatory protein alpha (SIRP-alpha). Thus, Oldenborg et al. concluded that macrophages may use a number of nonspecific activating receptors and rely on the presence or absence of CD47 to distinguish self from foreign. Oldenborg et al. suggested that CD47-SIRP-alpha may represent a potential pathway for the control of hemolytic anemia. Osteoclasts and giant cells are multinucleated and resorb the substrate onto which they adhere They are thought to originate from the fusion of mononuclear phagocytes. Han et al. (J. Biol. Chem. 275: 37984-37992, 2000) used immunofluorescence microscopy to show that at the onset of fusion macrophages express not only the macrophage fusion receptor (MFR, or SIRP-alpha) but also, at a lower level than MFR, the hemopoietic form of CD47. Immunoprecipitation and immunoblot experiments confirmed the association of the CD47 variable domain and the MFR immunoglobulin V1 domain. Macrophage fusion could be blocked by either anti-CD47 monoclonal antibodies or a CD47 fusion protein.

Altogether contig Z40694 is highly scored since all its chimeric sequences originate from tumor tissues and participate in cancer and disease development and progression.

### Examples 5a-d

### Degree Type2 Analysis

### Contig: D12334 Product: Clathrin Heavy Chain Gene, CLTC

Clathrin is the major protein constituent of the coat that surrounds the cytoplasmic face of the organelles (coated vesicles) mediating selective protein transport [Goldstein Nature (1979) 279:679-685]. Clathrin coats are involved in receptor-mediated endocytosis, localization of resident membrane proteins to the trans-Golgi network, and transport of proteins to the lysosome/vacuole (Schmid Annu Rev Biochem (1997) 66.511-5481. Recently, clathrin has also been immunodetected in the mitotic spindle, suggesting a novel role for clathrin in mitosis or a novel regulatory mechanism for localization of clathrin in mitotic cells [Okamoto Am. J. Physiol. (2000) 279:C369-C374].

Clathrin is a three-legged molecule, termed a triskelion, composed of heavy and light chains. Two clathrin heavy chain genes exist in the genome, clathrin heavy chain gene (CLTC), which has been localized to 171q23 [Dodge Genomics (1991) 11:174-179], and clathrin heavy chain polypeptide-like gene (CLTCL) that has been localized to 22q11.2 [Kedra Hum Mol Genet (1996) 5:625-631]. Fusion transcripts involving CLTC have been previously reported [Bridge J A, Kanamori M, Ma Z, Pickering D, Hill D A. Lydiatt W, Lui M Y, Colleoni G W, Antonescu C R, Ladanyi M. Morris S W. Fusion of the ALK gene to the clathrin heavy chain gene, CLTC, in inflammatory myofibroblastic tumor. Am J Pathol. 2001 August;159(2):411-5; Cools J, Wlodarska I, Somers R, Mentens N., Pedeutour F, Maes B, De Wolf-Peeters C, Pauwels P, Hagemeijer A, Marynen P. Identification of novel fusion partners of ALK, the anaplastic lymphoma kinase, in anaplastic large-cell lymphoma and inflammatory myofibroblastic tumor. Genes Chromosomes Cancer 2002 August 34:354-62].

In many chimerism events inclolving CLTC, the fusion point in the CLTC transcript is close to the 3′ end thereof, thus conserving nearly all of the clathrin heavy chain, including the motifs responsible for triskelion assembly. However, the clathrin moiety in the CLC-ALK fusion probably promotes constitutive activation and relocalization of the ALK kinase domain from its normal position at the inner surface of the cell membrane in neural cells to the cytoplasm of myofibroblastic cells. This translocation [i.e., t(2;17)(p23;q23)] typically promotes inflammatory myofibroblastic tumors.

Other chimeric events which involve the D12334 contigs were identified according to the teachings of the present invention and are illustrated in Table 16, below. The table format is described in Example 4a, hereinabove.

TABLE 16

| | CTLC chims | Chimeric sequences | Fusion partner | Partner degree |
|---|---|---|---|---|
| 1 | CHIM007742 | #AVG. EST JUMP: −6 | The gene encoding TBC1D1 with | Z38201 (5) |
| | | #EST: AW014054 | homology to the tre-2/USP6 | CHIM002815 |
| | | #LIB: NCI_CGAP_Sub1 | oncogene, BUB2, and cde16 maps | CHIM008342 |
| | | mix of tumor and normal | to mouse chromosome 5 and | CHIM007742 |
| | | #TISSUES LIST: | human chromosome 4. | CHIM000943 |
| | | mix of tissues | | CHIM005854 |
| 2 | CHIM004687 | #AVG. EST JUMP: −4 | H2A histone family, member Y | T05149 (4) |
| | | #EST: BF7063101 | (H2AFY) | CHIM010585 |
| | | #LIB: NCI_CGAP_Co16 | | CHIM010996 |
| | | #TUMOR | | CHIM005127 |
| | | #TISSUES LIST: | | CHIM004687 |
| | | Colon | | |
| 3 | CHIM001703 | #AVG. EST JUMP: −4 | LOC202325: similar to hypothetical | R27953 (4) |
| | | #EST: AI989296 | protein DKFZp761D221 | CHIM002958 |
| | | #LIB: prostate cancer cell line | | CHIM004504 |
| | | LNCaP | | CHIM002339 |
| | | #TUMOR | | CHIM001703 |
| | | #TISSUES LIST: | | |
| | | prostate | | |
| 4 | CHIM010226 | #AVG. EST JUMP: 11 | KIAA1962 protein similar to zinc | AF026101 (1) |
| | | #EST BE173551: | finger protein HIT-10 | CHIM010226 |
| | | #LIB: HT0560 | Or | |
| | | #TUMOR | FLI30791 (FLI30791), mRNA. f- | |
| | | #TISSUES LIST: | C2H2: Region: Zinc finger. C2H2 | |
| | | head & neck thyroid | type | |
| | | | * All multi hits are associated | |
| | | | with zink fingers and krab | |
| | | | domains | |
| 5 | CHIM003213 | #AVG. EST JUMP: 23 | ribosomal protein L17 (RPL17) | HSL23MR (2) |
| | | EST: AW957886, AW957809 | | CHIM008037 |
| | | #LIB: MAGE resequences, MAGE | | CHIM003213 |
| | | Histology, Tissue: unknown | | |
| 6 | CHIM002601 | #AVG. EST JUMP: −8 | Near DDX11 | No contig but hits |
| | | #EST: BF762825: | | Near: NM030655 |
| | | #LIB: CS0032 | | R94261 (2) |
| | | #TUMOR | | CHIM008037 |
| | | adenocarcinoma, cell line | | CHIM001545 |
| | | #TISSUES LIST: | | |
| | | Colon | | |

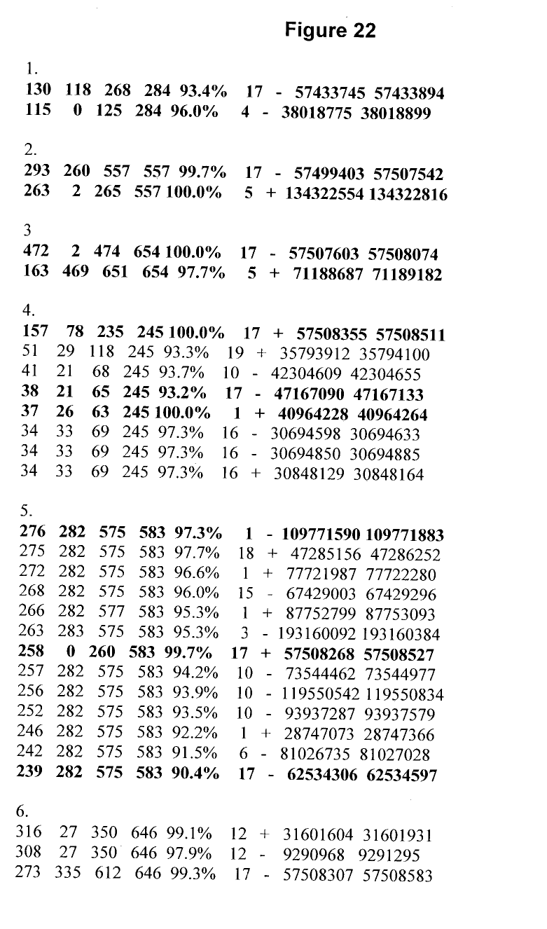

The alignments of the chimeric sequences to the genome is shown in FIG. 22.

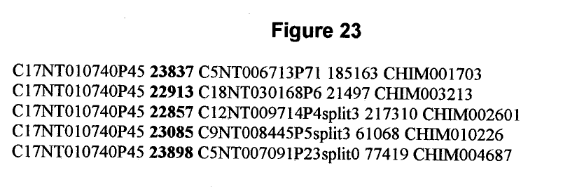

The breakpoints of the chimeric events on the genome are shown in FIG. 23.

All of the breakpoints are clustered to 1 Kb in the last exon, which participates in the known translocation of ALK-CLTC t(2;17)(p23;q23), described hereinabove.

Examples 5b-d describe chimeric events involving D12334 partner contigs.

### Example 5b

### Contig:Z39201 Product: TBC1D1 (XM-035618)

A description of the chimeric events in which the contigs involve is described in Table 17, below.

TABLE 17

| | Z38201 chims | Chimeric ESTs | Fusion partner | Partner degree |
|---|---|---|---|---|
| 1 | CHIM002815 | #AVG. EST JUMP: 1 | On 14 2 hits | HSIGM1M2 (1) |
| | | #EST: AI469164 | 30 kb from each other | CHIM002815 |
| | | #LIB: NCI_CGAP_Lym5 | tandem repeat | |
| | | #TUMOR | no gene | |
| | | #TISSUES LIST: | | |
| | | lymph node, follicular lymphoma | | |
| 2 | CHIM008342 | #AVG EST JUMP: −13 | DKFZP56611024 | T74386 (1) |
| | | #EST: BF771965 | In genecarta | CHIM008342 |
| | | #LIB: IT0023 | connected to | |
| | | #TUMOR | PHKG1: | |
| | | #TISSUES LIST: | phosphorylase kinase, | |
| | | carcinoma, epididymis | gamma 1 | |
| 3 | CHIM007742 | #AVG. EST JUMP: −6 | CLTC | D12334 (6) |
| | | #EST: AW014054 | | See above |
| | | #LIB: NCI_CGAP_Sub1 | | Table D12334 |
| | | #mix of Tumor + normal | | |
| | | #TISSUES LIST: | | |
| | | mix of tissues | | |
| 4 | CHIM000943 | #AVG. EST JUMP: 2 | LOC223093: similar | No contig |
| | | #EST: BF996030: | to dJ475N16.3 (novel | |
| | | #LIB: GN0173 | protein similar | |
| | | #NORMAL | toRPL7A (60S | |
| | | #TISSUES LIST: | ribosomal protein | |
| | | placenta | L7A)) | |
| 5 | CHIM005854 | #AVG. EST JUMP: −4 | LRRN1: leucine-rich | AA078321 (1) |
| | | #EST: BE709716 | repeat protein, | CHIM005854 |
| | | #LIB: HT0618 | neuronal 1 | |
| | | #TUMOR | | |
| | | #TISSUES LIST: | | |
| | | head & neck | | |
| | | thyroid, carcinoma | | |

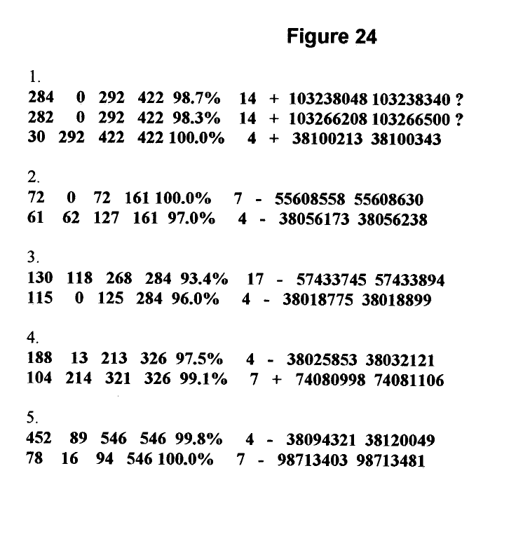

The alignments of the chimeric sequences to the genome is shown in FIG. 24.

### Example 5c

### Contig: T05149 Product: H2M Histone Family, Member Y, Isoform 2 (GenBank Accession No. NM-004893)

Histones are basic nuclear proteins which are responsible for the nucleosome structure of the chromosomal fiber in eukaryotes. Nucleosomes consist of approximately 146 bp of DNA wrapped around a histone polypeptide octamer composed of pairs of each of the four core histones (H2A, H2B, H3, and H4). The chromatin fiber is further compacted through the interaction of a linker histone, H1, with the DNA inbetween the nucleosomes to form higher order chromatin structures.

A description of the chimeric events in which the T05149 contig is involved is provided in Table 18, below.

TABLE 18

| | T05149 chims | Chimeric ESTs | Fusion partner | Partner deg |
|---|---|---|---|---|
| 1 | CHIM010585 | #AVG EST JUMP: 24 | hypothetical protein | F13665 (3) |
| | | #EST: BG615404 | FLJ20073 | CHIM010585 |
| | | #LIB: NIH_MGC_61 | | CHIM005408 |
| | | #TUMOR | | CHIM005577 |
| | | #TISSUES LIST | | |
| | | testis, embryonal carcinoma, cell | | |
| | | line | | |
| 2 | CHIM010996 | #AVG. EST JUMP: −4 | Homo sapiens RACK- | M85494 (3) |
| | | #EST: AI630327 | like protein PRKCBPI | CHIM010996 |
| | | #LIB: Proliferating Erythroid Cells | (Human protein kinase | CHIM007905 |
| | | (LCB: ad library) | C-binding protein | CHIM002350 |
| | | #NORMAL | RACK7) | |
| | | #TISSUES LIST | | |
| | | whole blood | | |
| 3 | CHIM005127 | #AVG. EST JUMP: −5 | Hits both strands; | Z42077 (3) |
| | | #EST: AW205111 | Homo sapiens | CHIM004386 |
| | | #LIB: NCI_CGAP_Sub3 | apoptosis regulator | CHIM005127 |
| | | Mix normal/tumor | BCI-G (BCLG), | CHIM008856 |
| | | #TISSUES LIST | transcript variant 3 (hits | |
| | | mixed tussues | an intron) | |
| | | | low density lipoprotein | |
| | | | receptor-related protein | |
| | | | 6 (hits an exon) | |
| 4 | CHIM004687 | #AVG. EST JUMP: −4 | CLTC | D12334 (6) |
| | | #EST BF063101 | | See above |
| | | #LIB: NCI_CGAP_Co16 | | Table D12334 |
| | | #TUMOR | | |
| | | #TISSUES LIST | | |
| | | colon | | |

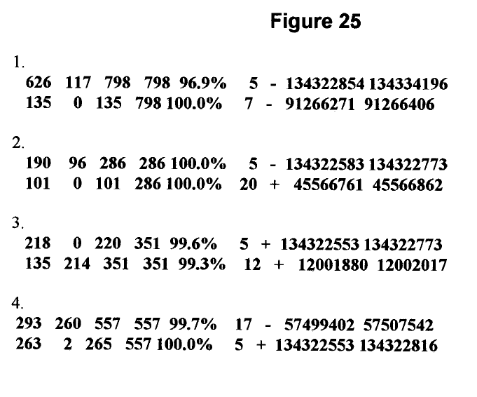

The alignments of the chimeric sequences to the genome is shown in FIG. 25.

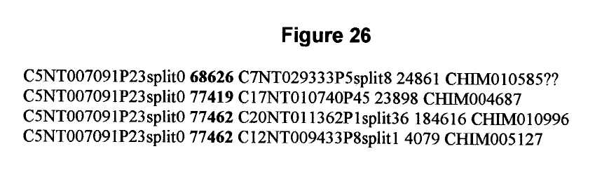

The distribution of breakpoints of the chimeric events on the genomic sequence are shown in FIG. 26. Note that three of the breakpoints are clustered to a 300 base pair range. The H2A histone family is known to be involved in cancer.

### Example 5d

### Contig: R27953 Product: LOC202325. Similar to Hypothetical Protein DKFZp761D221

Characteristics of the chimeric events in which R27953 is involved are provided in Table 19, below.

TABLE 19

| | R27953 chims | Chimeric ESTs | Fusion partner | Partner deg |
|---|---|---|---|---|
| 1 | CHIM002958 | #AVG. EST JUMP: −4 | — | No contig |
| | | #EST: BE005026 | | |
| | | #LIB: BN0115 | | |
| | | #NORMAL | | |
| | | #TISSUES LIST: | | |
| | | mammary gland, cell line | | |
| 2 | CHIM004504 | #EVG. EST JUMP: −15 | GART — | No contig |
| | | #EST BF763230 | phosphoribosylglycinamide | |
| | | #LIB CS0049 | formyltransferase (intron) | |
| | | #TUMOR | | |
| | | #TISSUES LIST: | | |
| | | colon, cell line | | |
| 3 | CHIM002339 | #AVG. EST JUMP: −3 | ZNF271 (zinc finger protein | AA383181 (3) |
| | | #RNA: AF153201 | 271) | CHIM002339 |
| | | #NORMAL | | CHIM006326 |
| | | #TISSUES LIST: | | CHIM002491 |
| | | hair dermal papilla | | |

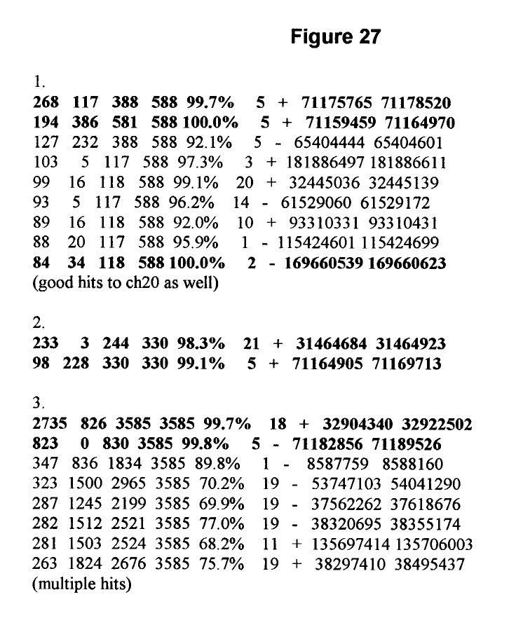

The alignments of the chimeric sequences to the genome are shown in FIG. 27.

### Example 6

### Degree Type 3 Analysis and Identification of “Hot Spots”

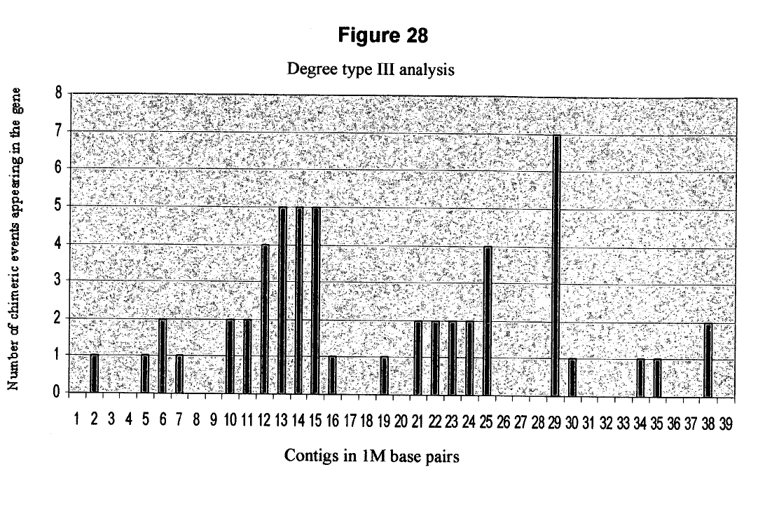

1 Mb region on chromosome 3 with multiple hits of chimeric sequences was analyzed (FIG. 28). The region is predicted to be involved in recurrent chromosomal rearrangements. From the list of contigs characterized as degree type 3 essentially implicated in multiple rearrangements, as shown in FIG. 28, gene #29 was further degree type 2 analysed:

Degree Type 2 Analysis for Contig: T10051 (i.e., genE 29) Product: RNA Binding Motif Protein 5 (RBM5)

Characteristics of the chimeric events in which T10051 is involved are illustrated iii Table 20, below.

TABLE 20

| | T10051 chims | Chimeric ests | Fusion partner | Partner deg |
|---|---|---|---|---|
| 1 | CHIM010537 | #AVG. EST JUMP: 14 | HSA250839: NM_018401 | No contig |
| | | #EST: AW818419 | gene for serine/threonine protein | The chimeric |
| | | #LIB: ST0278 | kinase | sequence hits |
| | | #TUMOR | And HNRPD: NM_002138 | near contig |
| | | carcinoma | heterogeneous nuclear | R55975 (1) |
| | | #TISSUES LIST; | ribonucleoprotein D (AU-rich | CHIM010537 |
| | | stomach | element RNA binding protein 1, | |
| | | | 37 kDa) | |
| 2 | CHIM000099 | #AVG. EST JUMP: 35 | Genomic region | AW373727 (1) |
| | | #EST: AW373727 #LIB: | The proximate gene is | CHIM000099 |
| | | BT0536 | osteoclast stimulating factor 1 on | |
| | | #TUMOR | ch9 | |
| | | carcinoma, cell line | | |
| | | #TISSUES LIST: mammary gland | | |
| 3 | CHIM001078 | #AVG. EST JUMP: −1 | Genomic region near | R07313 (1) |
| | | #EST: AL555315 | LOCI23722: similar to stretch | CHIM001078 |
| | | #LIB: LTI_NFL006_PL2 | response protein 553 | |
| | | #NORMAL | | |
| | | #TISSUES LIST: Placenta | | |
| 4 | CHIM002024 | #AVG. EST JUMP: 0 | LOC221632: hypothetical gene | No contig |
| | | #EST: AU118664 | supported by AK026189; | |
| | | #LIB: HEMBAI | AK026189 AK026189; | |
| | | #NORMAL | AK022865; AK026189 | |
| | | #TISSUES LIST: | | |
| | | embryo, head | | |
| 5 | CHIM009616 | #AVG. EST JUMP: 9 | Genomic region | No contig |
| | | #EST: AW858870 | Near ERAL1: Era G-protein-like 1 | |
| | | #LIB: CT0347 | cell cycle protein | |
| | | #TUMOR | | |
| | | carcinoma | | |
| | | #TISSUES LIST: | | |
| | | colon | | |
| 6 | CHIM006473 | #AVG. EST JUMP: 5 | hypothetical protein FLJ21080 | AW799619 (1) |
| | | #EST: BE074793 | inciude SET domains protein- | CHIM006473 |
| | | #LIB: BT0578 | protein interaction domains | |
| | | #TUMOR | | |
| | | carcinoma, cell line | | |
| | | #TISSUES LIST: | | |
| | | mammary gland | | |
| 7 | CHIM003162 | #AVG. EST JUMP; 4 | DAPK1 | No contig |
| | | #EST: B1049651 | death-associated protein kinase 1 | |
| | | #LIB: GN0288 | It is a tumor suppressor candidate. | |
| | | #NORMAL (?) | | |
| | | #TISSUES LIST: unknown | | |
| | | (placenta?) | | |

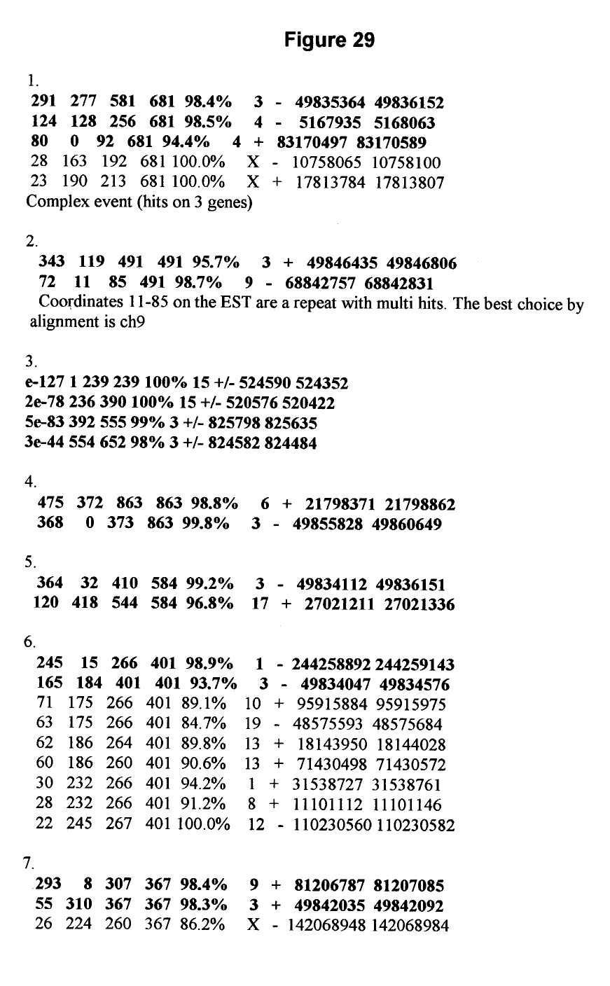

The alignments of the chimeric sequences to the genome are shown in FIG. 29.

Sequence repeat analysis:

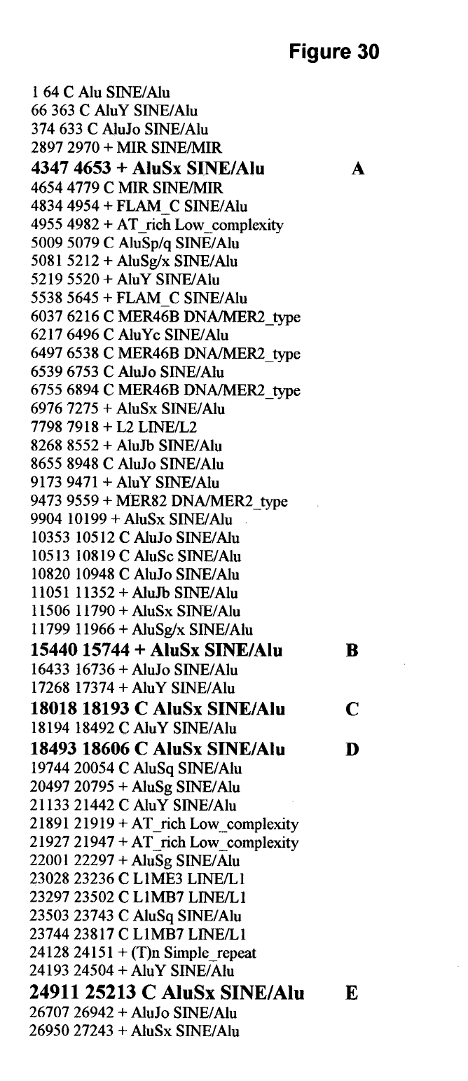

Although the transition point in the RBM5 (GenBank Accession No: NM_{—}005778) are partially clustered and partially dispersed at different regions of the gene a correlation between low complexity (e.g., repetitive) sequence elements and occurance of predicted breaking points was addressed (see FIG. 30).

Chimeric events 1,5 and 6 are clustered to ˜1 KB region as shown in FIG. 30 wherein the corresponding transition points were allocated to 24911-25213 coordinates on gene 29 (i.e., E).

Chimeric events 2,3 and 7 are clustered to 3 Kb region, as shown in FIG. 30 B-D.

All the chimeric events were found in the vicinity of AluSx see repeats (A=4, B=2, C=3, D=7 and E=1,5 and 6 numbers represent the chimeric event in Table 20) and some were found in the fusion partner. These observations suggest a general mechanism for genomic rearangments and may be used as a method to validate fusion transcripts.

RBM5 expression—Note that the RBM5 gene is implicated in carcinogenesis as suggested by the observation that a chromosomal segment corresponding to 3p21.3 is deleted in the small cell lung cancer cell line GLC20. This segment was found to include the RBM5 gene encoding a deduced 815-amino acid protein that contains two RNA-binding motifs, a C2C2-type zinc finger motif a C₂H₂-type zinc finger motif, and a bipartite nuclear signal. The same functional motifs were also found in RBM6 (GenBank accession No: NM-005777)[Gure A O, Altorki N K, Stockert E, Scanlan M J, Old L J, Chen Y T. Human lung cancer antigens recognized by autologous antibodies: definition of a novel cDNA derived from the tumor suppressor gene locus on chromosome 3p21.3. Cancer Res 1998 Mar. 1;58(5):1034-41; Timmer et al. An evolutionary rearrangement of the Xp11.3-11.23 region in 3p21.3, a region frequently deleted in a variety of cancers. Genomics 1999 Sep. 1;60(2):238-40].

It will be appreciated that both RBM5 and RBM6 share 30% amino acid identity. Northern blot analysis of multiple tissues detected expression of both transcripts in all tissues investigated. However, although the RBM5 gene is located in a region considered critical for the development of lung cancer, Northern and Southern analyses of a number of lung cancer cell lines revealed no aberrant expression (i.e., deletions, insertions, translocations) of the RBM5 transcript.

Lerman et al. showed involvement of the RBM5 gene region in chromosomal aberrations relating to cancer [Lerman The 630-kb lung cancer homozygous deletion region on human chromosome 3p21.3: identification and evaluation of the resident candidate tumor suppressor genes. The International Lung Cancer Chromosome 3p21.3 Tumor Suppressor Gene Consortium Cancer Res 2000 Nov. 1;60(21):6116-33]. According to Lerman, the region of chromosome 3p21.3 is associated with a putative lung cancer tumor suppressor gene. The mouse ortholog is 97% identical on the protein level. Northern blot analysis detected wide expression in normal and lung cancer tissues and cell lines.

RBM5 function—Drabkin et al. have found that recombinant proteins containing the RNA recognition motifs of RBM5 or RBM6 specifically bind poly(G) RNA homopolymers in vitro [Drabkin Oncogene (1999) April 22;18(16):2589-97).

Despite the hereinabove mentioned observation of Lerman and Minna who found no aberrant expression of the RBM5 transcript in lung cancer cell lines, Oh and co-workers could show a reduced expression of RBM5 (i.e., H37) in 9 out of 11 primary nonsmall cell lung cancers tested when compared with neighbouring normal bronchial cells. Furthermore, introduction of II37 cDNA into human breast cancer cells with deletion of 3p22-p21 reduced both anchorage-dependent and -independent cell growth in vitro [Oh Cancer Res (2002) June 1;62(11):3207-13].

Altogether, these results suggest that RBM5 is a putative tumor suppressor gene which may have play a central role in human lung cancer development.

### Example 7

### Degree Type2 Analysis of the Keratin 17 (KRT17) Gene Product of the HSKERELP Contig

Chimeric events involving the HSKERELP contig were analyzed using EST sequences from from tumor origin, specifically EST sequences were derived from head, neck and uterus tumors. Note, that cell-lines are considered as tumors in this case.

A description of chimeric events involving the HSKERELP contig is provided in Table 21, below.

TABLE 21

| | HSKERELP | Chimeric ests info. | Fusion partner | Partner degree |
|---|---|---|---|---|
| 1 | CHIM006891 | #AVG. EST JUMP: −8 | Near | T05092 (1): |
| | t(17, 19) | #EST: BE774393 | PSCD2: pleckstrin homology, | CHIM006891 |
| | | #LIB: UM0009 | Sec7 and coiled/coil domains 2 | |
| | | #mixed normal and tumor | (cytohesin-2) | |
| | | #TISSUES LIST: | | |
| | | uterus | | |
| 2 | CHIM003937 | #AVG. EST JUMP: −4 | tropomyosin-2 | Z19459 (7): |
| | t(17, 9) | #EST: AW630732; | | CHIM006633 |
| | | #LIB: NCI_CGAP_GUI | | CHIM003309 |
| | | #TUMOR | | CHIM004113 |
| | | #TISSUES LIST: | | CHIM003937 |
| | | Genitourinary tract | | CHIM002144 |
| | | | | CHIM000900 |
| | | | | CHIM002466 |
| 3 | CHIM00066 | #AVG. EST JUMP: 2 | DRGI | T11206 (2): |
| | t(17, 22) | #EST: AA584331 | developmentally regulated | CHIM000665 |
| | | #LIB: NCI_CGAP_Lar 1 | GTP binding protein 1 | CHIM002456 |
| | | #TUMOR | | |
| | | #TISSUES LIST: | | |
| | | larynx | | |
| 4 | CHIM00938 | #AVG. EST JUMP: −3 | hypothetical protein | No contig |
| | t(17, 15) | #EST: BI062944 | MGC4562 | |
| | | #LIB: UT0117 | | |
| | | #TUMOR | | |
| | | #TISSUES LIST: | | |
| | | uterus | | |
| 5 | CHIM000951 | #AVG. EST JUMP: −6 | cullin 3 | T08366 (2): |
| | t(7, 2) | #EST: BE717377 | | CHIM003785 |
| | | #LIB: HT0776 | | CHIM000952 |
| | | #TUMOR | | |
| | | #TISSUES LIST: | | |
| | | head & neck hypopharynx | | |
| 6 | CHIM004933 | #AVG. EST JUMP: 15 | — | No contig |
| | | #EST: BE004375 | | |
| | | #LIB: BN0105 | | |
| | | #NORMAL (cell line) | | |
| | | #TISSUES LIST: | | |
| | | marmary gland | | |
| 7 | CHIM004957 | #AVG. EST JUMP: −9 | karyopherin alpha I | Z28442 (2): |
| | 17, 3 | #EST: BG998651 | | CHIM008521 |
| | | #LIB: HT0986 | | CHIM004957 |
| | | #TUMOR | | |
| | | #TISSUES LIST: | | |
| | | head & neck larynx | | |
| 8 | CHIM006318 | #AVG. EST JUMP: 102 | C20orf178 | R11201 (5): |
| | 17, 20 | #EST: BG876291 | similar to dJ553F4.4 (Novel | CHIM007225 |
| | | #LIB: CT0386 | protein similar to | CHIM006790 |
| | | #TUMOR | Drosophila CG8055 protein) | CHIM001227 |
| | | #TISSUES LIST: | (H. sapiens) | CHIM005332 |
| | | colon | | CHIM006318 |

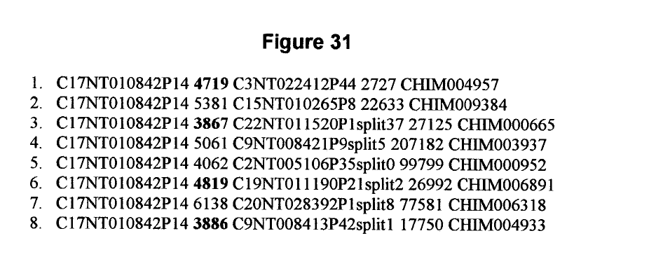

As shown in FIG. 31, chimeric events (i.e., breakoints) 1,6 are distant from each other in 100 bp while chimeric events (i.e., breakoints) 3 and 6 are distant by only 19 bp from each other.

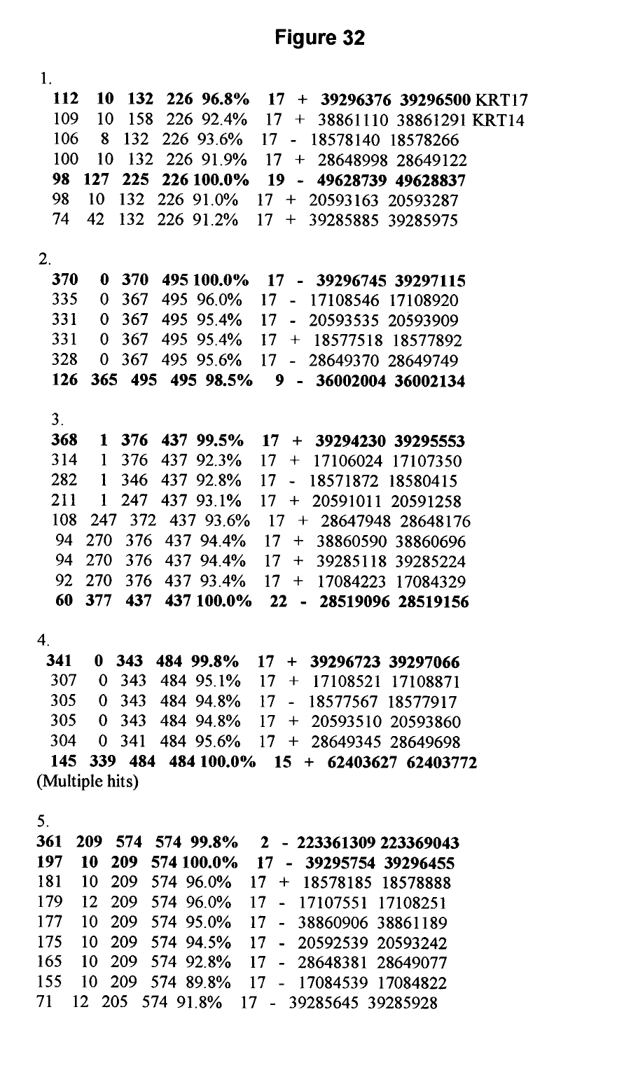

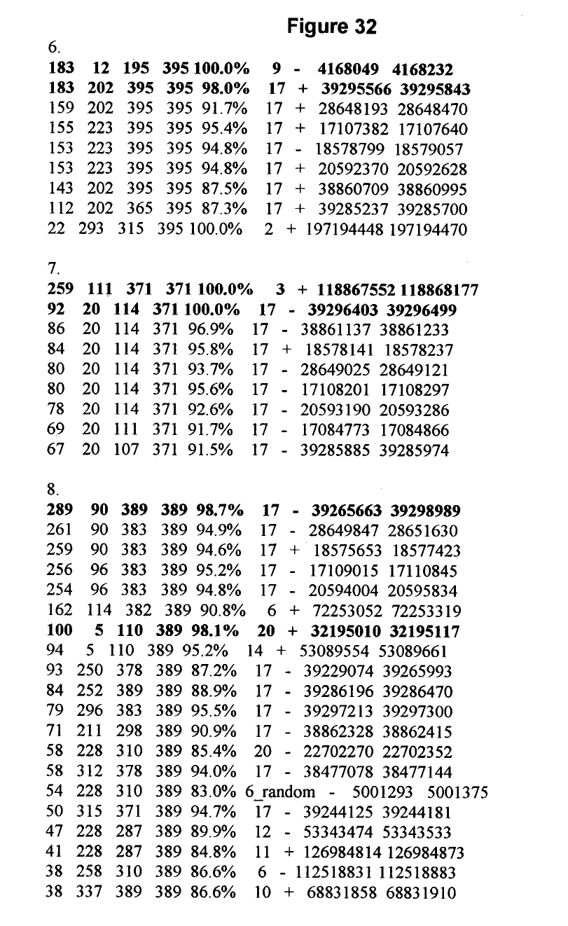

Alignments of the chimeric sequences to the genome is shown in FIG. 32. Noteworthy is that KRT genes are implicated in cancer.

The following describes a linkage between the KRT genes to disease based on swiss-prot databse annotations:

(i) KRT16 and KRT17 are coexpressed only in pathological situations such as metaplasias and carcinomas of the uterine cervix and in psoriasis vulgaris.

(ii) Defects in KRT17 are a cause of type ii pachyonychia congenita (pc-2) also known as jackson-lawler (J and L) syndrome. pc-2 is characterized by onchyogryposis, limited plantar hyperkeratosis, multiple epidermal cysts, abnormal eyebrow and body hair and by the presence of natal teeth.

(iii) Defects in KRT17 are a cause of steatocystoma multiplex (sm), a disease characterized by round or oval cystic tumors widely distributed on the back, anterior trunk, arms, scrotum, and thighs.

### Example 8

### A Polynucleotide Database Storing Sequences Corresponding to the Fusion Transcripts Identified by the Present

Fusion transcript sequences identified according to the teachings of the present invention and their related polynucleotide sequences (i.e., parental sequences building up the fusion transcript) are provided in the CD-ROMs enclosed herewith. File content: “chimeric_contigs_information”, contains summarized data pertaining to each fusion transcript sequence, file “translocated_transcripts126.txt.gz” contains the actual polynucleotide sequences.

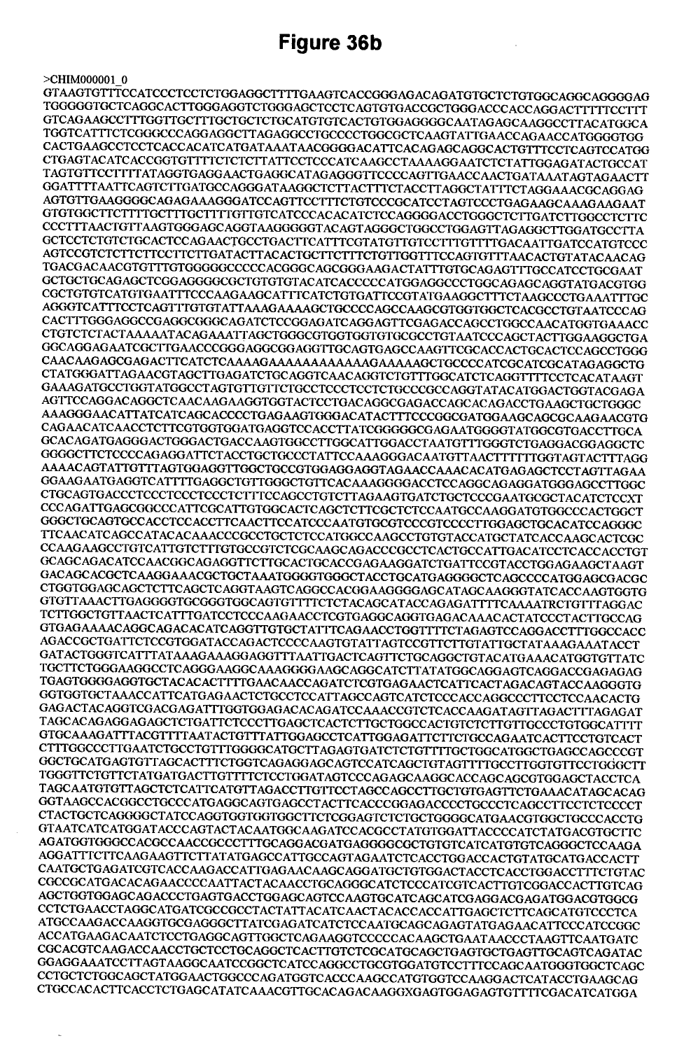

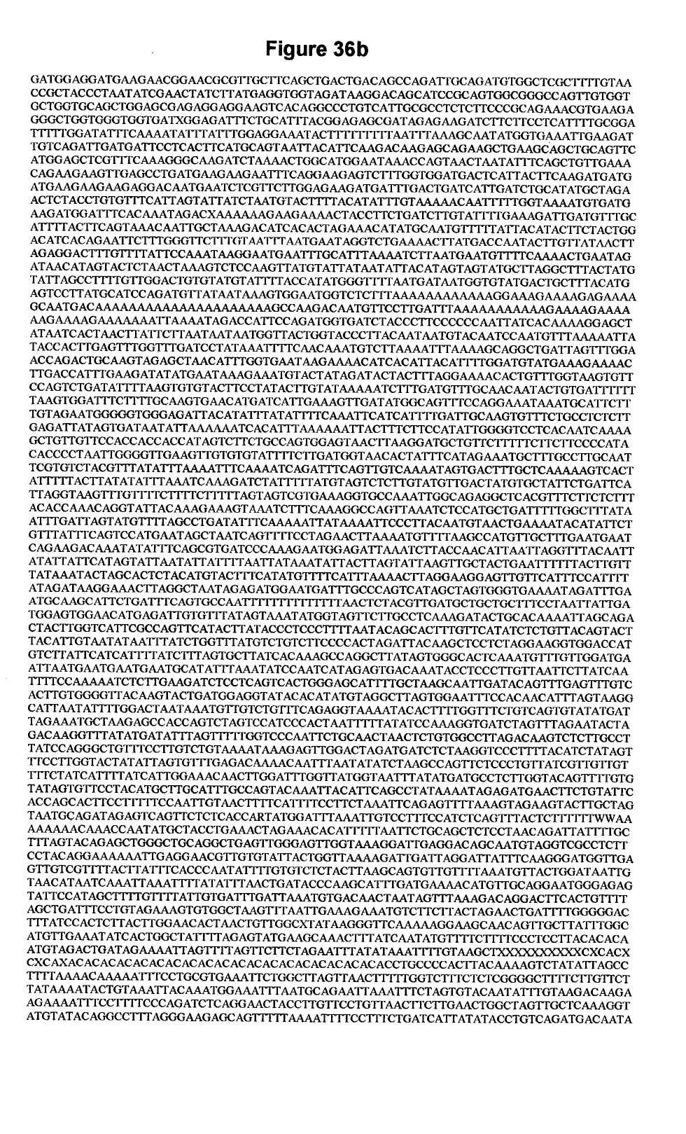

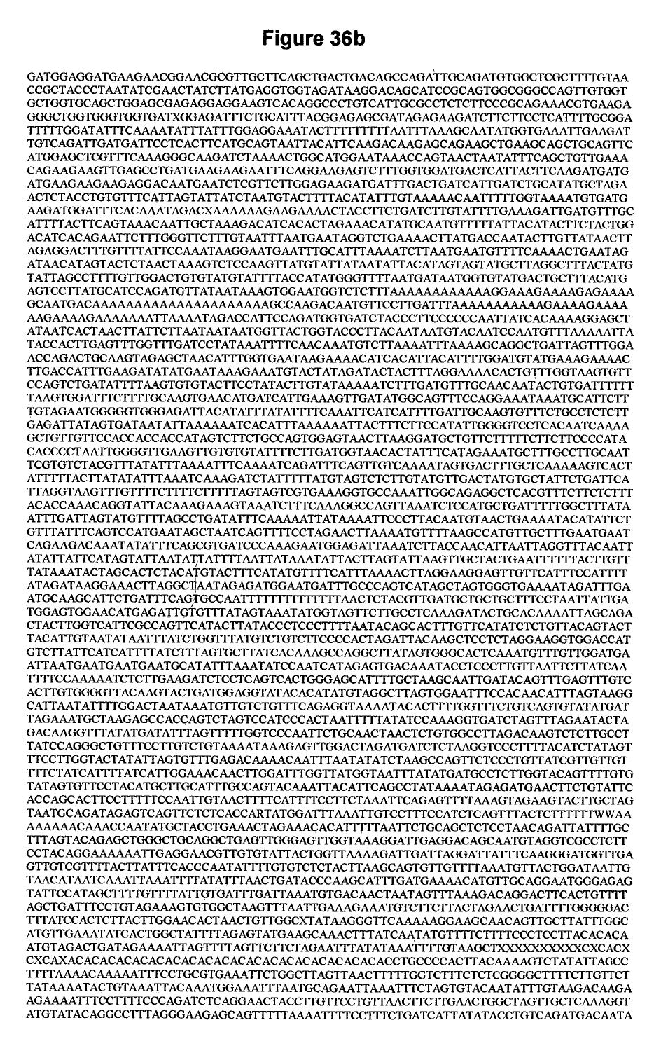

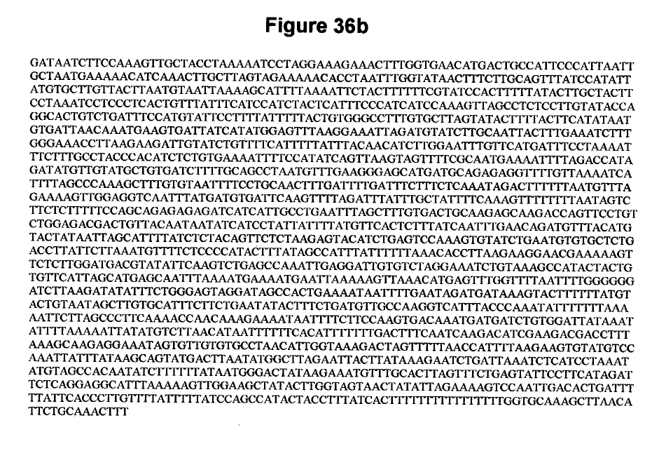

FIG. 36a exemplifies the format of the file “chimeric-contigs-information” provided in the CD-ROM enclosed herewith. FIG. 36a covers a list of fission transcripts. Each transcript is identified by; left and right original contig names, showing GenBank accession numbers and annotation of parental polynucleotides, which flank the transition point; numbers of splice variants (chimeric transcripts); average EST jump; numbers of chimeric ESTs supporting the fusion event; library distribution and tissue origin, the latter two are provided in GenBank annotation.

FIG. 36b shows a sequence example of a Fasta file showing the actual sequences of the chimeric transcripts. Each transcript is arbitrarily designated by the chimeric contig name (e.g., CHIM000001), followed by the chimeric transcript numbers (e.g., 0). The file contains 11302 chimeric contigs, representing 22,607 chimeric transcripts.

The putative fusion transcripts identified by the present invention and disclosed in the enclosed CD-ROM can be used to detect and/or treat a variety of diseases, disorders or conditions, examples of which are listed hereinunder. For example, fusion transcripts or sequence information derived therefrom can be used to construct microarray kits (described in details in the preferred embodiments section) dedicated to diagnosing predisposition to specific diseases, disorders or conditions.

Examples of diseases and proteins which participate in progression of diseases, which diseases can be diagnosed/treated using information derived from fusion transcripts such as those uncovered by the present invention are provided in U.S. Pat. Nos. 6,350,885, 6,245,811, 6,242,419, 6,180,611 and 5,912,233 and in http://www.geneontology.org/.

### Example 9

### Detection of PAX3-FKHR Fusion Transcript of Alveolar Rhabdomyosarcoma by Computational Analysis

Background

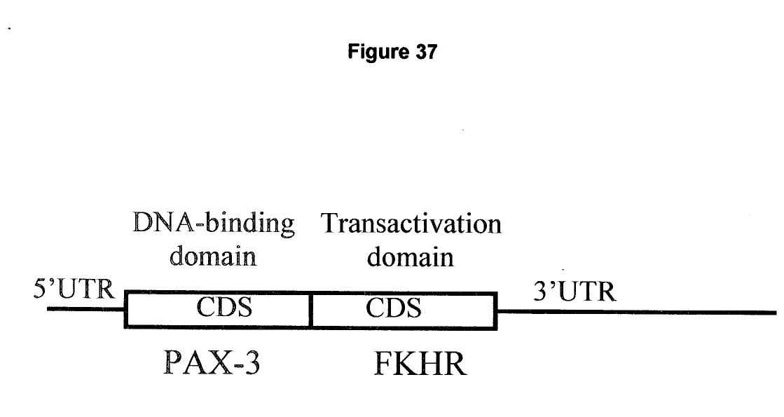

Alveolar rhabdomyosarcoma (ARMS) is a pediatric soft tissue tumor that is associated with either a t(2;13)(q35;q14) or variant t(1;13)(p36;q14) translocation. These translocations fuse either PAX3 or PAX7 with FKHR to generate chimeric genes that express PAX3;FKHR or PAX7;FKHR fusion products, respectively. The fusion proteins consist of the N-terminal DNA-binding domains of PAX3 or PAX7 fused to the C-terminal transcriptional activation domain of FKHR. Transient transfection experiments indicate that PAX3;FKHR functions as a transcription factor. Moreover, PAX3;FKHR exhibits increased transcriptional potency relative to PAX3 due to swapping of PAX3 and FKHR C-terminal transactivation domains (see FIG. 37). Cell culture experiments also have demonstrated that PAX3;FKHR can induce phenotypic changes, including cellular transformation and inhibition of myogenic differentiation [Barr F G (2001) Oncogene 20(40).5736-46].

The ARMS associated PAX3-FKHR translocation was used to assess Leads™ assembly program modified to uncover genetic rearrangements.

Results

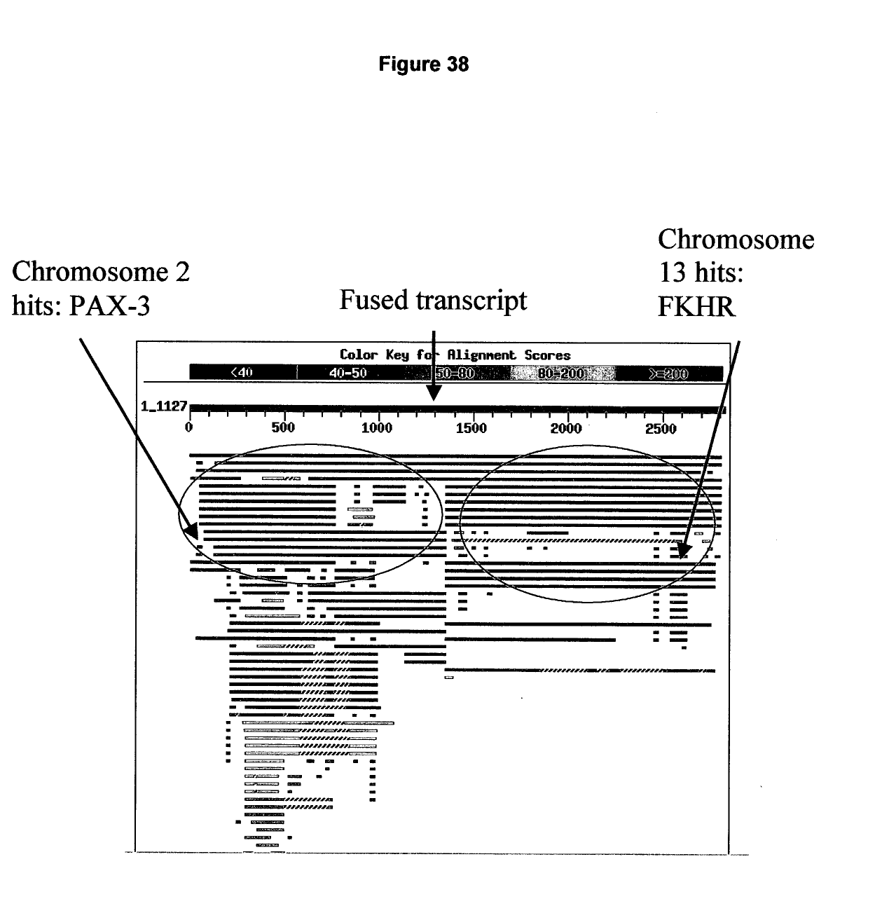

As shown is FIG. 38, PAX3-1 FKHR t(2;13)(q35;q14) translocation was recovered by the modified Leads™ software of the present invention, Specifically, 1 EST from unknown source (SEQ ID NO: 1) and 2 mRNA sequences (SEQ ID NOs: 2 and 3), one of which derived firm alveolar rhabdomyosarcoma exhibited the t(2; 13)(q35;q14) chromosomal translocation.

### Example 10

### Gene Fusion Involving Calmodulin-I and the Matrix Gla Protein

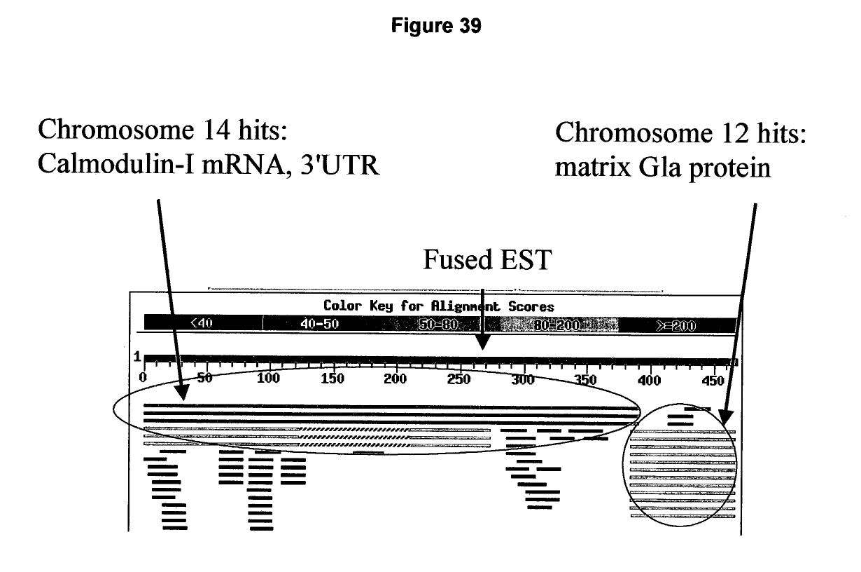

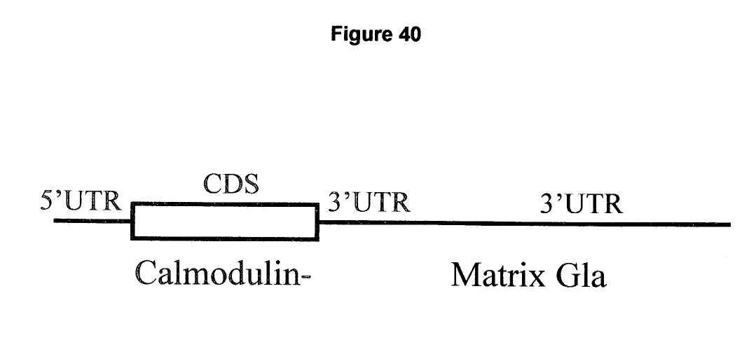

Leads™ assembly program modified to uncover novel genetic rearrangements was used to discover a novel chromosomal translocation corresponding to t(14;12)(q24-31;p13.1-12.3), shown in FIG. 39. This translocation was supported by 7 ESTs (SEQ ID NOs: 4-10) derived from two different breast-specific mini libraries (The Ludwig institute of cancer research, SP, Brazil). The juxtaposition resulted in chimeric transcript comprising the 5′ untranslated region (UTR) and complete coding sequence of calmodulin-I (GenBank Accession No: HSCALMI2) and the 3′UTR of Matrix Gla protein (GenBank Accession No: BC005272), schematically illustrated in FIG. 40.

Aside from the high abundance of ESTs supporting the translocation event, other observations further substantiated it:

1. Sequence analysis of the genomic region 14q30 exhibit multiple TCR sequences, including recombination signal sequences, which are often involved in genetic rearrangements (see the Background section).

2. 12q13 translocations have been reported to be associated with a variety of benign and malignant tumors such as chronic lymphocytic leukemia [Santulli et al (2000) Cancer Genet. Cytogenet. 119(1):70-3] and acute myeloid leukemia [Wong et al. (1999) Cancer Genet. Cytogenet. 114(2):159-61].

3. 12q13 translocations occur more frequently in acute myeloid leukemia with a prior history of mutagenic exposure or karyotypic indicators of secondary leukemia [Wong et al. (1999) Cancer Genet. Cytogenet. 114(2):159-61].

4. Matrix Gla protein has been shown to be involved in oncogenesis and overexpression of which was found in malignant breast carcinoma cells [Clen L. et al. (1990) 5:1391-5] as well as in primary renal-cell carcinomas, prostate carcinomas and testicular germ-cell tumors [Levedakou E N Int J Cancer (1992) 52(4)534-7].

5. Mutations in Matrix Gla proteins were shown to be involved in Keutel syndrome an autosomal recessive disorder characterized by abnormal cartilage calcification, peripheral pulmonary stenosis and midfacial hypoplasia genetically linked to chromosome 12p12.3-13.1 [Munroe P B (1999) Nat. Genet. 21:142-4].

6. Calmodulin a ubiquitous, calcium-binding protein has been shown to regulate cellular proliferation and to be deregulated in malignancies (Hait W N et at. (1986) J. Clin. Oncol. 4:994-1012].

### Example 11

### FE65-Like 2 and AD037 Gene Fusion

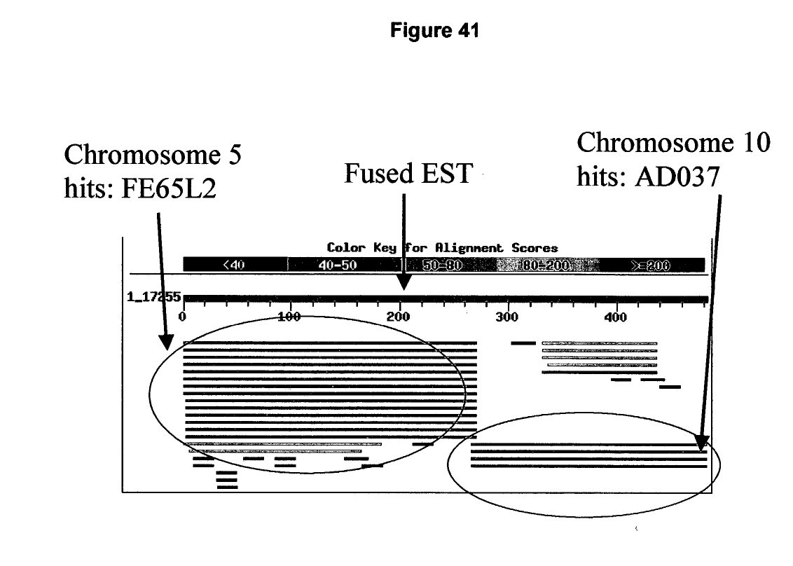

Leads™ assembly program modified to uncover novel genetic rearrangements was used to discover a novel chromosomal translocation corresponding to t(5;10)(q23-q31;incomplete genomic mapping), shown in FIG. 41. This translocation was supported by 3 ESTs (GenBank Accession NOs: AA976815, AI968150, AW206671, SEQ ID NOs: 11-13, respectively) derived from pooled germ cell tumors.

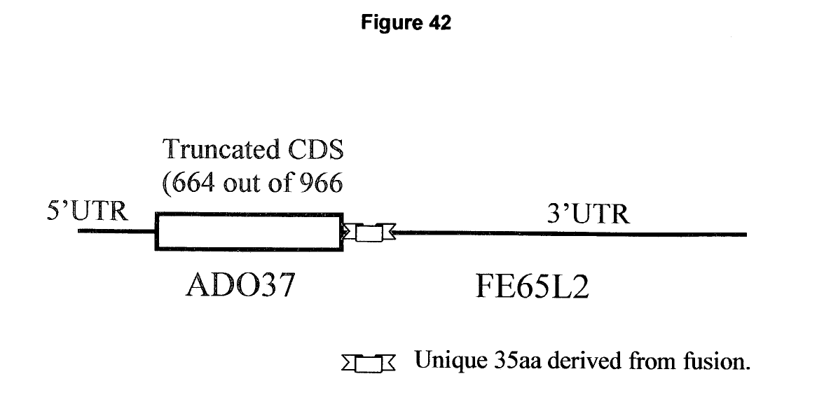

The juxtaposition resulted in chimeric transcript comprising the 5′ UTR and a truncated coding sequence (664/966 bp) of AD037 (GenBank Accession No: gi14042936) followed by a unique sequence encoding 35 amino acids, resulting from the fusion event, as well as the 3′UTR of FE65-like 2 (FE65L2, GenBank Accession No: gi14727694), schematically illustrated in FIG. 42 (SEQ ID NOs: 23-26).

Several findings support the involvement of this translocation event in human pathologies including the observation that AD037 contains a Ras association domain of AF-6 (RalGDS/AF6), known to mediate Ras effect on proliferation and to further be involved in chromosomal rearrangements such as t(6.11) associated with acute lymphoblastic leukemias [Boettner B et al (2000) Proc. Natl. Acad. Sci 97:9064-9069]. In addition, FE65L2 protein has been reported to bind the intracellular domain of the Alzheimer's disease amyloid precursor [Dulio A. et al. (0988) Biochem. J. 330:513-9].

### Example 12

Interleukin-1 Receptor Antagonist and ADP-Ribosyltransferase-like-I Gene Fusion

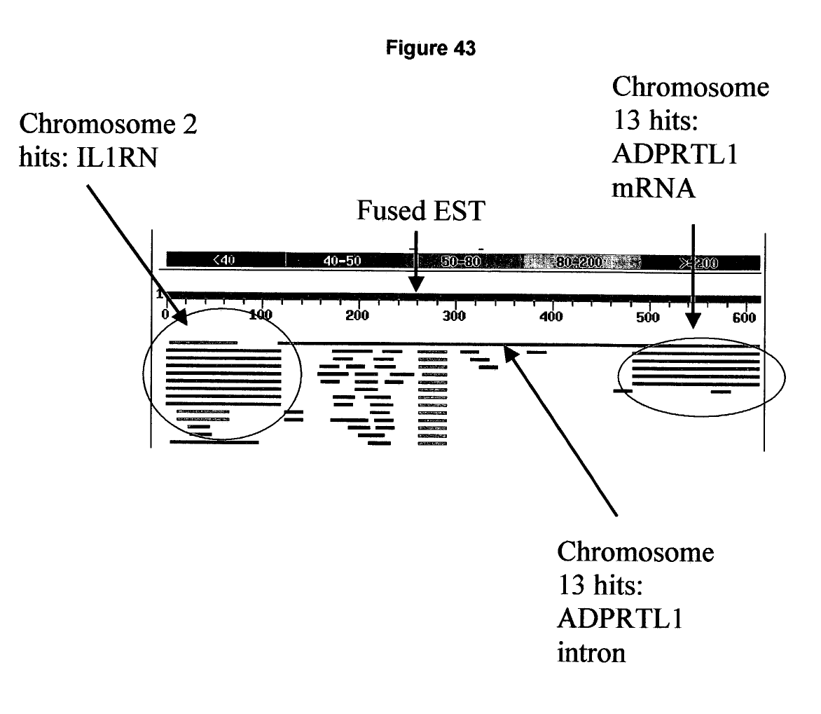

Leads™ assembly program modified to uncover novel genetic rearrangements was used to discover a novel chromosomal translocation corresponding to t(2d;13)(q14;q 11), shown in FIG. 43. This translocation was supported by 2 ESTs (AA527443 and AW972255, SEQ ID NOs: 14-15, respectively) derived from colon tumors.

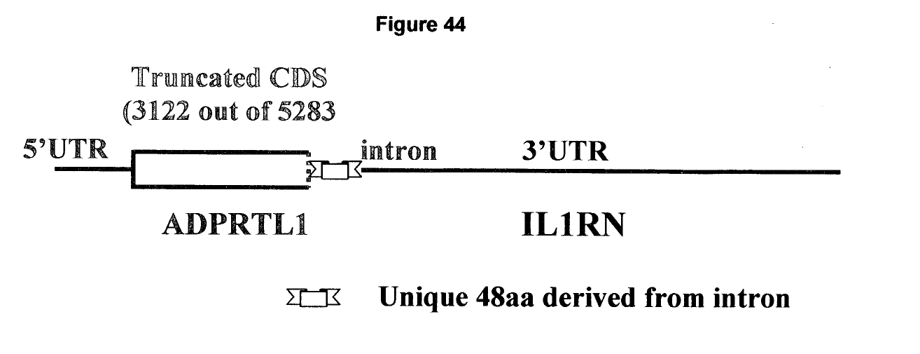

The juxtaposition resulted in a chimeric transcript comprising the 5′ UTR and truncated coding sequence (3122/5283 bp) of ADP-ribosyltransferase-like-I (ADPRTL1) (GenBank Accession No. 13649591) followed by a unique sequence encoding 48 amino acids, derived from intron retention resulting from the fusion event (GenBank Accession No: gi10045472), as well as the 3′UTR of Interleukin 1 receptor antagonist (IL1RN, GenBank Accession No: gi15321361), schematically illustrated in FIG. 44 (SEQ ID NO: 27).

The putative involvement of this fusion in human pathologies is supported by the observation that maintenance of a balance between IL1 and its antagonist IL1Ra, an IL1RN isoform, is important in preventing the development or progression of inflammatory disease in certain organs. Both the secreted and intracellular isoforms of IL1Ra contribute to maintenance of this balance. An allelic polymorphism in intron 2 of the IL1Ra gene (IL1RN*2) predisposes to the development or severity of a variety of human diseases largely of epithelial cell origin [Arend W P, Guthridge C J (2000) Ann. Rheum. Dis. 59 (1):160-4]. Indeed, IL1RN is found to be in strong association with many diseases such as multiple sclerosis, osteoporposis and a variety of infectious, immune and traumatic conditions such as systemic lupus erythematosus, ulcerative colitis, alopecia arcate vulvar vestibulitis, and possibly osteoporosis and coronary artery disease [Witkin et al. (2002) Clin Infect Dis. 34(2):2049]. Furthermore, proinflammatory genotypes of the IL1 loci increase the risk of gastric cancer.

### Example 13

### NADPH Thyroid Oxidase 2 and Stromal Cell-Derived Factor 2-Like Gene Fusion

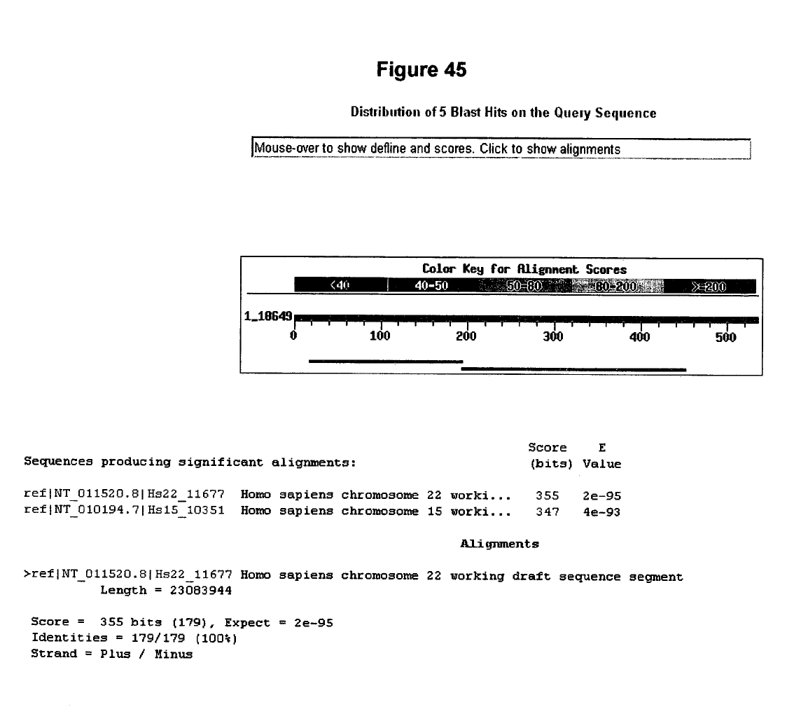

Leads™ assembly program modified to uncover novel genetic rearrangements was used to discover a novel chromosomal translocation corresponding to (15;22)(15q15.3-q21)(22q11.21) shown in FIG. 45. This translocation was supported by 2 ESTs (SEQ ID Nos. 16 and 17, GenBank Accession Nos.: AI700319 and AA533569) derived from two different libraries generated from colon and ovarian tissues.

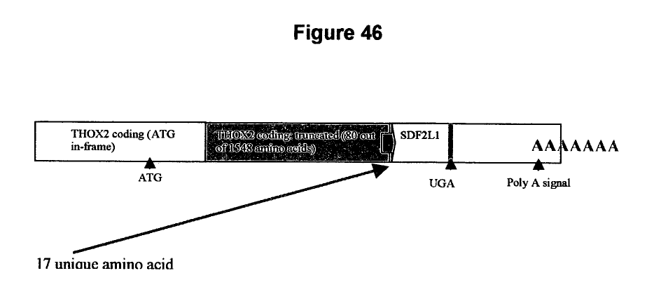

The juxtaposition resulted in a chimeric transcript comprising the 5′ UTR and truncated coding sequence (240/4644 bp) of NADPH thyroid oxidase 2 (THOX-2, GenBank Accession. No: gi8163927) followed by a unique 17 unique amino acids, derived from an alternative open-reading frame of the stromal cell-derived factor 2-like-1 sequence (SDF2L1, GenBank Accession No: gi/1593R03) as well as the coding sequence and 3′UTR of the gene (illustrated in FIG. 46).

The putative involvement of this fusion in human pathologies., especially those associated with malfunctioning of the thyroid gland is supported by the following observations; THOX2, a Calcium-dependent flavoprotein component of the thyroid NADPH oxidase, which includes a ferric reductase like transmembrane component and to be mapped to chromosome 15 has been shown to bee up-regulated by cAMP in the rat thyroid cell-line FRTL-5 and down-regulated in rat thyroids treated with the antithyroid drug methimazole [Dupuy C. et al. (2000 Biochem Biophys Res Commun 277(2)287-92]. Furthermore the SDF2L1 gene (chromosome 22), which includes a mannosyltransferase, IP3R and RyR (MIR) domain implicated in ligand transferase function, has been shown to be an endoplasmic reticulum stress-inducible gene, which may be implicated in various disorders [Fukuda S et al. (2001) Biochem Biophys Res Commun 280(1):407-14].

### Example 14

### Endonuclease and Sterile Alpha and HEAT/Armadillo Motif Protein Gene Fusion

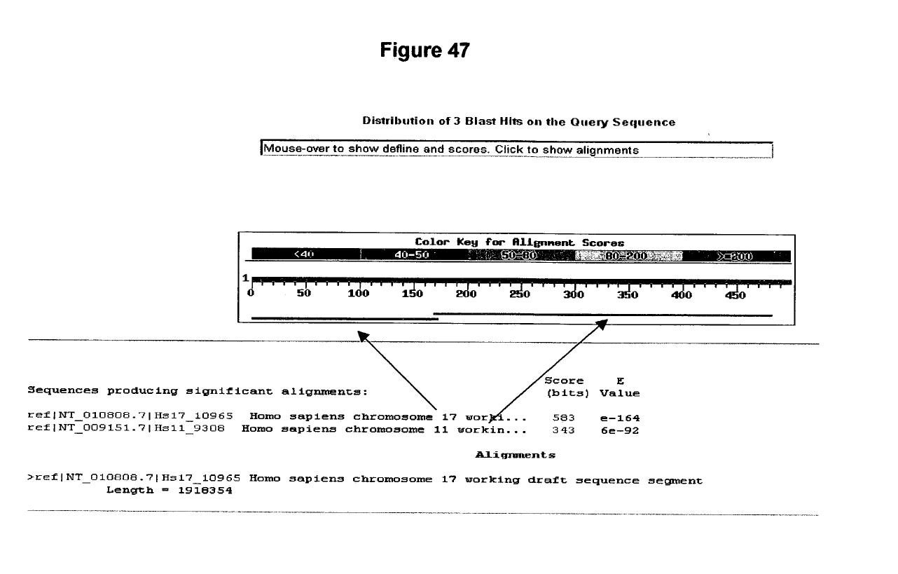

Leads™ assembly program modified to uncover novel genetic rearrangements was used to discover a novel chromosomal translocation corresponding to t(11;17) 11q23.2 (complete chromosomal mapping of the indicated translocation is currently not available), shown in FIG. 47. This translocation was supported by 5 ESTs (SEQ ID Nos. 18-22; GenBank Accession Nos.: AW972641 AW972443 AA508759 AA508773 and AA528039) derived from colon tumors.

The juxtaposition resulted in a chimeric transcript comprising exon 5 of the sterile alpha and HEAT/Armadillo motif (SARM) protein, ortholog of Drosophila GenBank accession No: gi/14774013 (Chromosome 17) and exon 7 of the LOC120398, a region containing RNA binding motif protein 7, having an endonuclease activity (Chromosome 11).

The following models where used to explain the fusion event:

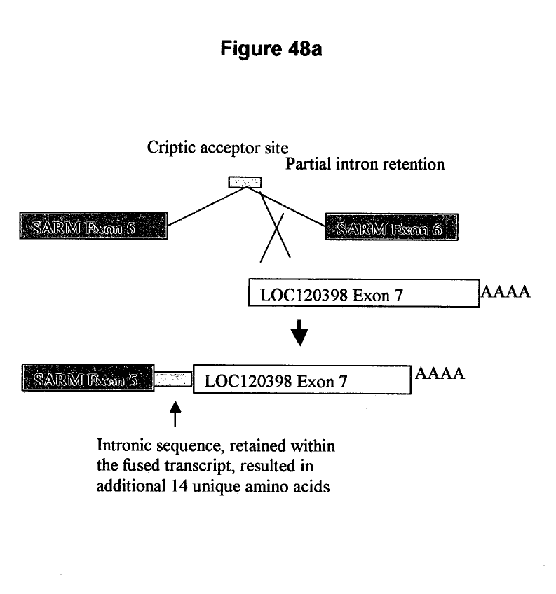

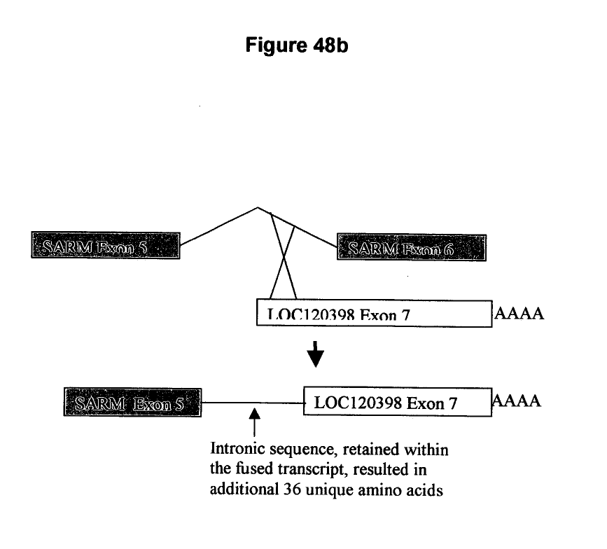

As shown in FIG. 48a following the translocation event, an alternative criptic acceptor site between exons 5 and 6 of the SARM gene became active, causing partial intron retention. This resulted in SARM truncated protein, containing 482 out of 690 amino acids, and an additional 14 unique amino acids derived from a partial intron sequence. FIG. 48b illustrates an alternative fusion model, in which following the fusion event, the original acceptor site between exons 5 and 6 of the SARM gene was deleted. This resulted in SARM truncated protein, containing 482 out of 690 amino acids and additional 36 amino acids.

### Example 15

### A database of Chimeric Events According to a Degree Type II Analysis

The file content “degree_{—}2_analysis” on the enclosed CAROM contains summarized data pertaining to degree type 2 analysis of several fusion transcript sequences in the file “translocated_transcripts126.txt.gz”.

Note that each paragraph within the “degree-2_analysis” file provides information pertaining to a different contig.

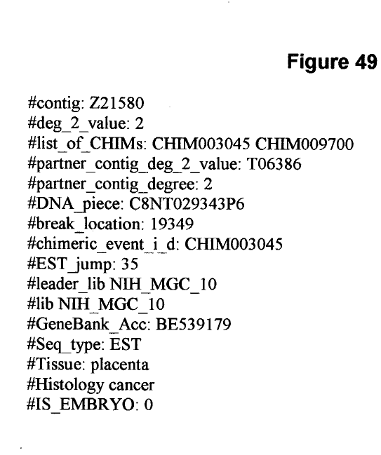

FIG. 49 illustrates an example for a degree_{—}2_analysis” file.

Data format—

#contig: <name of contig>

a contig is a gene or a part of a gene created by LEADS™ (Comnpugen, Tel-Aviv, Israel).

This contig is the pivot contig in the case.

#deg_{—}2_value: <number>

Degree type 2 value. This value is the number of the chimeric events of the pivot contig.

#list_of_CHIMs: <list>

A list of the chimeric event serial numbers which are involved with the pivot contig.

#partner-contig_degree: <number>

Degree type 2 value of the partner contigs. This value is the number of the chimeric event of the partner contig.

#DNA-piece: <name>

the name of the DNA sequence that the contig aligns to. The name of the sequence was taken from the GANG algorithm output

#break_location: <number>

Coordinates on the DNA sequence. The coordinates specify the left end of the alignment between the chimeric sequence and the DNA sequence.

#chimeric_event_i_d: <name>

The serial name of the chimeric event.

#EST_jump: <number>

The value of the EST jump of the chimeric sequence in the chimeric event.

#leader_lib <cDNA library name>

The name of the cDNA library. This library represents the “parent” library of the chimeric DNA sequence. It means that the cDNA library of the chimeric sequence originate from the same clone, tissue sample or pool Of tissues/clones of the parent library.

#lib <cDNA library name>

A name of a cDNA library. The chimeric sequence originates from this library.

#GeneBank_Acc: <name>

The GeneBank accession of the chimeric sequence.

#Seq_type: <EST/mRNA>

The type of the chimeric sequence.

#Tissue: <name>

The tissue used to prepare the cDNA library.

#Histology <histology>

The histology of the tissue used to prepare the cDNA library.

#IS_EMBRYO: <0/2>

This field specifies the embryonic origin of the tissue.

0 denotes that the tissue is not originated from embryo.

2 denotes that the tissue is from an embryonic origin.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by their accession number was specifically and individually indicated to be incorporated herein by reference. In addition, citation or identification of any reference in this application shall not be construed as an admission that such reference is available as prior art to the present invention.

### CD-ROM Content

The following lists the file content of the duplicate CD-ROMs which are enclosed herewith and filed with the application. These files care incorporated herein by reference and thus form a part of the filed application. File information is provided as: File name/bite size/date of creation/operating system/machine format.

Chimeric-contigs-information.txt/11821000/02.27.2002/WINDOWS/IBMPC

Translocated_transcripts 126.txt.gz/245106000/02.27.2002/WINDOWS/IBMP C

Degree_{—}2_analysis.txt/6540000/02.18.2003/WINDOWS/IBMPC

What is claimed is:

1. A method of identifying putative fusion transcripts, the method comprising:
(a) computationally aligning a first database of annotated polynucleotide sequences with a second database of expressed polynucleotide sequences; and
(b) identifying in said second database an expressed polynucleotide sequence complementary to at least two non-contiguous sequences of said first database, said at least two non-contiguous sequences being selected from the group consisting of non-homologous polynucleotide sequences mapped to different chromosomes, polynucleotide sequences mapped to different loci of a single chromosome and polynucleotide sequences mapped to a single locus and not being a part of a splice isoform, said expressed polynucleotide sequence identified being a putative fusion transcript.

2. The method of claim 1, further comprising the step of testing the putative fusion transcript for the presence of at least one sequence element selected from the group consisting of a sequence repeat, a pseudogene sequence, a restriction site, a transposable element, a homologous sequence, a sequence direction, overhang length, a splice consensus site, an intron length, a transcript length, alignment score, a hotspot sequence, a vector sequence, a gap, a sequence conservation and an EST jump.

3. The method of claim 1, wherein said first annotated database includes sequences of a type selected from the group consisting of genomic sequences, expressed sequence tags, contigs, complementary DNA (cDNA) sequences, pre-messenger RNA (mRNA) sequences and mRNA sequences.

4. The method of claim 1, wherein said second database includes sequences of a type selected from the group consisting of expressed sequence tags, contigs, complementary DNA (cDNA) sequences, pre-messenger RNA (mRNA) sequences and mRNA sequences.

5. The method of claim 1, wherein the putative fusion transcripts are selected from the group consisting of translocation products, deletion products, duplication products, paracentric inversions, pericentric inversions, transpositions, ring chromosomes, trans-splicing products and trans-transcription products.

6. A method of identifying transition points in fusion transcripts, the method comprising:
(a) computationally aligning a first database of annotated polynucleotide sequences with a second database of expressed polynucleotide sequences; and
(b) selecting in said second database an expressed polynucleotide sequence complementary to at least two non-contiguous sequences of said first database, said at least two non-contiguous sequences being selected from the group consisting of non-homologous polynucleotide sequences mapped to different chromosomes, polynucleotide sequences mapped to different loci of a single chromosome and polynucleotide sequences mapped to a single locus and not being a part of a splice isoform, and
(c) identifying within the putative fusion transcript at least one nucleic acid sequence region exhibiting transition point between a first contiguous sequence of said at least two non-contiguous sequences and a second contiguous sequence of said at least two non-contiguous sequences, thereby identifying the transition points in the fusion transcripts.

7. The method of claim 6, further comprising the step of testing the putative fusion transcript for the presence of at least one sequence element selected from the group consisting of a sequence repeat, a pseudogene sequence, a restriction site, a transposable element, a homologous sequence, a sequence direction, overhang length, a splice consensus site, an intron length, a transcript length, alignment score, a hotspot sequence, a vector sequence, a gap, a sequence conservation and an EST jump.

8. The method of claim 6, wherein said first annotated database includes sequences of a type selected from the group consisting of genomic sequences, expressed sequence tags, contigs, complementary DNA (cDNA) sequences, pre-messenger RNA (mRNA) sequences and mRNA sequences.

9. The method of claim 6, wherein said second database includes sequences of a type selected from the group consisting of expressed sequence tags, contigs, complementary DNA (cDNA) sequences, pre-messenger RNA (mRNA) sequences and mRNA sequences.

10. The method of claim 6, wherein the putative fusion transcripts are selected from the group consisting of translocation products, deletion products, duplication products, paracentric inversions, pericentric inversions, transpositions, ring chromosomes, trans-splicing products and trans-transcription products.

11. A method of identifying polynucleotide sequences associated with a disorder associated with genetic rearrangements, the method comprising:
(a) computationally aligning a first database of annotated polynucleotide sequences with a second database of expressed polynucleotide sequences derived from tissues characterized by disorders associated with genetic rearrangements;
(b) identifying in said second database an expressed polynucleotide sequence complementary to at least two non-contiguous sequences of said first database, said at least two lion-contiguous sequences being selected from the group consisting of non-homologous polynucleotide sequences mapped to different chromosomes, polynucleotide sequences mapped to different loci of a single chromosome and polynucleotide sequences mapped to a single locus and not being a part of a splice isoform, and
(c) identifying said non-contiguous polynucleotide sequences of said first database to thereby identify the polynucleotide sequences associated with disorders associated with genetic rearrangements.

12. The method of claim 11, further comprising the step of testing the polynucleotide sequences being associated with the disorder associated with genetic rearrangements for pathogenic potential under physiological conditions following step (c).

13. The method of claim 11, further comprising the step of testing the putative fission transcript for the presence of at least one sequence element selected from the group consisting of a sequence repeat, a pseudogene sequence, a restriction site, a transposable element, a homologous sequence, a sequence direction, overhang length, a splice consensus site, an intron length, a transcript length, alignment score, a hotspot sequence, a vector sequence, a gap, a sequence conservation and an EST jump.

14. The method of claim 11, wherein said first annotated database includes sequences of a type selected from the group consisting of genomic sequences, expressed sequence tags, contigs, complementary DNA (cDNA) sequences, pre-messenger RNA (mRNA) sequences and mRNA sequences.

15. The method of claim 11, wherein said second database includes sequences of a type selected from the group consisting of expressed sequence tags, contigs, complementary DNA (cDNA) sequences, pre-messenger RNA (mRNA) sequences and mRNA sequences.

16. A method of identifying polypeptides resulting from putative fusion events, the method comprising:
(a) computationally aligning a first database of annotated polynucleotide sequences with a second database of expressed polynucleotide sequences;
(b) identifying in said second database expressed polynucleotide sequences complementary to at least two non-contiguous sequences of said first database, said at least two non-contiguous sequences being selected from the group consisting of non-homologous polynucleotide sequences mapped to different chromosomes, polynucleotide sequences mapped to different loci of a single chromosome and polynucleotide sequences mapped to a single locus and not being a part of a splice isoform, % said expressed polynucleotide sequences identified being fusion transcripts; and
(c) selecting from said fusion transcripts at least one fusion transcript including an open reading frame spanning at least one of said at least two non-contiguous sequences, thereby identifying the polypeptides resulting from the putative fusion event.

17. The method of claim 16, further comprising the step of testing said fusion transcripts for the presence of at least one sequence element selected from the group consisting of a sequence repeat, a pseudogene sequence, a restriction site, a transposable element, a homologous sequence, a sequence direction, overhang length, a splice consensus site, an intron length, a transcript length, alignment score, a hotspot sequence, a vector sequence, a gap, a sequence conservation and an EST jump.

18. The method of claim 16, wherein said first annotated database includes sequences of a type selected from the group consisting of genomic sequences, expressed sequence tags, contigs, complementary DNA (cDNA) sequences, pre-messenger RNA (mRNA) sequences and mRNA sequences.

19. The method of claim 16, wherein said second database includes sequences of a type selected from the group consisting of expressed sequence tags, contigs, complementary DNA (cDNA) sequences, pre-messenger RNA (mRNA) sequences and mRNA sequences.

20. The method of claim 16, wherein said fusion transcripts are selected from the group consisting of translocation products, deletion products, duplication products, paracentric inversions, pericentric inversions, transpositions, ring chromosomes, trans-splicing products and trans-transcription products.

21. A kit useful for detecting genetic rearrangements, the kit comprising at least one oligonucleotide being designed and configured to be specifically hybridizable with at least one fusion transcript of the fusion transcripts set forth in the file “translocated_transcripts126.txt.gz”.

22. The kit of claim 21, wherein said at least one oligonucleotides is designed and configured to be specifically hybridizable with at least one transition point in said at least one fusion transcript of the fusion transcripts set forth in the file “translocated_transcripts126.txt.g/z”.

23. The kit of claim 21, wherein said at least one oligonucleotide is labeled.

24. The kit of claim 21, wherein said at least one oligonucleotide is attached to a solid substrate.

25. The kit of claim 24, wherein said solid substrate is configured as a microarray and whereas said at least one oligonucleotide includes a plurality of oligonucleotides each being capable, or hybridizing with a specific fusion transcript of the fusion transcript set forth in the file “translocated_transcripts126.txt.gz” and each being attached to said microarray in a regio-specific manner.

26. The kit of claim 21, wherein said at least one oligonucleotide is designed and configured for DNA staining.

27. The kit of claim 21, wherein said at least one oligonucleotide is designed and configured for RNA staining.

28. A computer readable storage medium comprising data stored in a retrievable manner, said data including sequence information of at least a portion of the fusion transcripts set forth in file “translocated_transcripts126.txt.gz”.

29. The computer readable storage medium of claim 28, wherein said data further includes additional information specific to each transcript of said at least a portion of the fusion transcripts.

30. The computer readable storage medium of claim 29, wherein said additional information includes at least one item selected from the group consisting of:
(i) genes functionally or structurally related to each transcript of said at least a portion of the fusion transcripts;
(ii) a sequence length of each transcript of said at least a portion of the fusion transcripts;
(iii) open reading frames and/or regulatory sequences associated with each transcript of said at least a portion of the fusion transcripts;
(iv) transition point sequence between each transcript of said at least a portion of the fusion transcripts;
(v) pathological abundance;
(vi) chromosomal mapping of each transcript of said at least a portion of the fusion transcripts;
(vii) causative genetic event selected from the group consisting of a deletion and translocation, an insertion, erroneous splicing and a trans-splicing.
(viii) EST-jump value;
(ix) hotspot sequences; and
(x) fusion event abundance.

31. The computer readable storage medium of claim 29, wherein said additional information is set forth in the file “chimeric_contigs_information”.

32. The computer readable storage medium of claim 28, wherein said database further includes information pertaining to generation of said data and potential uses of said data.

33. A system for generating a database of fusion transcripts, the system comprising a processing unit, said processing unit executing a software application configured for:
(a) computationally aligning a first database of annotated polynucleotide sequences with a second database of expressed polynucleotide sequences;
(b) identifying in said second database an expressed polynucleotide sequence complementary to at least two non-contiguous sequences of said first database, said at least two non-contiguous sequences being selected from the group consisting of non-homologous polynucleotide sequences mapped to different chromosomes, polynucleotide sequences mapped to different loci of a single chromosome and polynucleotide sequences mapped to a single locus and not being a part of a splice isoform; and
(c) storing the fusion transcripts as retrievable data.

34. The system of claim 33, wherein said software application is further configured for annotating the fusion transcripts stored and whereas said annotation is effected according to data derived from sequences or other databases.

35. The system of claim 33, wherein said software application is further configured for testing the putative fission transcripts for the presence of at least one sequence element selected from the group consisting of a sequence repeat, a pseudogene sequence, a restriction site, a transposable element, a homologous sequence, a sequence direction, overhang length, a splice consensus site, an intron length, a transcript length, alignment score, a hotspot sequence, a vector sequence, a gap, a sequence conservation and an EST jump.

36. The system of claim 33, wherein said first annotated database includes sequences of a type selected from the group consisting of genomic sequences, expressed sequence tags, contigs, complementary DNA (cDNA) sequences, pre-messenger RNA (mRNA) sequences and mRNA sequences.

37. The system of claim 33, wherein said second database includes sequences of a type selected from the group consisting of expressed sequence tags, contigs, complementary DNA (cDNA) sequences, pre-messenger RNA (mRNA) sequences and mRNA sequences.

38. The system of claim 33, wherein the putative fusion transcripts are selected from the group consisting of translocation products, deletion products, duplication products, paracentric inversions, pericentric inversions, transpositions, ring chromosomes, trans-splicing products and trans-transcription products.

39. A system for generating a database of nucleic acid sequences of transition points in fusion transcripts, the system comprising a processing unit, said processing unit executing a software application configured for:
(a) computationally aligning a first database of annotated polynucleotide sequences with a second database of expressed polynucleotide sequences;
(b) selecting in said second database an expressed polynucleotide sequence complementary to at least two non-contiguous sequences of said first database, said at least two non-contiguous sequences being selected from the group consisting of non-homologous polynucleotide sequences mapped to different chromosomes, polynucleotide sequences mapped to different loci of a single chromosome and polynucleotide sequences mapped to a single locus and not being a part of a splice isoform,
(c) identifying within the putative fusion transcript at least one nucleic acid sequence region exhibiting transition point between a first contiguous sequence of said at least two non-contiguous sequences and a second contiguous sequence of said at least two non-contiguous sequences; and
(d) storing the nucleic acid sequences of transition points in fusion transcripts as retrievable data.

40. The system of claim 39, wherein said software application is further configured for annotating the nucleic acid sequences of transition points in fusion transcripts stored and whereas said annotation is effected according to data derived from sequences or other databases.

41. The system of claim 39, wherein said software application is further configured for testing the transition points in fusion transcripts for the presence of at least one sequence element selected from the group consisting of a sequence repeat, a pseudogene sequence, a restriction site, upstream overhang length, downstream overhang length, and a splice consensus site.

42. The system of claim 39, wherein said first annotated database includes sequences of a type selected from the group consisting of genomic sequences, expressed sequence tags, contigs, complementary DNA (cDNA) sequences, pre-messenger RNA (mRNA) sequences and mRNA sequences.

43. The system of claim 39, wherein said second database includes sequences of a type selected from the group consisting of expressed sequence tags, contigs, complementary DNA (cDNA) sequences, pre-messenger RNA (mRNA) sequences and mRNA sequences.

44. The system of claim 39, wherein the fusion transcripts are selected from the group consisting of translocation products, deletion products, trans-splicing products and trans-transcription products.

45. A system for generating a database of polypeptide encoding nucleic acid sequences resulting from putative fusion events, the system comprising a processing unit, said processing unit executing a software application configured for:
(a) computationally aligning a first database of annotated polynucleotide sequences with a second database of expressed polynucleotide sequences;
(b) identifying in said second database expressed polynucleotide sequences complementary to at least two non-contiguous sequences of said first database, said at least two non-contiguous sequences being selected from the group consisting of non-homologous polynucleotide sequences mapped to different chromosomes, polynucleotide sequences mapped to different loci of a single chromosome and polynucleotide sequences mapped to a single locus and not being a part of a splice isoform, said expressed polynucleotide sequences identified being fusion transcripts; and
(c) identifying from said fusion transcripts the polypeptide encoding nucleic acid sequences including an open reading frame spanning at least one of said at least two non-contiguous sequences; and
(d) storing the polypeptide encoding nucleic acid sequences resulting from the putative fusion as retrievable data.

46. The system of claim 45, wherein said software application is further configured for annotating the polypeptide encoding nucleic acid sequences and whereas said annotation is effected according to data derived from sequences or other databases.

47. The system of claim 45, wherein said first annotated database includes sequences of a type selected from the group consisting of genomic sequences, expressed sequence tags, contigs, complementary DNA (cDNA) sequences, pre-messenger RNA (mRNA) sequences and mRNA sequences.

48. The system of claim 45, wherein said second database includes sequences of a type selected from the group consisting of expressed sequence tags, contigs, complementary DNA (cDNA) sequences, pre-messenger RNA (mRNA) sequences and mRNA sequences.

49. The system of claim 45, wherein said fusion transcripts are selected from the group consisting of translocation products, deletion products, trans-splicing products and tans-transcription products.

50. A method of detecting a nucleic acid sequence chimerism indicative of predisposition for disorders associated with genetic rearrangements in a subject, the method comprising:
(a) identifying a fusion transcript indicative of the nucleic acid sequence chimerism;
(b) generating at least one oligonucleotide being complementary to said fusion transcript;
(c) contacting a biological sample obtained from the subject with said at least one oligonucleotide; and
(d) detecting a level of binding between said at least one oligonucleotide and said fusion transcript to thereby detect the nucleic acid sequence chimerism indicative of the predisposition for disorders associated with genetic rearrangements in the subject.

51. The method of claim 50, wherein said at least one oligonucleotide being complementary to said fusion transcript is complementary to a transition point within said fusion transcript.

52. The method of claim 50, wherein the step of identifying said fusion transcript indicative of the nucleic acid sequence chimerism is effected by:
(i) computationally aligning a first database of annotated polynucleotide sequences with a second database of expressed polynucleotide sequences; and
(ii) identifying in said second database expressed polynucleotide sequences complementary to at least two non-contiguous sequences of said first database, said at least two non-contiguous sequences being selected from the group consisting of non-homologous polynucleotide sequences mapped to different chromosomes, polynucleotide sequences mapped to different loci of a single chromosome and polynucleotide sequences mapped to a single locus and not being a part of a splice isoform said expressed polynucleotide sequences identified being fusion transcripts.

53. The method of claim 52, further comprising the step of testing said fusion transcript for the presence of at least one sequence element selected from the group consisting of a sequence repeat, a pseudogene sequence, a restriction site, a transposable element, a homologous sequence, a sequence direction, overhang length, a splice consensus site, an intron length, a transcript length, alignment score, a hotspot sequence, a vector sequence, a gap, a sequence conservation and an EST jump.

54. The method of claim 52, wherein said first annotated database includes sequences of a type selected from the group consisting of genomic sequences, expressed sequence tags, contigs, complementary DNA (cDNA) sequences, pre-messenger RNA (mRNA) sequences and mRNA sequences.

55. The method of claim 52, wherein said second database includes sequences of a type selected from the group consisting of expressed sequence tags, contigs, complementary DNA (cDNA) sequences, pre-messenger RNA (mRNA) sequences and mRNA sequences.

56. The method of claim 52, wherein said fusion transcript is selected from the group consisting of translocation products, deletion products, duplication products, paracentric inversions, pericentric inversions, transpositions, ring chromosomes, trans-splicing products and trans-transcription products.

57. The method of claim 50, wherein said at least one oligonucleotide is attached to a solid substrate.

58. The method of claim 57, wherein said solid substrate is configured as a microarray and whereas said at least one oligonucleotide includes a plurality of oligonucleotides each attached to said microarray in a regio-specific manner.

59. The method of claim 50, wherein said at least one oligonucleotide is labeled and whereas step (d) is effected by quantifying said label.

60. A method of identifying putative mutagenic agents, the method comprising:
(a) exposing a cell to a plurality of mutagens; and
(b) identifying a mutagen of said plurality of mutagens which induces expression of a fusion transcript in said cell as a result of exposure of said cell thereto, thereby identifying the putative mutagenic agent.

61. The method of claim 60, wherein said identifying is effected on RNA target molecules of said plurality of cells.
